(19) United States
(12) Patent Application Publication
(10) Pub. No.: US 2025/0354218 A1
(43) Pub. Date: Nov. 20, 2025

(54) METHODS FOR DETECTING ACUTE MYELOID LEUKEMIA

(71) Applicant: Cornell University, Ithaca, NY (US)

(72) Inventors: Duane Hassane, New York, NY (US); Pinkal Desai, New York, NY (US); Gail J. Roboz, New York, NY (US)

(73) Assignee: Cornell University, Ithaca, NY (US)

(21) Appl. No.: 19/058,532

(22) Filed: Feb. 20, 2025

Related U.S. Application Data
(62) Division of application No. 17/311,313, filed on Jun. 4, 2021.
(60) Provisional application No. 62/776,766, filed on Dec. 7, 2018.

(51) Int. Cl. C12Q 1/6886; A61K 31/136; A61K 31/17; A61K 31/404; A61K 31/407; A61K 31/4412; A61K 31/52; A61K 31/53; A61K 31/5377; A61K 31/704; A61K 31/7048; A61K 31/7068; A61K 31/7076; C07K 16/18; G01N 33/574

(52) U.S. Cl. CPC C12Q 1/6886; A61K 31/136; A61K 31/17; A61K 31/404; A61K 31/407; A61K 31/4412; A61K 31/52; A61K 31/53; A61K 31/5377; A61K 31/704; A61K 31/7048; A61K 31/7068; A61K 31/7076; C07K 16/18; G01N 33/57426; C12Q 2600/118; C12Q 2600/156

(57) ABSTRACT

The present technology relates to methods for predicting the risk of acute myeloid leukemia (AML) in a subject prior to the onset of AML symptoms, and whether such a subject will benefit from treatment with an AML therapy. The methods disclosed herein are based on detecting the presence of mutations in the nucleic acid sequences of IDH1/2, TP53, DNMT3A, TET2, and spliceosome genes. Kits for use in practicing the methods are also provided.

### CROSS-REFERENCE TO RELATED APPLICATIONS

This application claims the benefit of and priority to U.S. Provisional Appl. No. 62/776,766, filed Dec. 7, 2018, the disclosure of which is incorporated by reference herein in its entirety.

### TECHNICAL FIELD

The present technology relates to methods for predicting the risk of acute myeloid leukemia (AML) in a subject prior to the onset of AML symptoms, and whether such a subject will benefit from or is predicted to be responsive to treatment with an AML therapy. These methods are based on detecting the presence of mutations in the nucleic acid sequences of IDH1/2, TP53, DNMT3A, TET2, and spliceosome genes in a sample obtained from a subject. Kits for use in practicing the methods are also provided.

### BACKGROUND

The following description of the background of the present technology is provided simply as an aid in understanding the present technology and is not admitted to describe or constitute prior art to the present technology.

The pathogenesis of acute myeloid leukemia (AML) is characterized by serial acquisition of somatic mutations and several genes are recurrently mutated in AML (Mardis, E. R. et al., N Engl J Med 361, 1058-1066 (2009); Ley, T. J. et al., N Engl J Med 363, 2424-2433 (2010); Ding, L. et al., Nature 481, 506-510, doi: 10.1038/nature10738 (2012)). However, it is not known when such mutations appear prior to the development of overt disease, how they evolve, and the specific risk associated with each one. Furthermore, the acquisition of AML-associated mutations has also been found in normal aging, with approximately 10% of persons greater than 65 years of age (Genovese, G. et al., N Engl J Med 371, 2477-2487 (2014); Jaiswal, S. et al., N Engl J Med 371, 2488-2498 (2014); Xie, M. et al., Nature medicine 20, 1472-1478 (2014); Coombs, C. C. et al., Cell Stem Cell 21, 374-382 e374 (2017)) having so-called “clonal hematopoiesis of indeterminate potential” (CHIP). The presence of CHIP is associated with an elevated risk of hematologic malignancies and cardiovascular disease. However, studies of CHIP to date have included very few subjects who subsequently developed AML.

Accordingly, there is an urgent need for methods that can effectively predict the risk of AML in a subject prior to the onset of AML, including determining whether specific mutations, allele burdens, or patterns of coexisting mutations would affect the risk and time-to-diagnosis of AML.

### SUMMARY

In one aspect, the present disclosure provides a method for detecting the presence of AML-associated mutations in a nucleic acid sample obtained from a subject comprising sequencing the nucleic acid sample to detect the presence of a mutation in one or more genes selected from the group consisting of SRSF2, U2AF1, and JAK2, wherein the subject does not exhibit AML symptoms or has not been diagnosed as having AML. In certain embodiments, the method further comprises detecting the presence of a mutation in SF3B1, DNMT3A, TET2, IDH2, IDH1, TP53, or any combination thereof. In another aspect, the present disclosure provides a method for detecting the presence of AML-associated mutations in a nucleic acid sample obtained from a subject comprising sequencing the nucleic acid sample to detect the presence of a mutation in one or more genes selected from the group consisting of SRSF2, DNMT3A, TET2, IDH2, IDH1, TP53, SF3B1, U2AF1, and JAK2, wherein the subject does not exhibit AML symptoms or has not been diagnosed as having AML. In any and all embodiments of the methods disclosed herein, the mutation in SRSF2, DNMT3A, TET2, IDH2, IDH1, TP53, SF3B1, U2AF1, and/or JAK2 may be a frameshift mutation, a missense mutation, a nonsense mutation, a splice site mutation, a duplication, an insertion mutation, and a deletion mutation. Additionally or alternatively, in some embodiments, the methods of the present technology further comprise detecting the presence of a mutation in one or more genes selected from the group consisting of ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, SF3B1,STAG1, STAT6, and TET1.

Additionally or alternatively, in some embodiments of the methods disclosed herein, the nucleic acid sample comprises one or more of genomic DNA, RNA, cDNA, cell-free DNA (cfDNA), cell-free RNA (cfRNA), and an exosome-associated nucleic acid. The nucleic acid sample may be a blood sample, a plasma sample, or a serum sample. In any of the preceding embodiments of the methods disclosed herein, the nucleic acid sample is sequenced using next-generation sequencing, Sanger sequencing, whole exome sequencing, targeted exome sequencing, error-corrected sequencing, augmented exome sequencing, whole genome sequencing, mRNA-seq or whole transcriptome RNA-seq.

In one aspect, the present disclosure provides a method for predicting the risk of AML in a subject prior to the onset of AML symptoms comprising detecting the presence of one or more mutations in at least one gene selected from the group consisting of SRSF2, DNMT3A, TET2, IDH2, IDH1, TP53, SF3B1, U2AF1, and JAK2 in a biological sample obtained from the subject, wherein the subject has not been diagnosed as having AML. In another aspect, the present disclosure provides a method for predicting the onset of AML symptoms in a subject that has not been diagnosed as having AML comprising detecting the presence of one or more mutations in at least one gene selected from the group consisting of SRSF2, DNMT3A, TET2, IDH2, IDH1, TP53, SF3B1, U2AF1, and JAK2 in a biological sample obtained from the subject, wherein the subject has not been diagnosed as having AML. In certain embodiments, the one or more mutations in SRSF2, U2AF1, JAK2, SF3B1, DNMT3A, TET2, IDH2, IDH1, and/or TP53 are detected using PCR (e.g., Real-time quantitative PCR (RQ-PCR), digital PCR, or reverse transcriptase PCR (RT-PCR)), Northern blots, Southern blots, microarray, dot or slot blots, in situ hybridization, electrophoresis, chromatography, mass spectroscopy, sedimentation, next-generation sequencing, Sanger sequencing, whole exome sequencing, targeted exome sequencing, error-corrected sequencing, augmented exome sequencing, whole genome sequencing, mRNA-seq or whole transcriptome RNA-seq. Additionally or alternatively, in some embodiments, the methods of the present technology further comprise detecting 2-hydroxyglutarate levels in the biological sample.

Additionally or alternatively, in some embodiments of the methods disclosed herein, the biological sample comprises one or more of genomic DNA, RNA, cDNA, cell-free DNA (cfDNA), cell-free RNA (cfRNA), and an exosome-associated nucleic acid. The biological sample may be a blood sample, a plasma sample, or a serum sample.

In any and all embodiments of the methods disclosed herein, the mutation in SRSF2, DNMT3A, TET2, IDH2, IDH1, TP53, SF3B1, U2AF1, and/or JAK2 may be a frameshift mutation, a missense mutation, a nonsense mutation, a splice site mutation, a duplication, an insertion mutation, and a deletion mutation. Additionally or alternatively, in some embodiments, the methods of the present technology further comprise detecting the presence of a mutation in one or more genes selected from the group consisting of ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, SF3B1,STAG1, STAT6, and TET1. Examples of AML symptoms include, but are not limited to, fever, fatigue, irregular heartbeat, dizziness, bone pain, frequent nosebleeds, bleeding and swollen gums, bruising on skin, loss of appetite, excessive sweating, shortness of breath, unexplained weight loss, headaches, diarrhea, menorrhagia, slurred speech, confusion, abdominal swelling, pale skin, seizures, vomiting, loss of balance, facial numbness, and blurred vision.

In any and all embodiments of the methods disclosed herein, the AML has a subtype selected from the group consisting of M0, M1, M2, M3, M4, M5, M6, M7 and M4Eo.

In one aspect, the present disclosure provides a method for selecting a subject at risk for AML for treatment with an AML therapy comprising (a) detecting the presence of one or more mutations in SRSF2, U2AF1, and JAK2 in a biological sample obtained from the subject; and (b) selecting the subject for treatment with an AML therapy, wherein the subject does not exhibit AML symptoms, and wherein the AML therapy comprises one or more of chemotherapeutic agents, FLT3 inhibitors, IDH inhibitors, Gemtuzumab ozogamicin, BCL-2 inhibitors, Hedgehog pathway inhibitors, All-trans-retinoic acid, and Arsenic trioxide. In one aspect, the present disclosure provides a method for selecting a subject at risk for AML for treatment with an AML therapy comprising (a) detecting the presence of one or more mutations in SRSF2, DNMT3A, TET2, IDH2, IDH1, TP53, SF3B1, U2AF1, and JAK2 in a biological sample obtained from the subject; and (b) selecting the subject for treatment with an AML therapy, wherein the subject does not exhibit AML symptoms, and wherein the AML therapy comprises one or more of chemotherapeutic agents, FLT3 inhibitors, IDH inhibitors, Gemtuzumab ozogamicin, BCL-2 inhibitors, Hedgehog pathway inhibitors, All-trans-retinoic acid, and Arsenic trioxide. In another aspect, the present disclosure provides a method for preventing or delaying the onset of AML symptoms in a subject at risk for AML comprising administering an effective amount of an AML therapy to the subject, wherein the subject harbors a mutation in one or more genes selected from the group consisting of SRSF2, DNMT3A, TET2, IDH2, IDH1, TP53, SF3B1, U2AF1, and JAK2, and wherein the AML therapy comprises one or more of chemotherapeutic agents, FLT3 inhibitors, IDH inhibitors, Gemtuzumab ozogamicin, BCL-2 inhibitors, Hedgehog pathway inhibitors, All-trans-retinoic acid, and Arsenic trioxide. Examples of AML symptoms include, but are not limited to, fever, fatigue, irregular heartbeat, dizziness, bone pain, frequent nosebleeds, bleeding and swollen gums, bruising on skin, loss of appetite, excessive sweating, shortness of breath, unexplained weight loss, headaches, diarrhea, menorrhagia, slurred speech, confusion, abdominal swelling, pale skin, seizures, vomiting, loss of balance, facial numbness, and blurred vision.

Additionally or alternatively, in some embodiments, the chemotherapeutic agents comprise one or more of cytarabine, an anthracycline drug (e.g., daunorubicin (daunomycin), doxorubicin, or idarubicin), cladribine, fludarabine, mitoxantrone, etoposide (VP-16), 6-thioguanine (6-TG), hydroxyurea, corticosteroid drugs (such as prednisone or dexamethasone), Methotrexate (MTX), 6-mercaptopurine (6-MP), azacitidine, and decitabine (Dacogen).

Examples of FLT3 inhibitors include, but are not limited to, midostaurin, lestaurtinib, sunitinib, sorafenib, gilteritinib, quizartinib, crenolanib, tandutinib, ponatinib, PLX3397, KW-2449, and ASP2215. Examples of IDH inhibitors include, but are not limited to AG-881 (Vorasidenib), ivosidenib, enasidenib, BAY-1436032, AGI-5198, IDH305, AGI-6780, FT-2102, HMS-101, MRK-A, and GSK321.

Examples of BCL-2 inhibitors include, but are not limited to ABT-199 (venetoclax), HA14-1, obatoclax (GX-15-070), ABT-737, GDC-0199, and ABT-263 (navitoclax). Examples of Hedgehog pathway inhibitors include, but are not limited to glasdegib, vismodegib, sonidegib, GANT-58 and GANT-61, Arsenic Trioxide, RU-SKI 43, and 5E1 monoclonal antibody.

Examples of DNMT3A mutations include, but are not limited to p.Leu731 del, p.Gly543Cys, p.Phe752Ser, p.Arg635Gly, p.Arg882Cys, p. Trp306Cys, p.Gln110AlafsTer14, p.Gly726Val, p.Phe731Leu, p.Leu905Pro, p.Arg736His, p.Gly308Arg, p.Pro904Leu, p.Arg882His, p.Ser337Leu, p.Lys766ArgfsTer13, p.Arg320Ter, p.Pro777Leu, p.Tyr533Cys, p.Arg326Cys, p.Phe755Ser, p.Arg882Ser, p. Val657Met, p. Trp313Ter, p.Arg326His, p. Tyr533Ter, p.Phe336SerfsTer9, p.Arg882Pro, p. Val328Phe, p.Arg598Ter, p.Ser770Leu, p. Thr862Ile, p.His873Pro, p.Cys557Gly, p.Arg688His, p.Gly413SerfsTer238, p. Val759TrpfsTer20, p.Phe303 SerfsTer13, p. Arg771Ter, p.Glu774Asp, p.Pro416LeufsTer235, p.Cys710AlafsTer69, p. Phe751 SerfsTer28, p.Gly293 Arg, p.Gly728Asp, p.Trp330Ter, p.Pro743His, p.Trp860Ter, p.Leu737Arg, p.Ala254HisfsTer62, p. Val830Ter, p.Gly706Glu, p.Gln485ArgfsTer166, p.Pro804Leu, p.Ala368Asp, p. Tyr528Asn, p.Phe732Ser, p.Arg736Cys, p. Tyr536Ter, p. Val296Met, p. Trp795Ter, p.Trp698Gly, p.Asp531 Asn, p. Thr503 Asnfs Ter43, p.Pro904Gln, p. Tyr735Cys, p.Phe848Ser, p.Phe794Leufs Ter4, p.Lys906Glu, p.Ile670HisfsTer43, p.Ile705Thr, p.Arg635Trp, p. Val895Met, p. Trp409Ter, p.Leu605AspfsTer7, p. Glu725Ter, p. Ser393 Valfs Ter14, p.Gly 796ValfsTer6, p.Cys537Arg, p.Phe414Val. p.Gln816AlafsTer42, p.Ala368Thr, p. Trp314Ter, p.Gly550Arg, p.Glu561Ter, p.Glu733Gly, p.Tyr908Cys, p.Pro849LeufsTer4, p.Ala910Pro, p.Trp860Arg, p.Cys666TrpfsTer39, p.Arg458GlyfsTer193, p. Trp305Gly, p.Asn797ThrfsTer5, p.Ala368Val, p. Val502AspfsTer43, p.Ile780Thr, p.Met852IlefsTer29, p.Pro849Ser, p.Trp601Ter, p.Met761Val, p.Asn797Lys, p.Arg899Cys, p.Arg301Trp, and p.Arg749Gly.

Examples of IDH1 mutations include, but are not limited to p.Arg132Cys or p.Arg132Gly, and examples of IDH2 mutations include, but are not limited to p.Arg140Gln, p.Arg140His, and p.Arg140Trp. Examples of JAK2 mutations include, but are not limited to p. Val617Phe, p.Gly48Glu, and p.Glu814Gly.

Examples of SF3B1 mutations include, but are not limited to p.Arg625Leu, p.Lys790Glu, p.Gly742Asp, p.Lys700Glu, p.Ala263Val, p.Lys666Asn, p.Ala744Val, p.His662Asp, and p.Arg625Cys. Examples of SRSF2 mutations include, but are not limited to p.Pro95His, p.Pro95Leu, p.Pro95Arg, and p.Pro95Thr. Examples of U2AF1 mutations include, but are not limited to p.Arg156His, p.Gln157Arg, p. Tyr158dup, and p.Gln157Pro.

Examples of TET2 mutations include, but are not limited to p.Gln644Ter, p.Gln1510Ter, p.Ser167PhefsTer4, p.His1912Tyr, p.Gln180Ter, p.Gln1523Ter, p.Pro1356_Glu1357del, p.Glu1318Gly, p.Cys1263Arg, p.Glu1874Gln, p.Asn140Ser, p.Asn 140ThrfsTer8, p.Gln892Ter, p.Ile1105TyrfsTer25, p.Leu1780SerfsTer38, p. Val291GlyfsTer2, p.Asp302ValfsTer6, p.Gln706Ter, p. Val291 TrpfsTer2, p.Glu320AsnfsTer27, p.His786LeufsTer27, p.Leu1515AlafsTer62, p.Asn1489MetfsTer82, p.Arg1451GlyfsTer7, p.Met695CysfsTer5, p.Leu1151Pro, p. Val647TrpfsTer53, p.Tyr1337Ter, p.Cys1378Tyr, p.Ala727HisfsTer23, p.Glu1320ArgfsTer43, p. Trp954LeufsTer18, p. Tyr1148LeufsTer9, p.His1881Tyr, p. Val 1900Gly, p.Leu1511TrpfsTer60, p.Glu537Ter, p.Gln764Ter, p.Asp1427ValfsTer22, p.Leu920SerfsTer2, p.Gln740Ter, p.Pro1594GInfsTer37, p.Ile1873Asn, p.Gly 1361Asp, p.Leu500Ter, p.Gly773Ter, p.Gln321Ter, p.Gln745Ter, p.Asp1858SerfsTer10, p.Ala1158Val, p.Ser1494Ter, p.Cys1875Gly, p.Leu719Ter, p.Ala1876Val, p.Gln705Ter, p.Ser1870Leu, p.His1386Asp, p.Gln1414His, p.Asn442LysfsTer19, p.Lys664Glu, p.Arg1452Ter, p.Ser1898Pro, p.Cys1378Arg, p.Gln734Ter, p.His1904Leu, p. Thr556AsnfsTer11, p.Cys1263Tyr, p.Pro1644HisfsTer51, p. Gly641 ArgfsTer40, p.Glu1879Val, p.His1904Arg, p.Ala1512Val, p.His1904Gln, p.Met1333TyrfsTer6, p.Ile1160TyrfsTer2, p.Arg1359Ser, p.Asn258MetfsTer35, p.Lys1299Ter, p.Ala1174LysfsTer53, p.Asn442Thrfs Ter5, p.Arg1516Ter, p.Gly 1275Arg, p. Ile1873Thr, p.Trp1847Ter, p.Thr229AsnfsTer25, p. Tyr1294Ter, p.Arg1712Ter, p. Tyr1902Cys, p.Leu200ThrfsTer2, p.Gly 1282Cys, p.Asp1376Gly, p. Val 1056LeufsTer10, p.Cys973LeufsTer3, p.Gln1547LeufsTer19, and p.Leu1276TrpfsTer87.

Examples of TP53 mutations include, but are not limited to p. Leu93 ArgfsTer30, p.Arg337Cys, p.Gln167Ter, p.Leu145Pro, p.Lys321Ter, p. Gln100Ter, p.Arg248Trp, p.Arg273His, p.Ala161Thr, p.Arg175Gly, p.Tyr163His, p. Tyr236His, p.Cys275Tyr, p.Ile195Ser, p.Pro128LeufsTer42, p.Tyr220Cys, p. Val272Met, p.Cys242Tyr, p.Asn29ThrfsTer15, p.Arg333 ValfsTer12, p. Met246Ile, p.Pro278His, p.Asn239Ser, p. Val143Met, p.Arg175His, p. Thr155Asn, p. Tyr234Cys, p.Phe109SerfsTer14, and p.Cys275Phe.

### BRIEF DESCRIPTION OF THE DRAWINGS

FIGS. 1A-1C: Spectrum of mutations seen at baseline years prior to the diagnosis of AML alongside matched controls. FIG. 1A: OncoPrint summarizing mutated genes AML cases (N=189; left) vs. controls (N=183; right). For each gene (rows), pathogenic mutations (dark squares) are indicated vs. variants of unknown significance (VUS; light squares). Vertical barplots (right of each OncoPrint) indicate the number of participants in whom the gene is mutated (#mut) in each group. Horizontal barplots (top of each OncoPrint) indicate the number of mutated genes (#mut) in each participant. For the AML controls, the time to AML and age (≥65 years vs. <65 years) are shown. For the controls, the time of follow up and age (≥65 years vs. <65 years) are shown. FIG. 1B: Chord plot indicates co-mutations between genes in AML cases (n=133) alongside controls (n=68). The arc length is directly proportional to the percent of instances of co-mutation. “Other” includes genes not recurrently mutated in AML. FIG. 1C: Violin plots indicating the distribution of the number of mutated genes per participant in AML cases vs. controls. The width of violin bouts is proportional to the number of participants harboring the number of mutated genes indicated on the vertical axis. Top row (left) indicates the number of mutated genes per participant in AML cases (median 1, range 0-8, n=133/189 mutated) vs. controls (median 0, range 0-4, n=68/183 mutated). Top row (right) indicates the number of genes with pathogenic mutations per participant in AML cases (median 1, range 0-8, n=130/189 mutated) vs. controls (median 1, range 0-4, n=62/183 mutated). The bottom row further stratifies differences in mutation number into age groups (<65 years vs. ≥65 years). Bottom row (first panel) indicates the number of mutated genes per participant (<65 years; n=158) in AML cases (median 1, range 0-4, n=158) vs controls (median 0, range 0-2, n=20/77 mutated). Bottom row (second panel) indicates the number of mutated genes per participant (≥65 years; n=214) in AML cases (median 2, range 0-8, n=88/108 mutated) vs. controls (median 0, range 0-4, n=48/106 mutated). Bottom row (third panel) indicates the number of genes with pathogenic mutations per participant (<65 years; n=158) for AML cases (median 1, range 0-3, n=43/81 mutated) vs. controls (median 0, range 0-2, n=18/77 mutated). Bottom row (fourth panel) indicates the number of genes with pathogenic mutations per participant (≥65 years; n=214) for AML cases (median 1, range 0-6, n=87/108 mutated) vs. controls (median 0, range 0-4, n=44/106). *** P<0.001, Wilcoxon test.

FIGS. 2A-2C: Time to AML diagnosis is influenced by mutation status. Cumulative incidence of AML diagnoses (cumulative event; vertical axis) as a function of time (years to AML diagnosis) is shown. Participants include AML cases only at baseline (N=189) with any mutated gene (n=133/189) vs. no mutations (n=56/189) (FIG. 2A); mutations in high risk genes associated with AML vs. participants with no mutations in these genes (DNMT3A, n=71/189; TET2, n=48/189; TP53, n=23/189; IDH1 or IDH2, n=15/189; spliceosome, n=27/189) in addition to RUNX1 (n=3/189) (FIG. 2B). Data on RUNX1 are provided because all participants with a RUNX1 (n=3) mutation rapidly developed AML (<2 years) although significance was not achieved due to the few participants mutated in RUNX1 within the cohort; or AML cases harboring zero mutated genes (n=85/189), 1 mutated gene (n=56/189), or 2 or more mutated genes (2+) (n=48/189) in significant high risk genes associated with development of AML (FIG. 2C). P-values are shown for the log rank test.

FIGS. 3A-3C: Mutations pose AML risk irrespective of the variant allele fraction. FIG. 3A: Histogram indicating the maximum allelic fraction for mutations in each gene shown per participant at baseline (DNMT3A, n=106 (71 AML cases, 35 controls); TET2, n=59 (48 AML cases, 11 controls); TP53, n=23 (23 AML cases, 0 controls); SRSF2, n=14 (13 AML cases, 1 control); IDH2, n=13 (12 AML cases, 1 control); JAK2, n=13 (11 AML cases, 2 controls); SF3B1, n=13 (11 AML cases, 2 controls); U2AF1, n=7 (7 AML cases, 0 controls); IDH1, n=3 (3 AML cases, 0 controls); RUNX1, n=3 (3 AML cases, 0 controls). The proportion of AML cases and controls is shown for each bin in the histogram (bin width=5% allelic fraction). FIG. 3B: Receiver operating characteristic (ROC) curves indicating the % true positive rate (vertical axis) vs. the % false positive rate (horizontal axis) of mutations to detect AML cases. The curves indicate performance at decreasing allelic fraction (%) with 1%, 2.5%, 5%, and 10% indicated specifically (filled black circles). Performance is shown for mutations in any gene significantly associated with the AML case group (left plot; DNMT3A, TET2, IDH1, IDH2, SRSF2, SF3B1, U2AF1, TP53; n=167 [120 AML cases, 47 controls]) or the same set of genes excluding DNMT3A (n=96; 82 AML cases, 14 controls) (right plot). FIG. 3C: Fold change in variant allele fraction (VAF) per year influences kinetics of AML diagnosis for TP53 (n=7) and IDH2 (n=8). Time to AML (years; vertical axis) is plotted against fold change in VAF as determined by comparing the VAF at baseline vs. the VAF at year 1 or year 3. Regression line is shown for each mutation in each gene. R² and p-values are indicated for linear regression. Data on RUNX1 are provided since all participants with a RUNX1 (n=3) mutation rapidly developed AML (<2 years). DNMT3A, n=16; TET2, n=13; SRSF2, n=4; JAK2, n=7; SF3B1, n=5; U2AF1, n=4. Data points shown are AML cases with available serial samples showing a significant increase in VAF between baseline and year 1 or year 3.

FIG. 4: Clonal evolution towards AML in selected patients. Clonal composition and evolution are shown for four selected examples of participants who were evaluable serially (cases A, B, C, and D). Peripheral blood was sampled at baseline and years 1 or 2. The horizontal axis indicates time (years). The vertical axis indicates the VAF where the maximum possible VAF is 1 (100%). Mutated genes are shown at each time point as indicated on the line chart. Time of AML diagnosis (AML Dx) relative to baseline is indicated by the vertical dotted line. Case A: An IDH2 mutation (8% VAF) is present at baseline at lower VAF and persists at year 1 at 13% VAF with an acquired NPM1 type A mutation at 14% VAF. AML diagnosis occurs <30 days after the year 1 sample. Case B: DNMT3A mutation remained stable from baseline to year 1 follow up. VAFs of JAK2 and SF3B1 increased from 5% to 24% before AML diagnosis at 4.3 years from baseline. Case C: Clonal expansion of TP53 from 3% to 21% with acquired SRSF2 and CUX1 mutations between baseline and year 3 follow up. TET2 remains relatively stable. AML diagnosed at 4.4 years from baseline. Case D: Low VAF mutations in IDH2 and TET1 expand by year 3 along with acquisition of SRSF2 in the presence of a relatively stable DNMT3A mutation. AML diagnosis occurs 6.6 years from baseline.

FIG. 5: Coverage across gene regions for top mutated genes. Representative images for coverage of recurrently mutated genes in the cohort at the median depth of coverage of 2000× are shown. For each gene, coverage across pertinent exons approaches or exceeds 2000× including important regions of driver genes such as FLT3. CEBPA also achieved >500× median coverage across its single coding exon. Median NPM1 exon 12 coverage was relatively lower (˜280×) potentially resulting in more false negatives. However, given the zero background rate of 4 nucleotide insertions in the NPM1 insertion hotspot, this lower coverage maintains >80% power to detect NPM1 insertions to a VAF >1% (1-sample binomial power calculation; background mutation rate of 0.1%, alpha=0.01).

FIG. 6: Spectrum and co-mutation pattern of pre-leukemic mutations in 1 AML in the complete cohort. OncoPrint for all participants in the study at baseline. AML cases (N=189) are represented in the left panel and controls (N=183) in the right panel. Each row represents a gene and each column corresponds to a participant in the study. Bar plots indicate the number of mutations per patient (top bar plot), and the number of patients with mutations in each gene (side bar plot). For each patient, bottom panels show: time to AML diagnosis (Time to AML) for the cases or last follow up for the controls (Follow up time) and age at diagnosis (Age). Dark grey, patients older or equal than 65 years old; light grey, patients younger than 65 years old. Alterations classified as variant of unknown significance (VUS) or pathogenic (pathogenic) according to the criteria specified in the methods described herein. All analyzed genes are included.

FIGS. 7A-7B: Mutation variant analysis summary in the AML case group. A total of 319 variants were identified in AML case group (n=133). Missense mutations accounted for 62.6% (199 mutations) followed by deletions (11.3%; 37 mutations), insertions (7.5%; 24 mutations), and 1.8% CNVs (6 total found exclusively in AML cases, denoted as “Complex” or “Other”). FIG. 7A: Top row: bar plots enumerate variant types (left); variant classification (middle); SNV substitution (right). SNV, single nucleotide variant; INS, insertion; DEL, deletion. Bottom row: number of variants per sample (left); variant classification summary; and frequently mutated genes (right). N indicates number of mutations per sample. FIG. 7B: Top row: boxplots indicating overall distribution of six different types of conversions (left) or transitions (Ti) and transversions (Tv) (right). Bottom row: Stacked bar plot shows the fraction of conversions per sample (bottom panel).

FIGS. 8A-8B: Mutation variant analysis summary in the control group. A total of 91 variants were identified in the control group (n=68). Missense mutations accounted for 60.4% of mutations (55 mutations total), followed by deletions (16.4%; 15 mutations), insertions (6.5%; 6 mutations). As with the AML cases, the most common single-nucleotide change was a cytosine-to-thymine (C>T) transition occurring in CpG context for both groups (FIGS. 5, 6, 7A-7B), a lesion and context associated with age-related mutagenesis and consistent with other reports. FIG. 8A: Top row: bar plots enumerate variant types (left); variant classification (middle); SNV substitution (right). SNV, single nucleotide variant; INS, insertion; DEL, deletion. Bottom row: number of variants per sample (left); variant classification summary; and frequently mutated genes (right). N indicates number of mutations per sample. FIG. 8B: Top row: boxplots indicating overall distribution of six different types of conversions (left) or transitions (Ti) and transversions (Tv) (right). Bottom row: Stacked bar plot shows the fraction of conversions per sample (bottom panel).

FIG. 9: Distribution of point mutation types 1 in the AML and control group. Relative contribution of base substitutions when focusing on missense mutations enumerated in the AML case group (n=199; FIG. 5) and the control group (n=55; FIG. 6). The majority of C>T transitions occur in CpG context for both groups, suggesting acquisition from misrepair deamination of 5-methylcytosine. The most common single-nucleotide change was a cytosine-to-thymine (C>T) transition occurring in CpG context for both groups, a lesion and context associated with age-related mutagenesis and consistent with other findings.

FIGS. 10A-10B: SNV signature analysis and transcribed strand bias in the AML case and control groups. A broader analysis of base substitution was performed taking into account the bases immediately upstream and downstream of the mutated base providing mutation context information (panel A). An elevated rate of spontaneous deamination of 5-methyl-cytosine occurring predominantly NpCpG trinucleotide is consistent with reports of mutation patterns in AML. Of note, a similar pattern is observed for AML cases and controls, which suggests common underlying mutation processes in the two groups most likely driven by aging. Further analysis of the mutation pattern shows preference for certain substitutions in the transcribed strand over the untranscribed strand suggesting additional mutational processes driven by transcription-coupled repair (TCR), a nucleotide excision repair (NER) process that has been shown to decrease in efficiency with normal aging. While strand bias suggesting TCR approached significance (P<0.05) for the AML group, the control group demonstrated the same trend toward strand bias also suggesting TCR but did approach significance because of lower number of cases. FIG. 10A: Trinucleotide context of C>A, C>G, C>T, T>A, T>C, and T>G point mutations is shown among non-synonymous point mutations in the AML case and control groups. For each context, the stacked bar chart indicates mutations occurring on the untranscribed strand (filled) vs the transcribed strand (not filled). FIG. 10B: Transcribed vs. untranscribed strand bias (log 2 scale) is indicated for each type of point mutation type in the AML case (n=199 variants, n=110 participants) and control group (n=55 variants, n=43 participants). Significant differences are indicated by asterisks (*) using the Poisson test. * P<0.05 as implemented in the MutationPatterns R package.

FIGS. 11A-11C: VAF cutoff tables for specificity and sensitivity. Analysis of sensitivity and specificity showed that the false positive rate for individuals bearing mutations in TP53, SRSF2, IDH2, SF3B1 or U2AF1 in the 1-2% VAF range is less than 1%. Table of VAF cut offs for the significantly mutated genes producing no greater than a 1% false positive rate (FIG. 11A), 5% (FIG. 11B) or 10% (FIG. 11C) while maximizing sensitivity. False positives represent controls misclassified as AML cases.

FIGS. 12A-12B: Variation in VAF over time in recurrently mutated genes. Mutations in recurrently mutated genes in patients with serial samples were tracked over time. Generally, no changes were observed within the first year in AML cases or controls. At 3 years, however, the AML cases demonstrated elevations in VAF. In contrast, the VAF in the controls group remained mostly stable up to 3 years. FIG. 12A: Comparison of allelic fractions at baseline (horizontal axis) and after 3 years of follow up (vertical axis). FIG. 12B: Comparison of allelic fractions at baseline (horizontal axis) and after 1 year of follow up (vertical axis). The diagonal represents no change in VAF. Individual AML cases mutated in the gene are indicated with dots (AML case) or boxed dots (control). The maximum VAF was selected when participants harbored more than 1 mutation.

FIGS. 13A-13B: Persistence of mutations detected at baseline for patients with serial samples. The persistence of mutations was assessed in evaluable cases and controls with serial samples available at baseline and 1-year or 3-year follow-up. 89% of the variants found at baseline persist in follow-up peripheral blood draws, indicating that most mutations observed in this study were stable. This stability was maintained irrespective of whether the participant's blood sample was derived from the case or control group: e.g., 90% of DNMT3A mutations (n=27/30) were stable in the control group vs. 87% (n=81/93) in the AML cases. VAF threshold for follow up time point was set to 0.8% to capture persistence in mutations near the 1% VAF cutoff. FIG. 13A: Persistence or loss of mutations per gene in the entire cohort expressed as a percentage of total mutations found per gene. Number of mutations that persisted over time out of the total of mutations are indicated for each gene. FIG. 13B: Persistence or loss of mutations per gene and group, AML case (left panel) or control (right panel) are expressed as a percentage of total mutations found per gene and group. Number of mutations that persisted over time out of the total of mutations are indicated for each gene and group. Fractions indicate the number of persisting mutations/total number of mutations.

FIGS. 14A-14E: Mapping of coding alterations to protein domains. Mapping of coding alterations to protein domains for recurrently mutated genes in the cohort: DNMT3A, TET2, TP53, SRSF2, IDH2, JAK2, SF3B1, U2AF1, ASXL1, IDH1 and RUNX1. For each gene, mutations identified in the AML case and control groups are plotted except for genes that were not mutated in the control group: TP53, U2AF1 and RUNX1. Mutations are classified as Missense, In-frame, and Truncating. Mutations in IDH2, SRSF2, JAK2, SF3B1 and U2AF1 occurred in positions R140, P95, V617, K700 and Q157, respectively. Other point mutations were detected in the HEAT domain of SF3B1 in close proximity to K700 or in the zinc finger domain of U2AF1 in close proximity to Q157. All of these positions are known hotspots for the aforementioned genes and highly associated with hematological malignancies and especially AML. Mutations in DNMT3A were localized in exons 8-23. 80% of the variants detected in the gene corresponded to SNV with missense mutations in the R882 position accounting for 22% of the SNVs. The second most common alteration were truncating variants affecting all the functional domains. SNVs demonstrated an overall tendency to occur in functional domains whereas truncating mutations occurred in the N-terminal half of the protein. Missense mutations comprised 52% of the observed mutations in TET2 with the majority of the missense SNVs being confined to oxygenase domain of TET2 (Tet2_JDP). Truncating mutations were distributed across coding exons. Mutations in ASXL I were predominantly found in exon 13, with the most common type of alteration being non-sense SNV. Truncations in the carboxy-terminus or premature stop variants have a disrupting effect whereas missense variants have an unknown significance. Mutations in TP53 were also found distributed along the gene. SNVs were concentrated in the DNA binding domain with additional variations in tetramerization and transactivating domains. Truncating mutations occurred throughout. The vast majority of mutations were missense SNV (75%) including known hotspots with few frameshift deletions, consistent with the mutation pattern observed for mutations in TP53 in human cancer.

FIG. 15: Spatial clustering of TP53 point mutations in 3-D structure. Despite having distant amino acid positions, mutations within TP53 across the AML cases were specially localized when mapped to the tertiary structure of the protein. Positions of SNVs are indicated by spheres. Clustering was performed using mutation3D. Two distinct clusters were identified each encompassing known regions involved in DNA contact (e.g., R248 and R273) and structural support (e.g., R175). Cluster 1 (spheres) indicates alterations in amino acid positions 239, 242, 248 (4 participants), 272 (2 participants), 273,275 (2 participants), and 278 (P=0.02, non-parametric bootstrap). Cluster 2 (boxed spheres) indicates mutations in positions 161, 175, 195 (2 participants), and 234 (P=0.06, non-parametric bootstrap). PDB accession number is 2XWR.

FIG. 16: Distribution of mutations in protein domains (Pfam) in the AML case and control group. Number of genes (nGenes; vertical axis) is plotted vs. number of mutations (nMuts; horizontal axis). AML case samples: Top mutated Pfam domains include PTZ00435 (isocitrate dehydrogenase IDH1 Arg132 and IDH2 Arg140), AdoMet_MTases (AdoMet methytransferase; DNMT3A), and P53 (DNA binding domain, TP53). Control samples: Top mutated Pfam domains include Dem (DNA cytosine methyltransferase; DNMT3A), zf-C3HC4_2 (Zinc finger, C3HC4 type RING finger; CBL and CBLC), and Pkinase_Tyr (tyrosine kinase domain; JAK1 Lys696 and JAK2 Val617).

FIG. 17: Summary of clinical parameters. Summary of clinical parameters for the entire cohort, AML cases or control participants (mutated, non-mutated and in total). Mean and standard deviation is provided for each variable, time to AML, age, hematocrit, white blood cell (WBC), hemoglobin and platelet counts.

FIG. 18: Mutated cases in case and control group. Number of participants with mutations in selected genes within the AML case or control groups overall and for patients <65 or ≥65 years of age.

FIG. 19: Mutated cases in AML case group with time to AML less or greater than 5 years. Number of participants with mutations in selected genes within the AML case group with 5, <5 or ≥5 years to the diagnosis of AML.

### DETAILED DESCRIPTION

The present disclosure demonstrates that the most significant mutations associated with increased odds of AML included those in TP53, IDH1/2, spliceosome (SRSF2, SF3B1, U2AF1), TET2, and DNMT3A. Subjects with baseline TP53 or DNMT3A mutations were more likely to develop AML within 5 years. Participants with mutations in the RUNX1 gene all developed AML within 2 years from baseline. The time-to-AML was inversely correlated with increasing VAF in TP53, IDH2, and possibly DNMT3A mutations. The present disclosure provides a set of genes with high AML penetrance including TP53, IDH2, SRSF2, SF3B1, and U2AF1, and further demonstrates that for specific genes (i.e., TP53, IDH2, SF3B1, SRSF2, U2AF1), a VAF cutoff correlating with a false-positive fraction of less than 1% could be achieved. These results demonstrate that detectable mutations likely arising from pre-leukemic clones are present in peripheral blood of individuals at a median of 9.8 years prior to the diagnosis of AML. The presence of mutations in TP53 or multiple mutations was associated with increased odds of developing AML within 5 years. Serial samples also revealed the stepwise acquisition of mutations leading to AML. Mutations in genes commonly associated with clonal hematopoiesis such as DNMT3A and TET2 were maintained over time, while new dominant sub-clones arose in genes such as NPM1, TP53 and SRSF2 preceded the development of AML. The ability to detect and identify high-risk mutations suggests that monitoring strategies for patients and clinical trials of potentially preventative interventions can be proactively considered.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, an “alteration” of a gene or gene product (e.g., a marker gene or gene product) refers to the presence of a mutation or mutations within the gene or gene product, e.g., a mutation, which affects the quantity or activity of the gene or gene product, as compared to the normal or wild-type gene. The genetic alteration can result in changes in the quantity, structure, and/or activity of the gene or gene product in a cancer tissue or cancer cell, as compared to its quantity, structure, and/or activity, in a normal or healthy tissue or cell (e.g., a control). For example, an alteration which is associated with AML, or predictive of responsiveness to anti-AML therapeutics, can have an altered nucleotide sequence (e.g., a mutation), amino acid sequence, chromosomal translocation, intra-chromosomal inversion, copy number, expression level, protein level, protein activity, in a cancer tissue or cancer cell, as compared to a normal, healthy tissue or cell. Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene. In certain embodiments, the alterations are associated with a phenotype, e.g., a cancerous phenotype (e.g., one or more of AML risk, AML progression, or responsiveness to AML therapy). In one embodiment, the alteration is associated with one or more of: a genetic risk factor for AML, a positive treatment response predictor, a positive prognostic factor, a negative prognostic factor, or a diagnostic factor.

As used herein, the terms “amplify” or “amplification” with respect to nucleic acid sequences, refer to methods that increase the representation of a population of nucleic acid sequences in a sample. Nucleic acid amplification methods are well known to the skilled artisan and include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA) (TwistDx, Cambridge, UK), transcription mediated amplification, signal mediated amplification of RNA technology, loop-mediated isothermal amplification of DNA, helicase-dependent amplification, single primer isothermal amplification, and self-sustained sequence replication (3SR), including multiplex versions or combinations thereof. Copies of a particular nucleic acid sequence generated in vitro in an amplification reaction are called “amplicons” or “amplification products.”

“Bait”, as used herein, is a type of hybrid capture reagent that retrieves target nucleic acid sequences for sequencing. A bait can be a nucleic acid molecule, e.g., a DNA or RNA molecule, which can hybridize to (e.g., be complementary to), and thereby allow capture of a target nucleic acid. In one embodiment, a bait is an RNA molecule (e.g., a naturally-occurring or modified RNA molecule); a DNA molecule (e.g., a naturally-occurring or modified DNA molecule), or a combination thereof. In other embodiments, a bait includes a binding entity, e.g., an affinity tag, that allows capture and separation, e.g., by binding to a binding entity, of a hybrid formed by a bait and a nucleic acid hybridized to the bait. In one embodiment, a bait is suitable for solution phase hybridization.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

As used herein, the term “clonal hematopoiesis of AML potential (CHAP)” refers to mutations associated with an increased risk of AML that can be detected at a VAF cutoff correlating with a false-positive fraction of less than 1% in controls.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a non-tumor sample from the same or a different subject.

“Detecting” as used herein refers to determining the presence of a mutation or alteration in a nucleic acid of interest in a sample. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

“Detectable label” as used herein refers to a molecule or a compound or a group of molecules or a group of compounds used to identify a nucleic acid or protein of interest. In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable labels include but are not limited to spectroscopic, photochemical, biochemical, immunochemical, electromagnetic, radiochemical, or chemical means, such as fluorescence, chemifluorescence, or chemiluminescence, or any other appropriate means.

As used herein, the term “effective amount” refers to a quantity sufficient to achieve a desired therapeutic and/or prophylactic effect, e.g., an amount which results in the prevention of, or a decrease in a disease or disorder or one or more signs or symptoms associated with a disease or disorder (e.g., AML). In the context of therapeutic or prophylactic applications, the amount of a composition administered to the subject will depend on the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder. As used herein, a “therapeutically effective amount” of a compound refers to compound levels in which the physiological effects of a disease or disorder are, at a minimum, ameliorated.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N. Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” are used interchangeably and refer to an individual organism, a vertebrate, a mammal, or a human. In certain embodiments, the individual, patient or subject is a human.

As used herein, the term “library” refers to a collection of nucleic acid sequences, e.g., a collection of nucleic acids derived from whole genomic, subgenomic fragments, CDNA, cDNA fragments, RNA, RNA fragments, cell-free DNA, or a combination thereof. In one embodiment, a portion or all of the library nucleic acid sequences comprises an adapter sequence. The adapter sequence can be located at one or both ends. The adapter sequence can be useful, e.g., for a sequencing method (e.g., an NGS method), for amplification, for reverse transcription, or for cloning into a vector.

The library can comprise a collection of nucleic acid sequences, e.g., a target nucleic acid sequence (e.g., a nucleic acid sequence associated with AML), a reference nucleic acid sequence, or a combination thereof. In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject. In one embodiment, the subject is a human that is at risk for AML.

A “library nucleic acid sequence” refers to a nucleic acid molecule, e.g., a DNA, RNA, or a combination thereof, that is a member of a library. Typically, a library nucleic acid sequence is a DNA molecule, e.g., genomic DNA, cell-free DNA, or cDNA. In some embodiments, a library nucleic acid sequence is fragmented, e.g., sheared or enzymatically prepared, genomic DNA. In certain embodiments, the library nucleic acid sequences comprise sequence from a subject and sequence not derived from the subject, e.g., adapter sequence, a primer sequence, or other sequences that allow for identification, e.g., “barcode” sequences.

The term “multiplex PCR” as used herein refers to amplification of two or more PCR products or amplicons which are each primed using a distinct primer pair.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 103, 104, 105 or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

“Probe” as used herein refers to a nucleic acid that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a target nucleic acid. Probes are typically at least about 10, 15, 20, 25, 30, 35, 40, 50, 60, 75, 100 nucleotides or more in length.

As used herein, a “sample” or a “biological sample” refers to a substance that is being assayed for the presence of a mutation in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%).

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include 90, 95, 98, and 99%.

The term “stringent hybridization conditions” as used herein refers to hybridization conditions at least as stringent as the following: hybridization in 50% formamide, 5×SSC, 50 mM NaH2PO4, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “target sequence” and “target nucleic acid sequence” refer to a specific nucleic acid sequence to be detected and/or quantified in the sample to be analyzed.

As used herein, the terms “treat,” “treating” or “treatment” refer, to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) state of disease, amelioration or palliation of the disease state, diminishing rate of or time to progression, and remission (whether partial or total).

### AML

Acute myeloid leukemia (AML), also called acute nonlymphocytic, granulocytic, myelocytic, myeloblastic, or myeloid leukemia, is a disease in which cancer cells develop in the blood and bone marrow. The cancer develops from two main types of immature white blood cells that normally develop into mature granulocytes or monocytes. The result is a malignancy characterized by the accumulation in blood and bone marrow of abnormal hematopoietic progenitors and disruption of normal production of erythroid, myeloid, and/or megakaryocyte cell lines. AML can be subdivided morphologically into specific types depending on which cell lines are involved. AML subtypes (MO-M7) are determined by cell morphology with particular subtypes such as M3 (acute promyelocytic leukemia or APL) having a more favorable outcome: MO: undifferentiated large granular; M1 and M2: acute myeloblastic; M3: acute promyelocytic; M4: myelomonocytic; M5: monocytic; M6: erythroleukemia; M7: megakaryocyte; and M4Eo: eosinphils.

Current AML therapy regimens generally involve two stages: Initial treatment (“induction therapy”) for AML is aimed at eradicating the leukemic clone to reestablish normal hematopoiesis, and post-remission therapy. AML treatment generally involves chemotherapy, and sometimes involves radiation therapy to relieve AML-induced bone pain. For patients who have relapses or have AML that does not respond to other treatment, bone marrow transplantation (“BMT”) may be required, and can often increase survival.

Certain chromosomal abnormalities are routinely used to determine prognosis in adult AML patients, including t (8;21), t (15;17) or inv (16) suggestive of better prognosis; t (9;11) used to classify patients at intermediate risk; and inv (3),-5/del (5q),-7/del (7q), t (6;9), abnormalities involving 11q23, or a complex karyotype (three or more cytogenetic aberrations) used to classify patient as being at high risk (Valk et al., New England J Medicine, 350 (16): 1617-1628; Bullinger et al., New England J. Medicine, 350 (16): 1605-1616 (2004)). However a significant proportion of AML patients do not exhibit such genetic abnormalities. These patients are termed the “normal karyotype” subset of AML patients, and there is currently no consensus for either risk stratification or optimal treatment regimen for this group.

Methods for Detecting Polynucleotides Associated with AML

Polynucleotides associated with AML may be detected by a variety of methods known in the art. Non-limiting examples of detection methods are described below. The detection assays in the methods of the present technology may include purified or isolated DNA (genomic or cDNA), RNA or protein or the detection step may be performed directly from a biological sample without the need for further DNA, RNA or protein purification/isolation.

### Nucleic Acid Amplification and or Detection

Polynucleotides associated with AML can be detected by the use of nucleic acid amplification techniques that are well known in the art. The starting material may be genomic DNA, cDNA, RNA or mRNA. Nucleic acid amplification can be linear or exponential. Specific variants or mutations may be detected by the use of amplification methods with the aid of oligonucleotide primers or probes designed to interact with or hybridize to a particular target sequence in a specific manner, thus amplifying only the target variant.

Non-limiting examples of nucleic acid amplification techniques include polymerase chain reaction (PCR), reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7 (suppl 2): S11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

Primers: Oligonucleotide primers for use in amplification methods can be designed according to general guidance well known in the art as described herein, as well as with specific requirements as described herein for each step of the particular methods described. In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tm of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

Probes: Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif, 1987); Young and Davis, PNAS. 80:1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29: E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl) maleimide; anthranilamide; Black Hole Quencher™ (BHQ™) dyes (biosearch Technologies); BODIPY dyes: BODIPY® R-6G, BOPIPY® 530/550, BODIPYR FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with SO₃ instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers, baits, or probes can be designed so that they hybridize under stringent conditions to the allelic variants of SRSF2, DNMT3A, TET2, IDH2, IDH1, TP53, SF3B1, U2AF1, or JAK2, and optionally ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, or TET1 but not to the respective wild-type nucleotide sequences. Primers, baits, or probes can also be prepared that are complementary and specific for the wild-type nucleotide sequence of SRSF2, DNMT3A, TET2, IDH2, IDH1, TP53, SF3B1, U2AF1, or JAK2, and optionally ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, or TET1, but not to any one of the corresponding allelic variants described herein.

In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, for example, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLID sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454TM GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 4541. system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added poly A tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLID™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### AML Screening Methods of the Present Technology

Disclosed herein are methods and assays that are based on the principle that assaying cell populations within a biological sample (e.g., blood, plasma, serum) for the presence of one or more alterations in SRSF2, DNMT3A, TET2, IDH2, IDH1, TP53, SF3B1, U2AF1, or JAK2, and optionally ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, or TET1, is useful in determining whether a patient will benefit from or will respond to treatment with a particular therapeutic agent.

In one aspect, the present disclosure provides a method for detecting the presence of AML-associated mutations in a nucleic acid sample obtained from a subject comprising sequencing the nucleic acid sample to detect the presence of a mutation in one or more genes selected from the group consisting of SRSF2, U2AF1, and JAK2, wherein the subject does not exhibit AML symptoms or has not been diagnosed as having AML. In certain embodiments, the method further comprises detecting the presence of a mutation in SF3B1, DNMT3A, TET2, IDH2, IDH1, TP53, or any combination thereof. In another aspect, the present disclosure provides a method for detecting the presence of AML-associated mutations in a nucleic acid sample obtained from a subject comprising sequencing the nucleic acid sample to detect the presence of a mutation in one or more genes selected from the group consisting of SRSF2, DNMT3A, TET2, IDH2, IDH1, TP53, SF3B1, U2AF1, and JAK2, wherein the subject does not exhibit AML symptoms or has not been diagnosed as having AML. In any and all embodiments of the methods disclosed herein, the mutation in SRSF2, DNMT3A, TET2, IDH2, IDH1, TP53, SF3B1, U2AF1, and/or JAK2 may be a frameshift mutation, a missense mutation, a nonsense mutation, a splice site mutation, a duplication, an insertion mutation, and a deletion mutation. Additionally or alternatively, in some embodiments, the methods of the present technology further comprise detecting the presence of a mutation in one or more genes selected from the group consisting of ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, SF3B1, STAG1, STAT6, and TET1.

Additionally or alternatively, in some embodiments of the methods disclosed herein, the nucleic acid sample comprises one or more of genomic DNA, RNA, cDNA, cell-free DNA (cfDNA), cell-free RNA (cfRNA), and an exosome-associated nucleic acid. The nucleic acid sample may be a blood sample, a plasma sample, or a serum sample. In any of the preceding embodiments of the methods disclosed herein, the nucleic acid sample is sequenced using next-generation sequencing, Sanger sequencing, whole exome sequencing, targeted exome sequencing, error-corrected sequencing, augmented exome sequencing, whole genome sequencing, mRNA-seq or whole transcriptome RNA-seq.

Additionally or alternatively, in some embodiments, the methods disclosed herein rely on high throughput massively parallel sequencing of a large number of diverse genes, e.g., from precancerous/cancerous or control samples. In one embodiment, the methods featured in the present technology are used in a multiplex, multi-gene assay format, e.g., assays that incorporate multiple signals from a large number of diverse genetic alterations in a large number of genes.

Additionally or alternatively, in some embodiments, a single primer or one or both primers of a primer pair comprise a specific adapter sequence (also referred to as a sequencing adapter) ligated to the 5′ end of the target specific sequence portion of the primer. This sequencing adapter is a short oligonucleotide of known sequence that can provide a priming site for both amplification and sequencing of the adjoining, unknown target nucleic acid. As such, adapters allow binding of a fragment to a flow cell for next generation sequencing. Any adapter sequence may be included in a primer used in the present technology. In certain embodiments, amplicons corresponding to specific regions of SRSF2, DNMT3A, TET2, IDH2, IDH1, TP53, SF3B1, U2AF1, or JAK2, and optionally ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, or TET1, are amplified using primers that contain an oligonucleotide sequencing adapter to produce adapter tagged amplicons.

In other embodiments, the employed primers do not contain adapter sequences and the amplicons produced are subsequently (i.e., after amplification) ligated to an oligonucleotide sequencing adapter on one or both ends of the amplicons. In some embodiments, all forward amplicons (i.e., amplicons extended from forward primers that hybridized with antisense strands of a target nucleic acid) contain the same adapter sequence. In some embodiments when double stranded sequencing is performed, all forward amplicons contain the same adapter sequence and all reverse amplicons (i.e., amplicons extended from reverse primers that hybridized with sense strands of a target segment) contain an adapter sequence that is different from the adapter sequence of the forward amplicons. In some embodiments, the adapter sequences further comprise an index sequence (also referred to as an index tag, a “barcode” or a multiplex identifier (MID)).

Additionally or alternatively, in some embodiments, the adapter sequences are P5 and/or P7 adapter sequences that are recommended for Illumina sequencers (MiSeq and HiSeq). See, e.g., Williams-Carrier et al., Plant J., 63 (1): 167-77 (2010). In some embodiments, the adapter sequences are P1, A, or Ion Xpress™ barcode adapter sequences that are recommended for Life Technologies sequencers. Other adapter sequences are known in the art. Some manufacturers recommend specific adapter sequences for use with the particular sequencing technology and machinery that they offer.

Additionally or alternatively, in some embodiments of the above methods, amplicons corresponding to specific regions of SRSF2, DNMT3A, TET2, IDH2, IDH1, TP53, SF3B1, U2AF1, or JAK2, and optionally ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, or TET1 from more than one sample are sequenced. In some embodiments, all samples are sequenced simultaneously in parallel.

In some embodiments of the above methods, amplicons corresponding to specific regions of SRSF2, DNMT3A, TET2, IDH2, IDH1, TP53, SF3B1, U2AF1, or JAK2, and optionally ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, or TET1, from at least 1, 5, 10, 20, 30 or up to 35, 40, 45, 48 or 50 different samples are amplified and sequenced using the methods described herein.

Additionally or alternatively, in some embodiments of the method, amplicons derived from a single sample may further comprise an identical index sequence that indicates the source from which the amplicon is generated, the index sequence for each sample being different from the index sequences from all other samples. As such, the use of index sequences permits multiple samples to be pooled per sequencing run and the sample source subsequently ascertained based on the index sequence. In some embodiments, the Access Array™ System (Fluidigm Corp., San Francisco, CA) or the Apollo 324 System (Wafergen Biosystems, Fremont, CA) is used to generate a barcoded (indexed) amplicon library by simultaneously amplifying the nucleic acids from the samples in one set up.

In some embodiments, indexed amplicons are generated using primers (for example, forward primers and/or reverse primers) containing the index sequence. Such indexed primers may be included during library preparation as a “barcoding” tool to identify specific amplicons as originating from a particular sample source. When adapter-ligated and/or indexed primers are employed, the adapter sequence and/or index sequence gets incorporated into the amplicon (along with the target-specific primer sequence) during amplification. Therefore, the resulting amplicons are sequencing-competent and do not require the traditional library preparation protocol. Moreover, the presence of the index tag permits the differentiation of sequences from multiple sample sources.

In some embodiments, the amplicons may be amplified with non-adapter-ligated and/or non-indexed primers and a sequencing adapter and/or an index sequence may be subsequently ligated to one or both ends of each of the resulting amplicons. In some embodiments, the amplicon library is generated using a multiplexed PCR approach.

Indexed amplicons from more than one sample source are quantified individually and then pooled prior to high throughput sequencing. As such, the use of index sequences permits multiple samples (i.e., samples from more than one sample source) to be pooled per sequencing run and the sample source subsequently ascertained based on the index sequence. “Multiplexing” is the pooling of multiple adapter-tagged and indexed libraries into a single sequencing run. When indexed primer sets are used, this capability can be exploited for comparative studies. In some embodiments, amplicon libraries from up to 48 separate sources are pooled prior to sequencing.

Following the production of an adapter tagged and, optionally indexed, amplicon library, the amplicons are sequenced using high throughput, massively parallel sequencing (i.e., next generation sequencing). Methods for performing high throughput, massively parallel sequencing are known in the art. In some embodiments of the method, the high throughput massive parallel sequencing is performed using 454TM GS FLX™ pyrosequencing, reversible dye-terminator sequencing, SOLID sequencing, ION semiconductor sequencing, Helioscope single molecule sequencing, sequencing by synthesis, sequencing by ligation, or SMRT™ sequencing. In some embodiments, high throughput massively parallel sequencing may be performed using a read depth approach.

In one aspect, the present disclosure provides a method for predicting the risk of AML in a subject prior to the onset of AML symptoms comprising detecting the presence of one or more mutations in at least one gene selected from the group consisting of SRSF2, DNMT3A, TET2, IDH2, IDH1, TP53, SF3B1, U2AF1, and JAK2 in a biological sample obtained from the subject, wherein the subject has not been diagnosed as having AML. In another aspect, the present disclosure provides a method for predicting the onset of AML symptoms in a subject that has not been diagnosed as having AML comprising detecting the presence of one or more mutations in at least one gene selected from the group consisting of SRSF2, DNMT3A, TET2, IDH2, IDH1, TP53, SF3B1, U2AF1, and JAK2 in a biological sample obtained from the subject, wherein the subject has not been diagnosed as having AML. In certain embodiments, the one or more mutations in SRSF2, U2AF1, JAK2, SF3B1, DNMT3A, TET2, IDH2, IDH1, and/or TP53 are detected using PCR (e.g., Real-time quantitative PCR (RQ-PCR), digital PCR, or reverse transcriptase PCR (RT-PCR)), Northern blots, Southern blots, microarray, dot or slot blots, in situ hybridization, electrophoresis, chromatography, mass spectroscopy, sedimentation, next-generation sequencing, Sanger sequencing, whole exome sequencing, targeted exome sequencing, error-corrected sequencing, augmented exome sequencing, whole genome sequencing, mRNA-seq or whole transcriptome RNA-seq. Additionally or alternatively, in some embodiments, the methods of the present technology further comprise detecting 2-hydroxyglutarate levels in the biological sample.

Additionally or alternatively, in some embodiments of the methods disclosed herein, the biological sample comprises one or more of genomic DNA, RNA, cDNA, cell-free DNA (cfDNA), cell-free RNA (cfRNA), and an exosome-associated nucleic acid. The biological sample may be a blood sample, a plasma sample, or a serum sample.

In any and all embodiments of the methods disclosed herein, the mutation in SRSF2, DNMT3A, TET2, IDH2, IDH1, TP53, SF3B1, U2AF1, and/or JAK2 may be a frameshift mutation, a missense mutation, a nonsense mutation, a splice site mutation, a duplication, an insertion mutation, and a deletion mutation. Additionally or alternatively, in some embodiments, the methods of the present technology further comprise detecting the presence of a mutation in one or more genes selected from the group consisting of ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, SF3B1,STAG1, STAT6, and TET1. Examples of AML symptoms include, but are not limited to, fever, fatigue, irregular heartbeat, dizziness, bone pain, frequent nosebleeds, bleeding and swollen gums, bruising on skin, loss of appetite, excessive sweating, shortness of breath, unexplained weight loss, headaches, diarrhea, menorrhagia, slurred speech, confusion, abdominal swelling, pale skin, seizures, vomiting, loss of balance, facial numbness, and blurred vision.

In any and all embodiments of the methods disclosed herein, the AML has a subtype selected from the group consisting of MO, M1, M2, M3, M4, M5, M6, M7 and M4Eo.

Examples of DNMT3A mutations include, but are not limited to p.Leu731del, p.Gly543Cys, p.Phe752Ser, p.Arg635Gly, p.Arg882Cys, p.Trp306Cys, p.Gln110AlafsTer14, p.Gly726Val, p.Phe731Leu, p.Leu905Pro, p.Arg736His, p.Gly308Arg, p.Pro904Leu, p.Arg882His, p.Ser337Leu, p.Lys766ArgfsTer13, p.Arg320Ter, p.Pro777Leu, p.Tyr533Cys, p.Arg326Cys, p.Phe755Ser, p.Arg882Ser, p. Val657Met, p. Trp313Ter, p.Arg326His, p.Tyr533Ter, p.Phe336SerfsTer9, p.Arg882Pro, p. Val328Phe, p.Arg598Ter, p. Ser770Leu, p. Thr862Ile, p.His873Pro, p.Cys557Gly, p.Arg688His, p.Gly413SerfsTer238, p. Val759TrpfsTer20, p.Phe303 SerfsTer13, p. Arg771Ter, p.Glu774Asp, p.Pro416LeufsTer235, p.Cys710Alafs Ter69, p. Phe751 SerfsTer28, p.Gly293 Arg, p.Gly728Asp, p. Trp330Ter, p.Pro743His, p. Trp860Ter, p.Leu737Arg, p.Ala254HisfsTer62, p. Val830Ter, p.Gly 706Glu, p.Gln485ArgfsTer166, p.Pro804Leu, p.Ala368Asp, p. Tyr528Asn, p.Phe732Ser, p.Arg736Cys, p. Tyr536Ter, p. Val296Met, p. Trp795Ter, p. Trp698Gly, p.Asp531Asn, p. Thr503 Asnfs Ter43, p. Pro904Gln, p. Tyr735Cys, p.Phe848Ser, p.Phe 794Leufs Ter4, p.Lys906Glu, p. Ile670HisfsTer43, p.Ile705Thr, p.Arg635Trp, p. Val895Met, p. Trp409Ter, p. Leu605AspfsTer7, p. Glu725Ter, p. Ser393 Valfs Ter14, p.Gly 796ValfsTer6, p.Cys537Arg, p.Phe414Val. p.Gln816AlafsTer42, p.Ala368Thr, p. Trp314Ter, p.Gly550Arg, p.Glu561 Ter, p.Glu733Gly, p. Tyr908Cys, p.Pro849LeufsTer4, p.Ala910Pro, p. Trp860Arg, p.Cys666TrpfsTer39, p.Arg458GlyfsTer193, p. Trp305Gly, p.Asn797ThrfsTer5, p.Ala368Val, p. Val502AspfsTer43, p. Ile780Thr, p.Met852IlefsTer29, p.Pro849Ser, p.Trp601Ter, p.Met761Val, p.Asn797Lys, p.Arg899Cys, p.Arg301Trp, and p.Arg749Gly.

Examples of IDH1 mutations include, but are not limited to p.Arg132Cys or p.Arg132Gly, and examples of IDH2 mutations include, but are not limited to p.Arg140Gln, p.Arg140His, and p.Arg140Trp. Examples of JAK2 mutations include, but are not limited to p. Val617Phe, p.Gly48Glu, and p.Glu814Gly.

Examples of SF3B1 mutations include, but are not limited to p.Arg625Leu, p.Lys790Glu, p.Gly742Asp, p.Lys700Glu, p.Ala263 Val, p.Lys666Asn, p.Ala744Val, p.His662Asp, and p.Arg625Cys. Examples of SRSF2 mutations include, but are not limited to p.Pro95His, p.Pro95Leu, p.Pro95 Arg, and p.Pro95Thr. Examples of U2AF1 mutations include, but are not limited to p.Arg156His, p.Gln157Arg, p. Tyr158dup, and p.Gln157Pro.

Examples of TET2 mutations include, but are not limited to p.Gln644Ter, p.Gln1510Ter, p.Ser167PhefsTer4, p.His1912Tyr, p.Gln180Ter, p.Gln1523Ter, p.Pro1356_Glu1357del, p.Glu1318Gly, p.Cys1263Arg, p.Glu1874Gln, p.Asn140Ser, p.Asn 140ThrfsTer8, p.Gln892Ter, p.Ile1105TyrfsTer25, p.Leu1780SerfsTer38, p. Val291GlyfsTer2, p.Asp302ValfsTer6, p.Gln706Ter, p. Val291 TrpfsTer2, p.Glu320AsnfsTer27, p.His786LeufsTer27, p.Leu1515AlafsTer62, p.Asn1489MetfsTer82, p.Arg1451GlyfsTer7, p.Met695CysfsTer5, p.Leu1151Pro, p. Val647TrpfsTer53, p.Tyr1337Ter, p.Cys1378Tyr, p.Ala727HisfsTer23, p.Glu1320ArgfsTer43, p. Trp954LeufsTer18, p. Tyr1148LeufsTer9, p.His1881Tyr, p. Val1900Gly, p.Leu1511TrpfsTer60, p.Glu537Ter, p.Gln764Ter, p.Asp1427ValfsTer22, p.Leu920SerfsTer2, p.Gln740Ter, p.Pro1594GlnfsTer37, p.Ile1873 Asn, p. Gly 1361 Asp, p.Leu500Ter, p.Gly773Ter, p.Gln321Ter, p.Gln745Ter, p.Asp1858SerfsTer10, p.Ala1158Val, p.Ser1494Ter, p.Cys1875Gly, p.Leu719Ter, p.Ala1876Val, p.Gln705Ter, p.Ser1870Leu, p.His1386Asp, p.Gln1414His, p.Asn442LysfsTer19, p.Lys664Glu, p.Arg1452Ter, p.Ser1898Pro, p.Cys1378Arg, p.Gln734Ter, p.His1904Leu, p. Thr556AsnfsTer11, p.Cys1263Tyr, p.Pro1644HisfsTer51, p.Gly641 ArgfsTer40, p.Glu1879Val, p.His1904Arg, p.Ala1512Val, p.His1904Gln, p.Met1333TyrfsTer6, p.Ile1160TyrfsTer2, p.Arg1359Ser, p.Asn258MetfsTer35, p.Lys1299Ter, p.Ala1174LysfsTer53, p.Asn442ThrfsTer5, p.Arg1516Ter, p.Gly 1275Arg, p.Ile1873Thr, p.Trp1847Ter, p.Thr229AsnfsTer25, p. Tyr1294Ter, p.Arg1712Ter, p. Tyr1902Cys, p.Leu200ThrfsTer2, p.Gly 1282Cys, p.Asp1376Gly, p. Val 1056LeufsTer10, p.Cys973LeufsTer3, p.Gln1547LeufsTer19, and p.Leu1276TrpfsTer87.

Examples of TP53 mutations include, but are not limited to p.Leu93 ArgfsTer30, p.Arg337Cys, p.Gln167Ter, p.Leu145Pro, p.Lys321Ter, p.Gln100Ter, p. Arg248Trp, p.Arg273His, p.Ala161Thr, p.Arg175Gly, p.Tyr163His, p. Tyr236His, p.Cys275Tyr, p.Ile195Ser, p.Pro128LeufsTer42, p.Tyr220Cys, p. Val272Met, p.Cys242Tyr, p.Asn29ThrfsTer15, p.Arg333 ValfsTer12, p.Met246Ile, p.Pro278His, p.Asn239Ser, p. Val 143Met, p.Arg175His, p. Thr155Asn, p. Tyr234Cys, p.Phe109SerfsTer14, and p.Cys275Phe.

In one aspect, the present disclosure provides a method of detecting the presence of at least one AML-associated mutation in a subject that does not exhibit AML symptoms and/or has not been diagnosed as having AML comprising (a) contacting a nucleic acid present in a biological sample obtained from the subject with at least one primer pair that amplifies at least one gene selected from SRSF2, DNMT3A, TET2, IDH2, IDH1, TP53, SF3B1, U2AF1, and JAK2; and (b) detecting at least one allelic variant in the nucleic acid, wherein the at least one allelic variant is
(i) a DNMT3A mutation selected from the group consisting of p.Leu731del, p.Gly543Cys, p.Phe752Ser, p.Arg635Gly, p.Arg882Cys, p. Trp306Cys, p.Gln110AlafsTer14, p.Gly726Val, p.Phe731Leu, p.Leu905Pro, p.Arg736His, p.Gly308Arg, p.Pro904Leu, p.Arg882His, p.Ser337Leu, p.Lys766ArgfsTer13, p.Arg320Ter, p.Pro777Leu, p.Tyr533Cys, p.Arg326Cys, p.Phe755Ser, p.Arg882Ser, p. Val657Met, p. Trp313Ter, p.Arg326His, p.Tyr533Ter, p.Phe336SerfsTer9, p.Arg882Pro, p. Val328Phe, p.Arg598Ter, p.Ser770Leu, p.Thr862Ile, p.His873Pro, p.Cys557Gly, p.Arg688His, p.Gly413SerfsTer238, p. Val759TrpfsTer20, p.Phe303SerfsTer13, p. Arg771Ter, p.Glu774Asp, p.Pro416LeufsTer235, p.Cys710AlafsTer69, p.Phe751 Serfs Ter28, p. Gly293 Arg, p.Gly728Asp, p. Trp330Ter, p.Pro743His, p. Trp860Ter, p.Leu737Arg, p.Ala254HisfsTer62, p. Val830Ter, p.Gly706Glu, p.Gln485ArgfsTer166, p.Pro804Leu, p.Ala368Asp, p.Tyr528Asn, p.Phe732Ser, p.Arg736Cys, p. Tyr536Ter, p. Val296Met, p. Trp795Ter, p.Trp698Gly, p.Asp531 Asn, p. Thr503 AsnfsTer43, p.Pro904Gln, p. Tyr735Cys, p.Phe848Ser, p.Phe 794Leufs Ter4, p.Lys906Glu, p.Ile670HisfsTer43, p.Ile705Thr, p.Arg635Trp, p. Val895Met, p. Trp409Ter, p.Leu605AspfsTer7, p.Glu725Ter, p. Ser393 ValfsTer14, p.Gly 796ValfsTer6, p.Cys537Arg, p.Phe414Val. p.Gln816AlafsTer42, p.Ala368Thr, p. Trp314Ter, p.Gly550Arg, p.Glu561Ter, p.Glu733Gly, p.Tyr908Cys, p.Pro849LeufsTer4, p.Ala910Pro, p. Trp860Arg, p.Cys666TrpfsTer39, p.Arg458GlyfsTer193, p. Trp305Gly, p.Asn797ThrfsTer5, p.Ala368Val, p. Val502AspfsTer43, p.Ile780Thr, p.Met852IlefsTer29, p.Pro849Ser, p.Trp601Ter, p.Met761Val, p.Asn797Lys, p.Arg899Cys, p.Arg301Trp, and p.Arg749Gly;
(ii) an IDH1 mutation selected from the group consisting of p.Arg132Cys and p.Arg132Gly;
(iii) an IDH2 mutation selected from the group consisting of p.Arg140Gln, p.Arg140His, and p.Arg140Trp;
(iv) a JAK2 mutation selected from the group consisting of p. Val617Phe, p.Gly48Glu, and p.Glu814Gly;
(v) a SF3B1 mutation selected from the group consisting of p.Arg625Leu, p.Lys790Glu, p.Gly742Asp, p.Lys700Glu, p.Ala263 Val, p.Lys666Asn, p.Ala744Val, p.His662Asp, and p.Arg625Cys;
(vi) a SRSF2 mutation selected from the group consisting of p.Pro95His, p.Pro95Leu, p.Pro95Arg, and p.Pro95Thr;
(vii) a TET2 mutation selected from the group consisting of p.Gln644Ter, p.Gln1510Ter, p.Ser167PhefsTer4, p.His1912Tyr, p.Gln180Ter, p.Gln1523Ter, p.Pro1356_Glu1357del, p.Glu1318Gly, p.Cys1263Arg, p.Glu1874Gln, p.Asn140Ser, p.Asn140ThrfsTer8, p.Gln892Ter, p.Ile1105TyrfsTer25, p.Leu1780SerfsTer38, p. Val291GlyfsTer2, p.Asp302ValfsTer6, p.Gln706Ter, p. Val291 TrpfsTer2, p.Glu320AsnfsTer27, p.His786LeufsTer27, p.Leu1515AlafsTer62, p.Asn1489MetfsTer82, p.Arg1451GlyfsTer7, p.Met695CysfsTer5, p.Leu1151Pro, p. Val647TrpfsTer53, p.Tyr1337Ter, p.Cys1378Tyr, p.Ala727HisfsTer23, p.Glu1320ArgfsTer43, p. Trp954LeufsTer18, p. Tyr1148LeufsTer9, p.His1881Tyr, p. Val1900Gly, p.Leu1511TrpfsTer60, p.Glu537Ter, p.Gln764Ter, p.Asp1427ValfsTer22, p.Leu920SerfsTer2, p.Gln740Ter, p.Pro1594GlnfsTer37, p.Ile1873Asn, p.Gly 1361Asp, p.Leu500Ter, p.Gly773Ter, p.Gln321Ter, p.Gln745Ter, p.Asp1858SerfsTer10, p.Ala1158Val, p.Ser1494Ter, p.Cys1875Gly, p.Leu719Ter, p.Ala1876Val, p.Gln705Ter, p.Ser1870Leu, p.His1386Asp, p.Gln1414His, p.Asn442LysfsTer19, p.Lys664Glu, p.Arg1452Ter, p.Ser1898Pro, p.Cys1378Arg, p.Gln734Ter, p.His1904Leu, p. Thr556AsnfsTer11, p.Cys1263Tyr, p.Pro1644HisfsTer51, p.Gly641 ArgfsTer40, p.Glu1879Val, p.His1904Arg, p.Ala1512Val, p.His1904Gln, p.Met1333TyrfsTer6, p.Ile1160TyrfsTer2, p.Arg1359Ser, p.Asn258MetfsTer35, p.Lys1299Ter, p.Ala1174LysfsTer53, p.Asn442ThrfsTer5, p.Arg1516Ter, p.Gly 1275Arg, p. Ile1873Thr, p.Trp1847Ter, p.Thr229AsnfsTer25, p.Tyr1294Ter, p.Arg1712Ter, p. Tyr1902Cys, p.Leu200ThrfsTer2, p.Gly 1282Cys, p.Asp1376Gly, p. Val 1056LeufsTer10, p.Cys973LeufsTer3, p.Gln1547LeufsTer19, and p.Leu1276TrpfsTer87;
(viii) a TP53 mutation selected from the group consisting of p.Leu93 ArgfsTer30, p.Arg337Cys, p.Gln167Ter, p.Leu145Pro, p.Lys321Ter, p.Gln100Ter, p.Arg248Trp, p.Arg273His, p.Ala161Thr, p.Arg175Gly, p. Tyr163His, p. Tyr236His, p.Cys275Tyr, p.Ile195Ser, p.Pro128LeufsTer42, p.Tyr220Cys, p. Val272Met, p.Cys242Tyr, p. Asn29ThrfsTer15, p. Arg333 ValfsTer12, p.Met246Ile, p. Pro278His, p.Asn239Ser, p. Val143Met, p.Arg175His, p. Thr155Asn, p.Tyr234Cys, p.Phe109SerfsTer14, and p.Cys275Phe; or
(ix) an U2AF1 mutation selected from the group consisting of p.Arg156His, p.Gln157Arg, p. Tyr158dup, and p.Gln157Pro, wherein detecting the at least one allelic variant is indicative of the presence of at least one AML-associated mutation in the subject.

In another aspect, the present disclosure provides a method of detecting the presence of at least one AML-associated mutation in a subject that does not exhibit AML symptoms and/or has not been diagnosed as having AML comprising (a) contacting a nucleic acid present in a biological sample obtained from the subject with one or more probes that hybridize to at least one gene selected from SRSF2, DNMT3A, TET2, IDH2, IDH1, TP53, SF3B1, U2AF1, and JAK2, wherein the one or more probes can recognize and discriminate allelic variants of SRSF2, DNMT3A, TET2, IDH2, IDH1, TP53, SF3B1, U2AF1, and JAK2; and (b) detecting at least one allelic variant in the nucleic acid, wherein the at least one allelic variant is
(i) a DNMT3A mutation selected from the group consisting of p.Leu731del, p.Gly543Cys, p.Phe752Ser, p.Arg635Gly, p.Arg882Cys, p. Trp306Cys, p.Gln110AlafsTer14, p.Gly 726Val, p.Phe731Leu, p.Leu905Pro, p.Arg736His, p.Gly308Arg, p.Pro904Leu, p.Arg882His, p.Ser337Leu, p.Lys766ArgfsTer13, p.Arg320Ter, p.Pro777Leu, p.Tyr533Cys, p.Arg326Cys, p.Phe755Ser, p.Arg882Ser, p. Val657Met, p. Trp313Ter, p.Arg326His, p. Tyr533Ter, p.Phe336SerfsTer9, p.Arg882Pro, p. Val328Phe, p.Arg598Ter, p.Ser770Leu, p.Thr862Ile, p.His873Pro, p.Cys557Gly, p.Arg688His, p.Gly413SerfsTer238, p. Val759TrpfsTer20, p.Phe303SerfsTer13, p.Arg771Ter, p.Glu774Asp, p.Pro416LeufsTer235, p.Cys710AlafsTer69, p. Phe751 SerfsTer28, p. Gly293 Arg, p.Gly728Asp, p. Trp330Ter, p.Pro743His, p. Trp860Ter, p.Leu737Arg, p.Ala254HisfsTer62, p. Val830Ter, p.Gly 706Glu, p.Gln485ArgfsTer166, p.Pro804Leu, p.Ala368Asp, p.Tyr528Asn, p.Phe732Ser, p.Arg736Cys, p. Tyr536Ter, p. Val296Met, p.Trp795Ter, p. Trp698Gly, p.Asp531Asn, p. Thr503 AsnfsTer43, p.Pro904Gln, p. Tyr735Cys, p.Phe848Ser, p.Phe 794Leufs Ter4, p.Lys906Glu, p.Ile670HisfsTer43, p.Ile705Thr, p.Arg635Trp, p. Val895Met, p.Trp409Ter, p.Leu605AspfsTer7, p.Glu725Ter, p. Ser393 ValfsTer14, p.Gly796ValfsTer6, p.Cys537Arg, p.Phe414Val. p.Gln816AlafsTer42, p.Ala368Thr, p.Trp3 14Ter, p.Gly550Arg, p. Glu561Ter, p.Glu733Gly, p. Tyr908Cys, p.Pro849LeufsTer4, p.Ala910Pro, p.Trp860Arg, p.Cys666TrpfsTer39, p.Arg458GlyfsTer193, p. Trp305Gly, p.Asn797ThrfsTer5, p.Ala368Val, p. Val502AspfsTer43, p. Ile780Thr, p.Met852IlefsTer29, p.Pro849Ser, p.Trp601Ter, p.Met761Val, p.Asn797Lys, p.Arg899Cys, p.Arg301Trp, and p.Arg749Gly;
(ii) an IDH1 mutation selected from the group consisting of p.Arg132Cys and p.Arg132Gly;
(iii) an IDH2 mutation selected from the group consisting of p.Arg140Gln, p.Arg140His, and p.Arg140Trp;
(iv) a JAK2 mutation selected from the group consisting of p. Val617Phe, p.Gly48Glu, and p.Glu814Gly;
(v) a SF3B1 mutation selected from the group consisting of p.Arg625Leu, p.Lys790Glu, p.Gly742Asp, p.Lys700Glu, p.Ala263 Val, p.Lys666Asn, p.Ala744Val, p.His662Asp, and p.Arg625Cys;
(vi) a SRSF2 mutation selected from the group consisting of p.Pro95His, p.Pro95Leu, p.Pro95Arg, and p.Pro95Thr;
(vii) a TET2 mutation selected from the group consisting of p.Gln644Ter, p.Gln1510Ter, p.Ser167PhefsTer4, p.His1912Tyr, p.Gln180Ter, p.Gln1523Ter, p.Pro1356_Glu1357del, p.Glu1318Gly, p.Cys1263Arg, p.Glu1874Gln, p.Asn140Ser, p.Asn 140ThrfsTer8, p.Gln892Ter, p.Ile1105TyrfsTer25, p.Leu1780SerfsTer38, p. Val291GlyfsTer2, p.Asp302ValfsTer6, p.Gln706Ter, p. Val291 TrpfsTer2, p.Glu320AsnfsTer27, p.His786LeufsTer27, p.Leu1515AlafsTer62, p.Asn1489MetfsTer82, p.Arg1451GlyfsTer7, p.Met695CysfsTer5, p.Leu1151Pro, p. Val647TrpfsTer53, p. Tyr1337Ter, p.Cys1378Tyr, p.Ala727HisfsTer23, p.Glu1320ArgfsTer43, p. Trp954LeufsTer18, p. Tyr1148LeufsTer9, p.His1881Tyr, p. Val1900Gly, p.Leu1511TrpfsTer60, p.Glu537Ter, p.Gln764Ter, p.Asp1427ValfsTer22, p.Leu920SerfsTer2, p.Gln740Ter, p.Pro1594GInfsTer37, p.Ile1873Asn, p.Gly 1361Asp, p.Leu500Ter, p.Gly773Ter, p.Gln321Ter, p.Gln745Ter, p.Asp1858SerfsTer10, p.Ala1158Val, p.Ser1494Ter, p.Cys1875Gly, p.Leu719Ter, p.Ala1876Val, p.Gln705Ter, p.Ser1870Leu, p.His1386Asp, p.Gln1414His, p.Asn442LysfsTer19, p.Lys664Glu, p.Arg1452Ter, p.Ser1898Pro, p.Cys1378Arg, p.Gln734Ter, p.His1904Leu, p.Thr556AsnfsTer11, p.Cys1263Tyr, p.Pro1644HisfsTer51, p.Gly641 ArgfsTer40, p.Glu1879Val, p.His1904Arg, p.Ala1512Val, p.His1904Gln, p.Met1333TyrfsTer6, p.Ile1160TyrfsTer2, p.Arg1359Ser, p.Asn258MetfsTer35, p.Lys1299Ter, p.Ala1174LysfsTer53, p.Asn442ThrfsTer5, p.Arg1516Ter, p.Gly 1275Arg, p. Ile1873Thr, p.Trp1847Ter, p.Thr229AsnfsTer25, p. Tyr1294Ter, p.Arg1712Ter, p. Tyr1902Cys, p.Leu200ThrfsTer2, p.Glyl282Cys, p.Asp1376Gly, p. Val 1056LeufsTer10, p.Cys973LeufsTer3, p.Gln1547LeufsTer19, and p.Leu1276TrpfsTer87;
(viii) a TP53 mutation selected from the group consisting of p.Leu93 ArgfsTer30, p.Arg337Cys, p.Gln167Ter, p.Leu145Pro, p.Lys321Ter, p.Gln100Ter, p.Arg248Trp, p.Arg273His, p.Ala161Thr, p.Arg175Gly, p.Tyr163His, p. Tyr236His, p.Cys275Tyr, p.Ile195Ser, p.Pro128LeufsTer42, p.Tyr220Cys, p. Val272Met, p.Cys242Tyr, p.Asn29ThrfsTer15, p.Arg333 ValfsTer12, p.Met246Ile, p. Pro278His, p.Asn239Ser, p. Val 143Met, p.Arg175His, p. Thr155Asn, p. Tyr234Cys, p.Phe109SerfsTer14, and p.Cys275Phe; or
(ix) an U2AF1 mutation selected from the group consisting of p.Arg156His, p.Gln157Arg, p.Tyr158dup, and p.Gln157Pro, wherein detecting the at least one allelic variant is indicative of the presence of at least one AML-associated mutation in the subject.

### Methods of Treatment of the Present Technology

Disclosed herein are methods for determining whether a patient at risk for AML (i.e., prior to the onset of AML symptoms), will benefit from or is predicted to be responsive to a particular therapy for AML.

In one aspect, the present disclosure provides a method for selecting a subject at risk for AML for treatment with an AML therapy comprising (a) detecting the presence of one or more mutations in SRSF2, U2AF1, and JAK2 in a biological sample obtained from the subject; and (b) selecting the subject for treatment with an AML therapy, wherein the subject does not exhibit AML symptoms, and wherein the AML therapy comprises one or more of chemotherapeutic agents, FLT3 inhibitors, IDH inhibitors, Gemtuzumab ozogamicin, BCL-2 inhibitors, Hedgehog pathway inhibitors, All-trans-retinoic acid, and Arsenic trioxide.

In one aspect, the present disclosure provides a method for selecting a subject at risk for AML for treatment with an AML therapy comprising (a) detecting the presence of one or more mutations in SRSF2, DNMT3A, TET2, IDH2, IDH1, TP53, SF3B1, U2AF1, and JAK2 in a biological sample obtained from the subject; and (b) selecting the subject for treatment with an AML therapy, wherein the subject does not exhibit AML symptoms, and wherein the AML therapy comprises one or more of chemotherapeutic agents, FLT3 inhibitors, IDH inhibitors, Gemtuzumab ozogamicin, BCL-2 inhibitors, Hedgehog pathway inhibitors, All-trans-retinoic acid, and Arsenic trioxide.

In another aspect, the present disclosure provides a method for preventing or delaying the onset of AML symptoms in a subject at risk for AML comprising administering an effective amount of an AML therapy to the subject, wherein the subject harbors a mutation in one or more genes selected from the group consisting of SRSF2, DNMT3A, TET2, IDH2, IDH1, TP53, SF3B1, U2AF1, and JAK2, and wherein the AML therapy comprises one or more of chemotherapeutic agents, FLT3 inhibitors, IDH inhibitors, Gemtuzumab ozogamicin, BCL-2 inhibitors, Hedgehog pathway inhibitors, All-trans-retinoic acid, and Arsenic trioxide. Examples of AML symptoms include, but are not limited to, fever, fatigue, irregular heartbeat, dizziness, bone pain, frequent nosebleeds, bleeding and swollen gums, bruising on skin, loss of appetite, excessive sweating, shortness of breath, unexplained weight loss, headaches, diarrhea, menorrhagia, slurred speech, confusion, abdominal swelling, pale skin, seizures, vomiting, loss of balance, facial numbness, and blurred vision.

Examples of DNMT3A mutations include, but are not limited to p.Leu731del, p.Gly543Cys, p.Phe752Ser, p.Arg635Gly, p.Arg882Cys, p.Trp306Cys, p.Gln110AlafsTer14, p.Gly726Val, p.Phe731Leu, p. Leu905Pro, p.Arg736His, p.Gly308Arg, p.Pro904Leu, p.Arg882His, p.Ser337Leu, p.Lys766ArgfsTer13, p.Arg320Ter, p.Pro777Leu, p.Tyr533Cys, p.Arg326Cys, p.Phe755Ser, p.Arg882Ser, p. Val657Met, p. Trp313Ter, p.Arg326His, p.Tyr533Ter, p.Phe336SerfsTer9, p.Arg882Pro, p. Val328Phe, p.Arg598Ter, p. Ser770Leu, p.Thr862Ile, p.His873Pro, p.Cys557Gly, p.Arg688His, p.Gly413SerfsTer238, p. Val759TrpfsTer20, p.Phe303 SerfsTer13, p.Arg771Ter, p.Glu774Asp, p.Pro416LeufsTer235, p.Cys710AlafsTer69, p.Phe751 SerfsTer28, p. Gly293 Arg, p.Gly728Asp, p. Trp330Ter, p.Pro743His, p. Trp860Ter, p.Leu737Arg, p.Ala254HisfsTer62, p. Val830Ter, p.Gly706Glu, p.Gln485ArgfsTer166, p.Pro804Leu, p.Ala368Asp, p. Tyr528Asn, p.Phe732Ser, p.Arg736Cys, p. Tyr536Ter, p. Val296Met, p. Trp795Ter, p. Trp698Gly, p.Asp531Asn, p. Thr503 AsnfsTer43, p.Pro904Gln, p. Tyr735Cys, p.Phe848Ser, p.Phe 794Leufs Ter4, p.Lys906Glu, p.Ile670HisfsTer43, p.Ile705Thr, p.Arg635Trp, p. Val895Met, p. Trp409Ter, p. Leu605AspfsTer7, p.Glu725Ter, p. Ser393 Valfs Ter14, p.Gly796ValfsTer6, p.Cys537Arg, p.Phe414Val. p.Gln816AlafsTer42, p.Ala368Thr, p. Trp314Ter, p.Gly550Arg, p.Glu561Ter, p. Glu733Gly, p.Tyr908Cys, p.Pro849LeufsTer4, p.Ala910Pro, p. Trp860Arg, p.Cys666TrpfsTer39, p.Arg458GlyfsTer193, p. Trp305Gly, p. Asn797ThrfsTer5, p.Ala368Val, p. Val502AspfsTer43, p. Ile780Thr, p.Met852IlefsTer29, p.Pro849Ser, p.Trp601Ter, p.Met761Val, p.Asn797Lys, p.Arg899Cys, p.Arg301Trp, and p.Arg749Gly.

Examples of IDH1 mutations include, but are not limited to p.Arg132Cys or p.Arg132Gly, and examples of IDH2 mutations include, but are not limited to p.Arg140Gln, p.Arg140His, and p.Arg140Trp. Examples of JAK2 mutations include, but are not limited to p. Val617Phe, p.Gly48Glu, and p.Glu814Gly.

Examples of SF3B1 mutations include, but are not limited to p.Arg625Leu, p.Lys790Glu, p.Gly742Asp, p.Lys700Glu, p.Ala263 Val, p.Lys666Asn, p.Ala744Val, p.His662Asp, and p.Arg625Cys. Examples of SRSF2 mutations include, but are not limited to p.Pro95His, p.Pro95Leu, p.Pro95Arg, and p.Pro95Thr. Examples of U2AF1 mutations include, but are not limited to p.Arg156His, p.Gln157Arg, p.Tyr158dup, and p.Gln157Pro.

Examples of TET2 mutations include, but are not limited to p.Gln644Ter, p.Gln1510Ter, p.Ser167PhefsTer4, p.His1912Tyr, p.Gln180Ter, p.Gln1523Ter, p.Pro1356_Glu1357del, p.Glu1318Gly, p.Cys1263Arg, p.Glu1874Gln, p.Asn140Ser, p.Asn140ThrfsTer8, p.Gln892Ter, p.Ile1105TyrfsTer25, p.Leu1780SerfsTer38, p. Val291GlyfsTer2, p.Asp302ValfsTer6, p.Gln706Ter, p. Val291 TrpfsTer2, p.Glu320AsnfsTer27, p.His786LeufsTer27, p.Leu1515AlafsTer62, p.Asn1489MetfsTer82, p.Arg1451GlyfsTer7, p.Met695CysfsTer5, p.Leu1151Pro, p. Val647TrpfsTer53, p. Tyr1337Ter, p.Cys1378Tyr, p.Ala727HisfsTer23, p.Glu1320ArgfsTer43, p. Trp954LeufsTer18, p. Tyr1148LeufsTer9, p.His1881Tyr, p. Val1900Gly, p.Leu1511TrpfsTer60, p.Glu537Ter, p.Gln764Ter, p.Asp1427ValfsTer22, p.Leu920SerfsTer2, p.Gln740Ter, p.Pro1594GInfsTer37, p.Ile1873Asn, p.Gly 1361Asp, p.Leu500Ter, p.Gly773Ter, p.Gln321Ter, p.Gln745Ter, p.Asp1858SerfsTer10, p.Ala1158Val, p.Ser1494Ter, p.Cys1875Gly, p.Leu719Ter, p.Ala1876Val, p.Gln705Ter, p.Ser1870Leu, p.His1386Asp, p.Gln1414His, p.Asn442LysfsTer19, p.Lys664Glu, p.Arg1452Ter, p.Ser1898Pro, p.Cys1378Arg, p.Gln734Ter, p.His1904Leu, p. Thr556AsnfsTer11, p.Cys1263Tyr, p.Pro1644HisfsTer51, p.Gly641 ArgfsTer40, p.Glu1879Val, p.His1904Arg, p.Ala1512Val, p.His1904Gln, p.Met1333TyrfsTer6, p.Ile1160TyrfsTer2, p.Arg1359Ser, p.Asn258MetfsTer35, p.Lys1299Ter, p.Ala1174LysfsTer53, p.Asn442ThrfsTer5, p.Arg1516Ter, p.Gly 1275Arg, p. Ile1873Thr, p.Trp1847Ter, p.Thr229AsnfsTer25, p. Tyr1294Ter, p.Arg1712Ter, p. Tyr1902Cys, p.Leu200ThrfsTer2, p.Gly 1282Cys, p.Asp1376Gly, p. Val 1056LeufsTer10, p.Cys973LeufsTer3, p.Gln1547LeufsTer19, and p.Leu1276TrpfsTer87.

Examples of TP53 mutations include, but are not limited to p.Leu93 ArgfsTer30, p.Arg337Cys, p.Gln167Ter, p.Leu145Pro, p.Lys321Ter, p.Gln100Ter, p.Arg248Trp, p.Arg273His, p.Ala161Thr, p.Arg175Gly, p.Tyr163His, p. Tyr236His, p.Cys275Tyr, p.Ile195Ser, p.Pro128LeufsTer42, p.Tyr220Cys, p. Val272Met, p.Cys242Tyr, p.Asn29ThrfsTer15, p.Arg333 ValfsTer12, p.Met246Ile, p.Pro278His, p.Asn239Ser, p. Val 143Met, p.Arg175His, p.Thr155Asn, p.Tyr234Cys, p.Phe109SerfsTer14, and p.Cys275Phe.

In any of the above embodiments of the methods disclosed herein, the subject harbors an additional mutation in one or more genes selected from the group consisting of ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, SF3B1, STAG1, STAT6, and TET1.

Additionally or alternatively, in some embodiments, the chemotherapeutic agents comprise one or more of cytarabine, an anthracycline drug (e.g., daunorubicin (daunomycin), doxorubicin, or idarubicin), cladribine, fludarabine, mitoxantrone, etoposide (VP-16), 6-thioguanine (6-TG), hydroxyurea, corticosteroid drugs (such as prednisone or dexamethasone), Methotrexate (MTX), 6-mercaptopurine (6-MP), azacitidine, and decitabine (Dacogen).

Examples of FLT3 inhibitors include, but are not limited to, midostaurin, lestaurtinib, sunitinib, sorafenib, gilteritinib, quizartinib, crenolanib, tandutinib, ponatinib, PLX3397, KW-2449, and ASP2215. Examples of IDH inhibitors include, but are not limited to AG-881 (Vorasidenib), ivosidenib, enasidenib, BAY-1436032, AGI-5198, IDH305, AGI-6780, FT-2102, HMS-101, MRK-A, and GSK321.

Examples of BCL-2 inhibitors include, but are not limited to ABT-199 (venetoclax), HA14-1, obatoclax (GX-15-070), ABT-737, GDC-0199, and ABT-263 (navitoclax). Examples of Hedgehog pathway inhibitors include, but are not limited to glasdegib, vismodegib, sonidegib, GANT-58 and GANT-61, Arsenic Trioxide, RU-SKI 43, and 5E1 monoclonal antibody.

### Kits

The present disclosure also provides kits for detecting alterations in nucleic acid sequences corresponding to SRSF2, DNMT3A, TET2, IDH2, IDH1, TP53, SF3B1, U2AF1, and JAK2, and optionally ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1

Kits of the present technology comprise one or more primer pairs that selectively hybridize and are useful in amplifying one or more of the genes selected from the group consisting of SRSF2, DNMT3A, TET2, IDH2, IDH1, TP53, SF3B1, U2AF1, JAK2, ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1.

In some embodiments, the kits of the present technology comprise a single primer pair, probe, or bait that hybridizes to an exon or an intron of a single gene selected from the group consisting of SRSF2, DNMT3A, TET2, IDH2, IDH1, TP53, SF3B1, U2AF1, JAK2, ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1.

In other embodiments, the kits of the present technology comprise multiple primer pairs, probes, or baits that hybridize to one or more exons and/or introns of a single gene selected from the group consisting of SRSF2, DNMT3A, TET2, IDH2, IDH1, TP53, SF3B1, U2AF1, JAK2, ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1.

In certain embodiments, the kits of the present technology comprise multiple primer pairs comprising a single primer pair that specifically hybridizes to an exon or intron of a single gene for each of SRSF2, DNMT3A, TET2, IDH2, IDH1, TP53, SF3B1, U2AF1, JAK2, ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1. Additionally or alternatively, in certain embodiments, the kits of the present technology comprise multiple probes or baits comprising a single probe or bait that specifically hybridizes to an exon or intron of a single gene for each of SRSF2, DNMT3A, TET2, IDH2, IDH1, TP53, SF3B1, U2AF1, JAK2, ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1.

In certain embodiments, the kits of the present technology comprise multiple primer pairs comprising more than one primer pair that hybridizes to one or more exons and/or introns for each of SRSF2, DNMT3A, TET2, IDH2, IDH1, TP53, SF3B1, U2AF1, JAK2, ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1. Additionally or alternatively, in some embodiments, the kits of the present technology comprise multiple probes or baits comprising more than one probe or bait that hybridizes to one or more exons and/or introns for each of SRSF2, DNMT3A, TET2, IDH2, IDH1, TP53, SF3B1, U2AF1, JAK2, ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1.

Thus, it is contemplated herein that the kits of the present technology can comprise primer pairs, baits, or probes that recognize and specifically hybridize to one or more exons and/or introns of one or more genes selected from the group consisting of SRSF2, DNMT3A, TET2, IDH2, IDH1, TP53, SF3B1, U2AF1, JAK2, ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1.

Alternatively, the kit can comprise primer pairs, probes, and/or baits that will detect one or more mutations in at least one gene selected from the group consisting of SRSF2, DNMT3A, TET2, IDH2, IDH1, TP53, SF3B1, U2AF1, and JAK2. Examples of DNMT3A mutations include, but are not limited to p. Leu731del, p.Gly543Cys, p.Phe752Ser, p.Arg635Gly, p.Arg882Cys, p. Trp306Cys, p.Gln110AlafsTer14, p.Gly726Val, p.Phe731Leu, p.Leu905Pro, p.Arg736His, p.Gly308Arg, p.Pro904Leu, p.Arg882His, p.Ser337Leu, p.Lys766ArgfsTer13, p.Arg320Ter, p.Pro777Leu, p.Tyr533Cys, p.Arg326Cys, p.Phe755Ser, p.Arg882Ser, p. Val657Met, p. Trp313Ter, p.Arg326His, p.Tyr533Ter, p.Phe336SerfsTer9, p.Arg882Pro, p. Val328Phe, p.Arg598Ter, p.Ser770Leu, p.Thr862Ile, p.His873Pro, p.Cys557Gly, p.Arg688His, p.Gly413SerfsTer238, p. Val759TrpfsTer20, p.Phe303 SerfsTer13, p.Arg771Ter, p.Glu774Asp, p.Pro416LeufsTer235, p.Cys710Alafs Ter69, p.Phe 751 SerfsTer28, p.Gly293 Arg, p.Gly 728Asp, p. Trp330Ter, p.Pro743His, p. Trp860Ter, p.Leu737Arg, p.Ala254HisfsTer62, p. Val830Ter, p.Gly706Glu, p.Gln485ArgfsTer166, p.Pro804Leu, p.Ala368Asp, p.Tyr528Asn, p.Phe732Ser, p.Arg736Cys, p. Tyr536Ter, p. Val296Met, p. Trp795Ter, p. Trp698Gly, p.Asp531 Asn, p. Thr503 AsnfsTer43, p.Pro904Gln, p.Tyr735Cys, p.Phe848Ser, p.Phe794Leufs Ter4, p.Lys906Glu, p.Ile670HisfsTer43, p.Ile705Thr, p.Arg635Trp, p. Val895Met, p. Trp409Ter, p.Leu605 AspfsTer7, p. Glu725Ter, p. Ser393 Valfs Ter14, p. Gly 796Valfs Ter6, p.Cys537Arg, p.Phe414Val. p.Gln816AlafsTer42, p.Ala368Thr, p. Trp314Ter, p.Gly550Arg, p.Glu561Ter, p.Glu733Gly, p. Tyr908Cys, p. Pro849LeufsTer4, p.Ala910Pro, p. Trp860Arg, p.Cys666TrpfsTer39, p.Arg458GlyfsTer193, p. Trp305Gly, p.Asn797ThrfsTer5, p.Ala368Val, p. Val502AspfsTer43, p.Ile780Thr, p.Met852IlefsTer29, p.Pro849Ser, p. Trp601Ter, p.Met761Val, p.Asn797Lys, p.Arg899Cys, p.Arg301Trp, and p.Arg749Gly.

Examples of IDH1 mutations include, but are not limited to p.Arg132Cys or p.Arg132Gly, and examples of IDH2 mutations include, but are not limited to p.Arg140Gln, p.Arg140His, and p.Arg140Trp. Examples of JAK2 mutations include, but are not limited to p. Val617Phe, p.Gly48Glu, and p.Glu814Gly.

Examples of SF3B1 mutations include, but are not limited to p.Arg625Leu, p.Lys790Glu, p.Gly742Asp, p.Lys700Glu, p.Ala263 Val, p.Lys666Asn, p.Ala744Val, p.His662Asp, and p.Arg625Cys. Examples of SRSF2 mutations include, but are not limited to p.Pro95His, p.Pro95Leu, p.Pro95Arg, and p.Pro95Thr. Examples of U2AF1 mutations include, but are not limited to p.Arg156His, p.Gln157Arg, p.Tyr158dup, and p.Gln157Pro.

Examples of TET2 mutations include, but are not limited to p.Gln644Ter, p.Gln1510Ter, p.Ser167PhefsTer4, p.His1912Tyr, p.Gln180Ter, p.Gln1523Ter, p.Pro1356_Glu1357del, p.Glu1318Gly, p.Cys1263Arg, p.Glu1874Gln, p.Asn140Ser, p.Asn 140ThrfsTer8, p.Gln892Ter, p.Ile1 105TyrfsTer25, p.Leu1780SerfsTer38, p. Val291GlyfsTer2, p.Asp302ValfsTer6, p.Gln706Ter, p. Val291 TrpfsTer2, p.Glu320AsnfsTer27, p.His786LeufsTer27, p.Leu1515AlafsTer62, p.Asn1489MetfsTer82, p.Arg1451GlyfsTer7, p.Met695CysfsTer5, p.Leu1151Pro, p. Val647TrpfsTer53, p.Tyr1337Ter, p.Cys1378Tyr, p.Ala727HisfsTer23, p.Glu1320ArgfsTer43, p. Trp954LeufsTer18, p. Tyr1148LeufsTer9, p.His1881Tyr, p. Val 1900Gly, p.Leu1511TrpfsTer60, p.Glu537Ter, p.Gln764Ter, p.Asp1427ValfsTer22, p.Leu920SerfsTer2, p.Gln740Ter, p.Pro1594GInfsTer37, p.Ile1873Asn, p.Gly 1361Asp, p.Leu500Ter, p.Gly773Ter, p.Gln321Ter, p.Gln745Ter, p.Asp1858SerfsTer10, p.Ala1158Val, p.Ser1494Ter, p.Cys1875Gly, p.Leu719Ter, p.Ala1876Val, p.Gln705Ter, p.Ser1870Leu, p.His1386Asp, p.Gln1414His, p.Asn442LysfsTer19, p.Lys664Glu, p.Arg1452Ter, p.Ser1898Pro, p.Cys1378Arg, p.Gln734Ter, p.His1904Leu, p. Thr556AsnfsTer11, p.Cys1263Tyr, p.Pro1644HisfsTer51, p.Gly641 ArgfsTer40, p.Glu1879Val, p.His1904Arg, p.Ala1512Val, p.His1904Gln, p.Met1333TyrfsTer6, p.Ile1160TyrfsTer2, p.Arg1359Ser, p.Asn258MetfsTer35, p.Lys1299Ter, p.Ala1174LysfsTer53, p.Asn442ThrfsTer5, p.Arg1516Ter, p.Gly 1275Arg, p. Ile1873Thr, p.Trp1847Ter, p.Thr229AsnfsTer25, p.Tyr1294Ter, p.Arg1712Ter, p. Tyr1902Cys, p.Leu200ThrfsTer2, p.Glyl282Cys, p.Asp1376Gly, p. Val 1056LeufsTer10, p.Cys973LeufsTer3, p.Gln1547LeufsTer19, and p.Leu1276TrpfsTer87.

Examples of TP53 mutations include, but are not limited to p.Leu93 ArgfsTer30, p.Arg337Cys, p.Gln167Ter, p.Leu145Pro, p.Lys321Ter, p.Gln100Ter, p.Arg248Trp, p.Arg273His, p.Ala161Thr, p.Arg175Gly, p. Tyr163His, p. Tyr236His, p.Cys275Tyr, p.Ile195Ser, p.Pro128LeufsTer42, p.Tyr220Cys, p. Val272Met, p.Cys242Tyr, p.Asn29ThrfsTer15, p.Arg333 ValfsTer12, p.Met246Ile, p.Pro278His, p.Asn239Ser, p. Val 143Met, p.Arg175His, p. Thr155Asn, p. Tyr234Cys, p.Phe109SerfsTer14, and p.Cys275Phe.

In some embodiments, the kits further comprise buffers, enzymes having polymerase activity, enzymes having polymerase activity and lacking 5′ →3′ exonuclease activity or both 5′→3′ and 3′→5′ exonuclease activity, enzyme cofactors such as magnesium or manganese, salts, chain extension nucleotides such as deoxynucleoside triphosphates (dNTPs), modified dNTPs, nuclease-resistant dNTPs or labeled dNTPs, necessary to carry out an assay or reaction, such as amplification and/or detection of alterations in target nucleic acid sequences corresponding to SRSF2, DNMT3A, TET2, IDH2, IDH1, TP53, SF3B1, U2AF1, and JAK2, and optionally ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1.

In one embodiment, the kits of the present technology further comprise a positive control nucleic acid sequence and a negative control nucleic acid sequence to ensure the integrity of the assay during experimental runs. A kit may further contain a means for comparing the levels and/or activity of one or more of SRSF2, DNMT3A, TET2, IDH2, IDH1, TP53, SF3B1, U2AF1, and JAK2, and optionally ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1 in a precancerous/cancerous sample with a reference nucleic acid sample (e.g., a non-tumor sample). The kit may also comprise instructions for use, software for automated analysis, containers, packages such as packaging intended for commercial sale and the like.

The kits of the present technology can also include other necessary reagents to perform any of the NGS techniques disclosed herein. For example, the kit may further comprise one or more of: adapter sequences, barcode sequences, reaction tubes, ligases, ligase buffers, wash buffers and/or reagents, hybridization buffers and/or reagents, labeling buffers and/or reagents, and detection means. The buffers and/or reagents are usually optimized for the particular amplification/detection technique for which the kit is intended. Protocols for using these buffers and reagents for performing different steps of the procedure may also be included in the kit.

The kits of the present technology may include components that are used to prepare nucleic acids from a test sample (e.g., blood, serum, plasma) for the subsequent amplification and/or detection of alterations in target nucleic acid sequences corresponding to SRSF2, DNMT3A, TET2, IDH2, IDH1, TP53, SF3B1, U2AF1, and JAK2, and optionally ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, STAG1, STAT6, and TET1. Such sample preparation components can be used to produce nucleic acid extracts from tissue samples. The test samples used in the above-described methods will vary based on factors such as the assay format, nature of the detection method, and the specific tissues, cells or extracts used as the test sample to be assayed. Methods of extracting nucleic acids from samples are well known in the art and can be readily adapted to obtain a sample that is compatible with the system utilized. Automated sample preparation systems for extracting nucleic acids from a test sample are commercially available, e.g., Roche Molecular Systems' COBAS AmpliPrep System, Qiagen's BioRobot 9600, and Applied Biosystems' PRISM™ 6700 sample preparation system.

### EXAMPLES

The present technology is further illustrated by the following Examples, which should not be construed as limiting in any way.

### Example 1: Experimental Materials and Methods

De-identified samples and data were obtained from the Women's Health Initiative prospective clinical trial (WHI, Controlled Clinical Trials 19, 61-109 (1998); Anderson et al., Annals of Epidemiology 13, S5-17 (2003)) and collection of such was performed with the requisite approval. Informed consent from participants was obtained at the onset of the WHI trial conducted by participating centers. Detailed clinical data regarding medical history, medications, and complete blood counts were available at baseline assessment. New diagnoses were updated in follow up, with central confirmation of all new cancer diagnoses.

Identification of cases: In the WHI cohort, 212 study participants eventually developed pathologically confirmed AML. Of these, baseline peripheral blood (PB) DNA was available and passed quality control in 189 participants; these were identified as cases to be included in the final analyses. Additional follow up samples for 132 cases were available at 1 year and/or 3 years after baseline, all prior to the diagnosis of AML. Exclusion criteria for cases included known diagnosis of any myeloid disorder, including AML, prior to WHI baseline evaluation

Controls: Age-matched controls (N=212) were selected from participants who were confirmed not to have a diagnosis of AML while being followed on the WHI study. Exclusion criteria included concurrent or history of prior myeloid disorder, including AML, at WHI baseline. Controls were matched to cases by age at baseline, history of non-myeloid cancers at baseline and type and timing of any cancers that occurred in cases after WHI baseline, but before the diagnosis of AML as well as timing of blood sample collection. Matching was done in a time forward manner to ensure that each control had as much control time as its matched case (Bergstralh, E. J., Kosanke, J. L. & Jacobsen, S. J., Epidemiology 7, 331-332 (1996)). Of the 212 controls, PB was available and passed quality standard in 183 controls at WHI baseline and these were included in the analyses. Additional follow up samples for 128 controls were available at 1 year and/or 3 years after baseline.

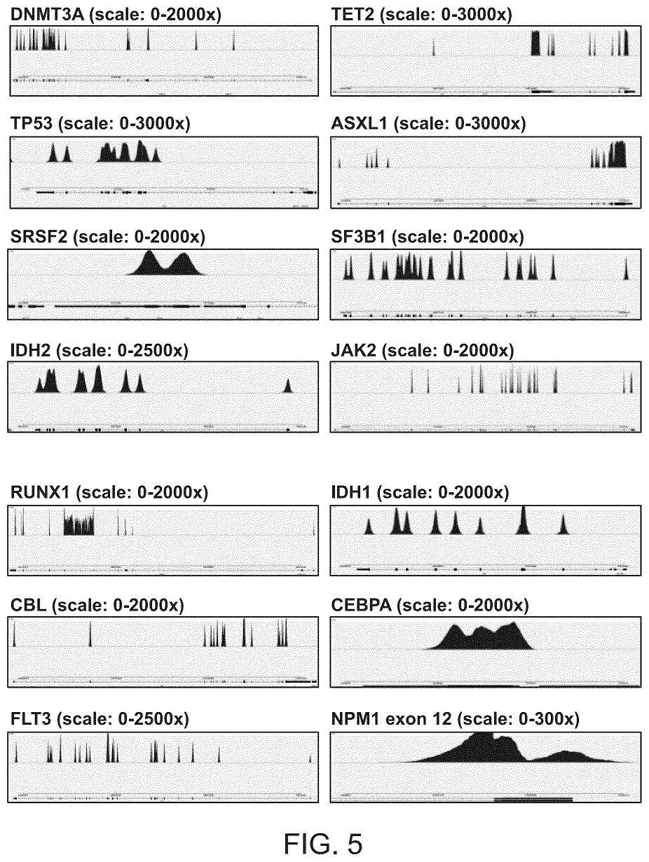

Genomic DNA was provided by WHI in a blinded manner, in which case-control status and clinical covariates were revealed only after variant calling was completed. Library generation and amplification were performed using a low error rate Hi-Fi DNA polymerase according to the Kapa HyperPrep protocol (Kapa Biosystems). Dual sample indexing, rather than single indexing, of libraries was performed to minimize signal spread errors arising from misidentification of multiplexed samples. Targeted sequencing of genes recurrently mutated in hematological malignancies was performed using custom capture probes (Nimblegen) to a median coverage of 2000× for both AML cases and controls (FIG. 5). Reads were aligned to the 1000 genomes phase 2 human reference genome+decoy contigs (hs37d5) using BWA MEM (Li, H. ar Xiv preprint ar Xiv 00, 3-3, (2013)). Variants were detected using VarDictJava (Lai, Z. et al., Nucleic Acids Res 44, e108 (2016)) using a 1% VAF cutoff and filtered for artifacts as described (Li, H. Bioinformatics 30, 2843-2851 (2014)).

Study population: The WHI enrolled more than 160,000 women in one or more of four clinical trials (CT group) or an observational study cohort (OS group) in 40 U.S. clinical centers from Oct. 1, 1993 through Dec. 31, 1998 with data collection updated through September 2012 and an average follow up of 10.8 years (SD 3.3 years). The participants in the CT group were followed at baseline and years 1, 3 and 6, with samples collected at WHI baseline, year 1 and 3 during these follow up visits. The participants in the OS group were followed at baseline and year 3 with samples collected similarly during these visits.

Targeted Exome Sequencing. Genomic DNA was provided by WHI in a blinded manner, in which case-control status and clinical covariates were revealed only after variant calling was completed. Library generation and amplification were performed using a low error rate Hi-Fi DNA polymerase according to the Kapa HyperPrep protocol (Kapa Biosystems). Dual sample indexing, rather than single indexing, of libraries was performed to minimize signal spread errors arising from misidentification of multiplexed samples. Targeted sequencing using a panel of 67 recurrently mutated genes in hematological malignancies was performed using a custom capture probes (Nimblegen) to a median coverage of 2000× for both AML cases and controls (FIG. 5). Variant analysis was performed following rigorous quality control and filtration of low quality sequence information. To identify somatic variants, filtration based on population allele frequency data was applied so as to enrich somatic variants that are not likely inherited. To this end, variants were classified as probable somatic if exhibiting a dbSNP v142 or ExAC adjusted population allele frequency <=0.25% or a median VAF in the cohort <40%. Only mutations present at >1% VAF were evaluated for association with AML development and time-to-AML.

Statistical Analysis. Baseline characteristics of AML cases and matched controls were compared with the use of the two-sample t-test for continuous variables and the Fisher's exact test for categorical variables. Among the 189 cases, participants with baseline precursor mutations were compared to participants without precursor mutations with regard to demographic characteristics and baseline hematological characteristics (i.e., WBC count and differential counts, hemoglobin value and platelet count). The relationship between specific precursor mutations and AML development were estimated by exact odds ratios (OR) and adjusted ORs were obtained from penalized-likelihood logistic regression. OR and adjusted ORs are presented with their associated 95% confidence interval (95% CI). Multivariable penalized-likelihood logistic regression analysis was performed to assess the independent effect of demographic and prognostic factors of interest on precursor mutation status. Collinearity between predictors in the models was evaluated prior to the formulation of the final multivariable models. Time to development of AML was estimated with a Kaplan-Meier estimator. Differences between groups based on mutational status were evaluated with a log-rank test. Significant differences in variant allele fraction were determined in serial sampling using Fisher's Exact Test based on the count of supporting alternate and reference reads for each sample at a mutated site. All p-values were two-sided with statistical significance set a priori at the 0.05 level. Ninety-five percent confidence intervals (95% C.I.) were calculated to assess the precision of the obtained estimates. All statistical analyses were performed with the use of R software (version 3.4.0).

Statistical tools. All statistical analysis was performed using the R statistical programming language. Multivariate odds ratios and p-values were computed using Firth's Bias-Reduced Logistic Regression implemented in the logistf package. Plots were produced using ggplot2 package. Data summarization and reshaping were performed using plyr, dplyr and reshape 2 packages.

Next generation sequencing. Following targeted enrichment according to Nimblegen protocols, libraries were sequenced on the Illumina HiSeq 4000 using dual-indexed sample adapters (Integrated DNA Technologies). To reduce errors arising from misalignment, reads were trimmed of contaminating adapter sequences and low-quality bases using Trimmomatic v0.32 (trimmed when median Illumina base quality score <20 over 6 nt sliding window). To further improve sequence quality, overlapping paired end reads were merged into a single long consensus read using AdapterRemoval v2 when at least 12 bp overlap was present. The remaining high-quality reads were mapped against the 1000 genomes phase 2 human reference genome+decoy contigs (hs37d5) using BWA MEM10. Duplicate marking was performed using SamBlaster and MarkDupsByStartEnd. Single nucleotide variants (SNVs) and insertions/deletions (indels) were detected using VarDictJava in single sample mode with indel realignment. Marked duplicates were excluded. Copy number variations (CNV) were detected using CNVkit. Annotation of variants and their functional impact was performed using Variant Effect Predictor (VEP) and snpEff. Indel representations in both the call set and annotation data were left-aligned and harmonized using vt analysis toolkit to maximize concordance between variants and annotations.

The resulting call set was filtered in accordance with guidance regarding artifact removal as described. Variants were classified as probable artifacts if any of the following conditions were met: occurs within a low complexity region subject to high alignment error defined in the hs37d5 reference genome according to the mdust-LC algorithm; exhibits strand bias; present within or immediately flanked by repetitive or low entropy regions (10 bp window); <4 supporting reads per strand except for NPM1 exon 12 (Ensembl transcript ENST00000517671.5) where any supporting reads indicative of insertions longer than 4 nucleotides were considered; mean BWA MEM mapping quality of supporting reads <45; mean Illumina base quality of variant-supporting bases <30; exhibits read position bias toward the 5′ or 3′ end of reads; <100× unique depth of high quality coverage at site of mutation; coverage depth x VAF <8; exhibits high recurrence suggestive of artifact except for mutations known to be present >=10 times in COSMIC v74 or at least once in the TCGA AML study when recurrence is defined by an identical mutation occurring in >20% of samples evaluated or the position is mutated in >40% of evaluated samples or identical mutation is present in >1 sequenced instance of NA12878 or the identical position is mutated in >2 sequenced instances of NA12878. Finally, mutations classified by VEP were considered when categorized as missense, stop gain, splice acceptor, splice donor, frameshift insertion, frameshift deletion, in-frame insertion, and in-frame deletion mutations.

Analyzed genes. Coding exons and flanking DNA+/−5 bp were evaluated for mutations in 67 genes including ASXL1, ASXL2, ATRX, BCOR, BCORL1, BRAF, CALR, CARD11, CBL, CBLB, CBLC, CD70, CD79B, CDKN2A, CEBPA, CREBBP, CSF3R, CUX1, DIS3, DNMT3A, EPPK1, ETV6, EZH2, FBXW7, FLT1, FLT3, GATA1, GATA2, GNAS, HRAS, IDH1, IDH2, IKZF1, JAK1, JAK2, JAK3, KDM6A, KIT, KRAS, MPL, MYD88, NOTCH1, NPM1, NRAS, PAX5, PDGFRA, PHF6, PTEN, PTPN11, RAD21, RUNX1, SETBP1, SF3B1, SMCIA, SMC3, SRSF2, STAG1, STAG2, STAT6, TET1, TET2, TNF, TNFRSF14, TP53, U2AF1, WT1, ZRSR2. Depth of coverage statistics were determined using the Picard tools package.

Pathogenicity of variants. Detected variants were classified as “probable pathogenic” if any of the following conditions were met: Known hotspot in genes of known pathogenic significance in myeloid malignancies including DNMT3A, IDH1, IDH2, SRSF2, SF3B1, TP53, JAK2, FLT3, NRAS, KRAS, KIT, MPL, CBL; Disruptive mutations that produce frameshifts, duplications, stop codons, or affect splice acceptor or donor sites in genes where such alterations are known to be of pathogenic impact in myeloid malignancies including DNMT3A, TET2, TP53, NPM1 exon 12, FLT3 exon 14, RUNX1, ASXL1 exon 12, (′ALR exon 9, CEBPA, ATRX; Mutations demonstrating ≥10 instances in COSMIC v74 or previously identified in the AML TCGA study; or SNVs classified as pathogenic using the Mendelian Clinically Applicable Pathogenicity (M-CAP) score classifier.

Visualization and assessment of mutation spectrum, co-mutation spectrum, and clonal evolution. Variants and clinical annotations were visualized as an OncoPrint using the ComplexHeatmap package. Chord diagrams indicating co-mutational patterns of myeloid malignancy genes were producing using the circlize v0.4.3 package. Mutual exclusivity of and co-occurrence of mutated genes were determined using the maftools v1.4.25 R package.

Determination of nucleotide composition and context of mutations. Analysis of transitions, transversions, and relative proportion of mutation types was performed in R/BioConductor using the maftools package. Relative frequencies of transitions and transversions within trinucleotide and transcribed strand context was performed using the R/BioConductor MutationalPatterns package using transcript strand notations derived from known transcript data present in the BioConductor
TxDb.Hsapiens.ucsc.hg19.knowngene database with hg19 coordinates converted to hs37d5.

Functional domain analysis. To determine the presence of mutations within known functional domains, individual mutations were mapped to Pfam functional domains based on the gene and protein-level HGVS description of each alteration (e.g., DNMT3A p.Arg882Cys) with the maftools package. Quantification and plotting of most frequently altered Pfam functional domains and the number of involved genes was performed using maftools package. 3-D special clusters were identified using mutation3D. 3-D molecule visualizations were generated using PyMol 2.0 (Schrödinger, LLC; New York, NY).

### Example 2: Clonal Mutations in Peripheral Blood are Evident Years Prior to the Diagnosis of AMI

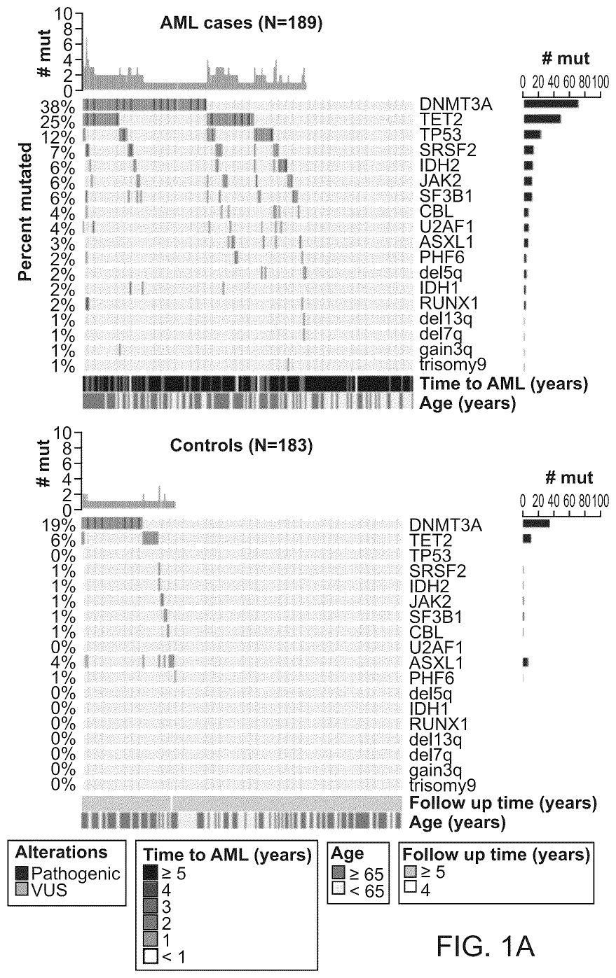

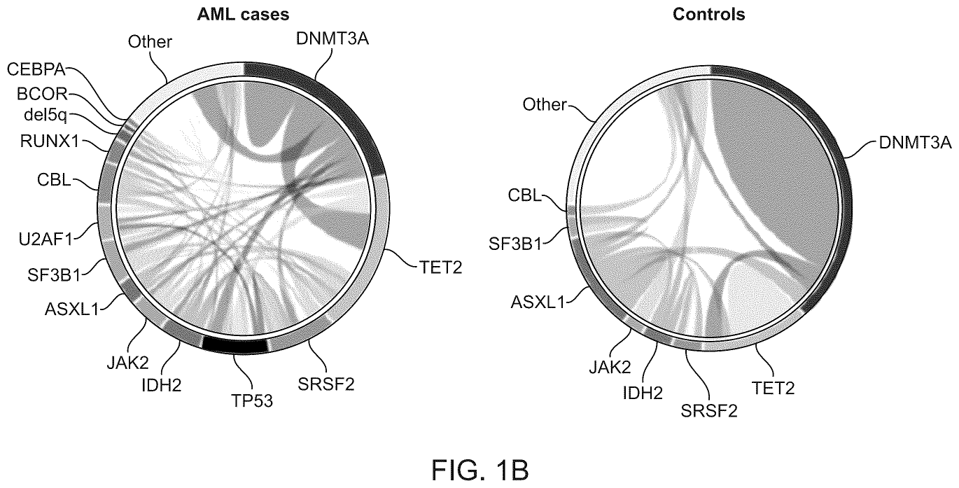

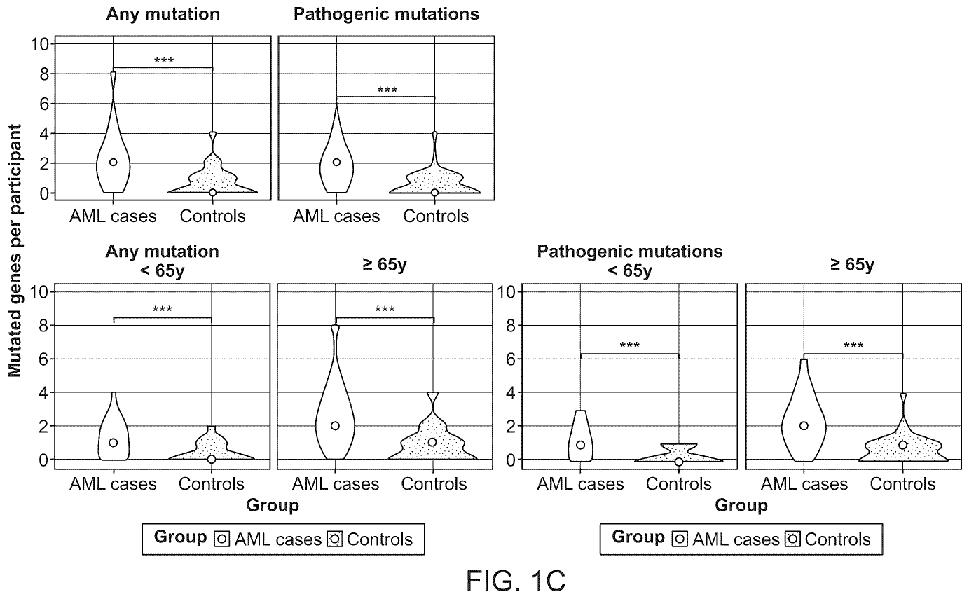

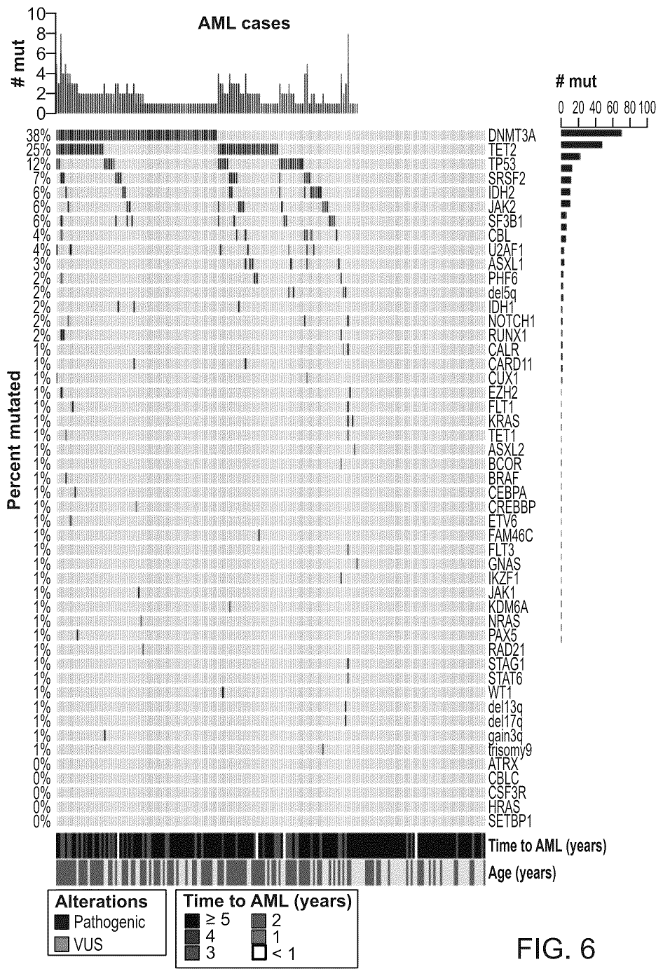

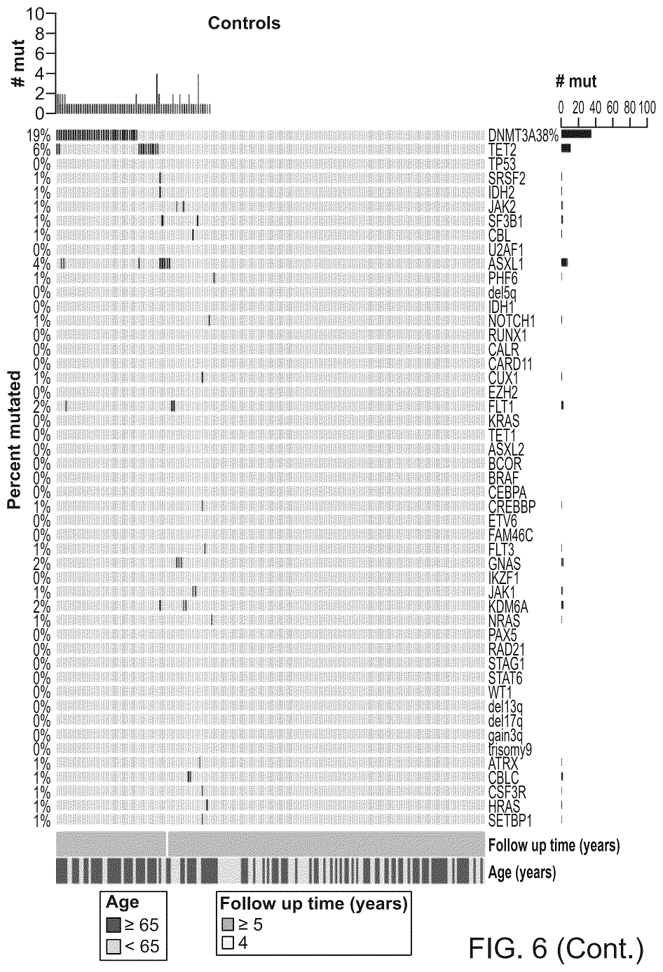

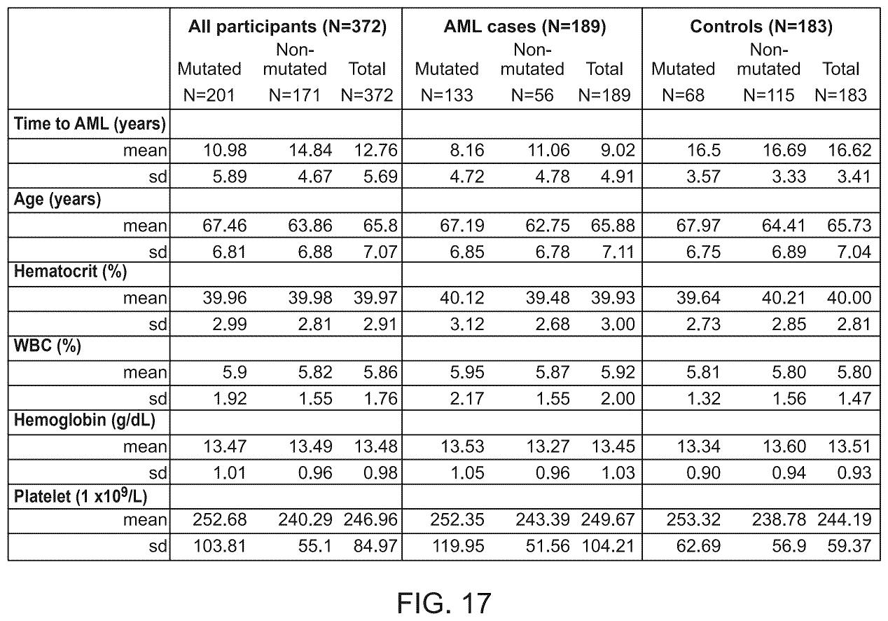

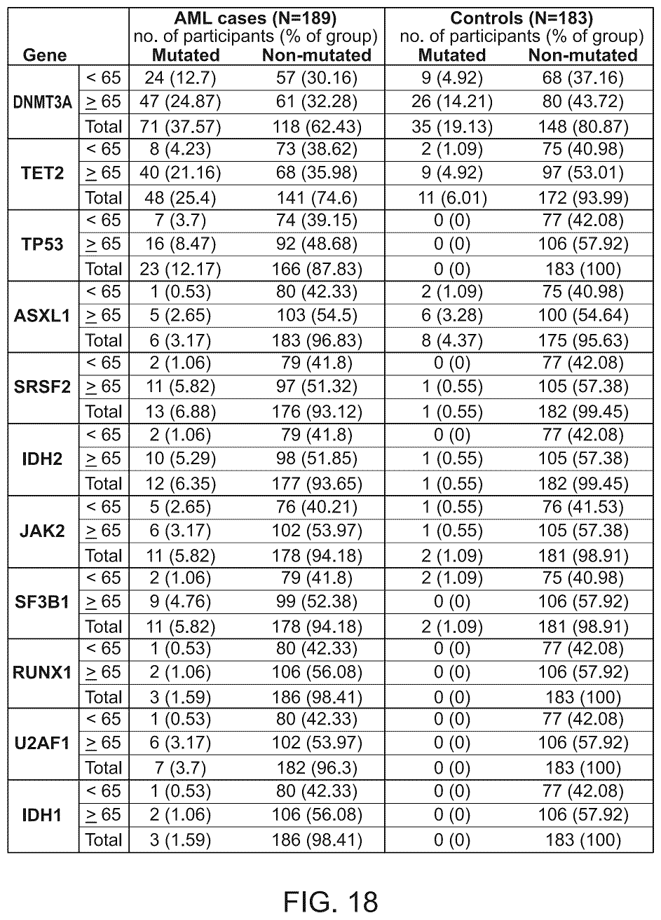

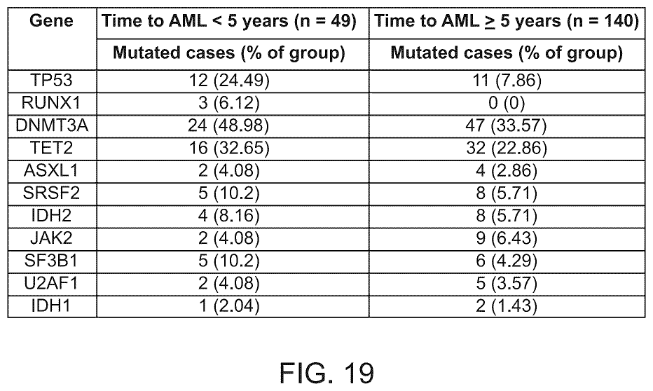

At a median of 9.8 years prior to the diagnosis of AML, cases were more likely to harbor mutations than controls (OR 4.0, 95% C.I. 2.5-6.3, P<0.001). The most common mutations identified above 1% variant allele fraction (VAF) included DNMT3A (37.6% cases vs. 19.1% controls), TET2 (25.4% cases vs. 6.0% controls), TP53 (12.2% cases vs. 0% controls), SRSF2 (6.8% cases vs. 0.5% controls), IDH2 (6.3% cases vs. 0.5% controls), SF3B1 (5.8% cases vs. 0.5% controls), JAK2 (5.8% cases vs. 1.1% controls), and ASXL1 (3.2% cases vs. 4.4% controls; FIG. 1A and FIG. 6). In aggregate, spliceosome mutations as a group (SF3B1, SRSF2, and U2AF1) were identified in 14.2% of cases (N=27/189) vs. 1.6% of controls (N=3/183). Similarly, IDH mutations as a group (IDH1 and IDH2) were identified in 7.9% of cases (N=15/189) vs. 0.5% of controls (N=1/183). There was no association between the presence of any mutation and abnormal hemoglobin, white blood cell (WBC) count and/or platelet level (FIG. 17).

AML cases overall demonstrated greater clonal complexity than controls, with 55.6% of the AML cases harboring co-mutations, compared to 17.6% of controls (OR 5.2, 95% C.I. 2.5-11.7, P<0.001). As shown in FIG. 1B, the most common co-mutations present in AML cases were DNMT3A with TET2, DNMT3A with TP53, DNMT3A with SRSF2, TET2 with SRSF2, and IDH2 with SRSF2. Among controls, mutations were generally present individually, and the only common co-mutation was DNMT3A with TET2. Overall, AML cases harbored a median of 1 mutated gene (median 1, range 0-8) while controls harbored median of 0 mutated genes (median 0, range 0-4) (P<0.001). Older individuals (≥65 years) harbored more mutated genes in both AML cases and controls. The same held true for mutations categorized as pathogenic versus variant of unknown significance (VUS) (FIG. 1C).

### Example 3: The Presence of Somatic Mutations at Baseline Assessment was Associated with Significantly Increased Odds of Developing AML

Table 1A shows the number and frequency of mutations in AML cases vs. controls overall and for participants younger than 65 years vs. ≥65 years.

Having a mutation at baseline assessment was associated with statistically increased odds of developing AML (OR 4.0, 95% C.I. 2.5-6.3, P<0.001) (Table 1A). This finding was independent of age: OR 3.5 (95% C.I. 1.8-7.3) for age <65 years; OR 5.1 (95% C.I. 2.7-10.2) for age ≥65 years. Overall, 70.4% (N=133) of cases and only 37.2% of controls (N=68) were found to have mutations. Mutations were found in 55.6% of cases and 26.0% of controls <65 years old, and in 81.5% of cases and 45.3% of controls age ≥65 years.

TABLE 1A

| | AML cases (N = 189) | | Controls (N = 183) | | Odds Ratio | |
|---|---|---|---|---|---|---|
| Age | # Mutated (%) | # Non-mutated (%) | # Mutated (%) | # Non-mutated (%) | OR (95% CI) | p value |
| <65 | 45 (23.81) | 36 (19.05) | 20 (10.93) | 57 (31.15) | 3.54 (1.8-7.3) | <0.001 |
| ≥65 | 88 (46.56) | 20 (10.58) | 48 (26.23) | 58 (31.69) | 5.12 (2.66-10.19) | <0.001 |
| Total | 133 (70.37) | 56 (29.63) | 68 (37.16) | 115 (62.84) | 4.00 (2.54-6.34) | <0.001 |

Among the recurrently mutated genes, some mutations demonstrated increased specificity and penetrance for the development of AML. All participants with a TP53 (N-23/23) or RUNX1 (N=3/3) mutation developed AML. Also, all participants with an IDH1 or IDH2 mutation eventually developed AML except one control, which was lost to follow-up at the end of the study. Multivariable analysis was performed to evaluate potential associations between individual mutated genes and the development of AML, adjusting for confounders including co-mutated genes and age. Table 1B shows a Forest plot indicating odds ratio of mutations in each gene occurring in the AML cases vs. controls. Genes or gene categories significantly associated with AML include TP53, IDH, spliceosome, TET2, and DNMT3A.

TABLE 1B
Odds ratio: AML cases vs. controls

| Gene | N | Odds Ratio (95% CI) | p value |
|---|---|---|---|
| TP53 | 23 | 54.2 (2.89, 1017.7) | <0.001 |
| IDH | 16 | 8.42 (1.37, 51.88) | 0.004 |
| RUNX1 | 3 | 6.31 (0.08, 504.07) | 0.233 |
| Spliceosome | 30 | 5.57 (1.5, 20.63) | 0.004 |
| TET2 | 59 | 5.44 (2.5, 11.87) | <0.001 |
| JAK2 | 13 | 3.84 (0.79, 18.72) | 0.065 |
| DNMT3A | 106 | 2.73 (1.61, 4.63) | <0.001 |
| ASXL1 | 14 | 0.42 (0.12, 1.56) | 0.178 |
| Age | 372 | 1.04 (1.01, 1.08) | 0.011 |

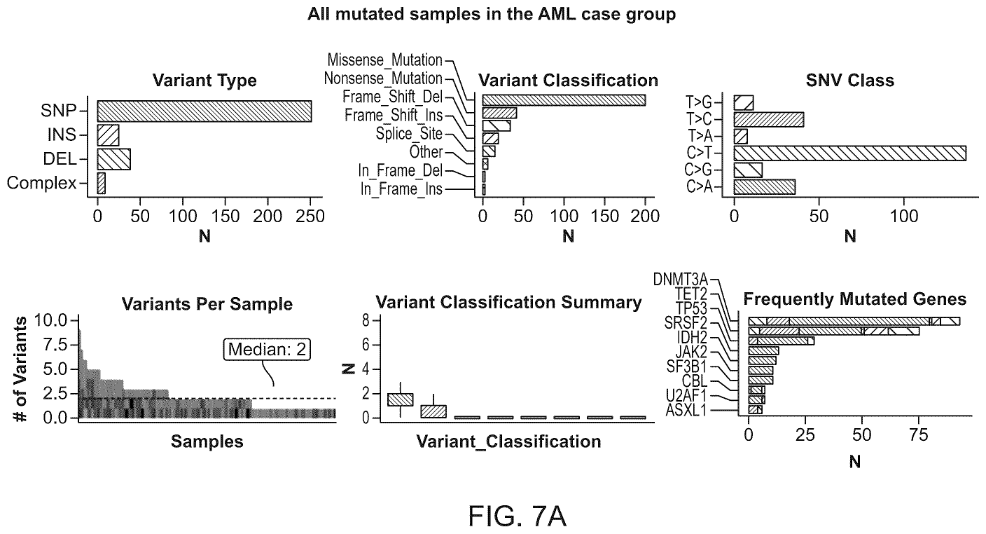

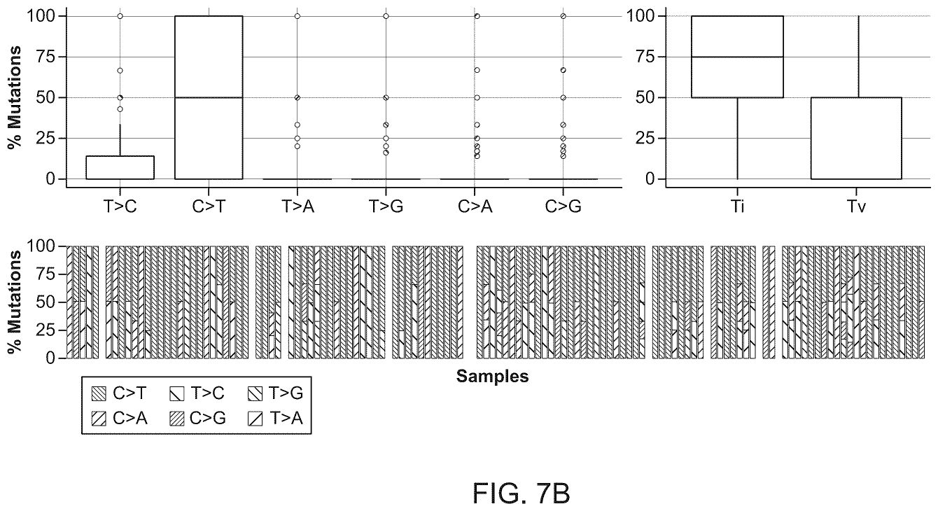

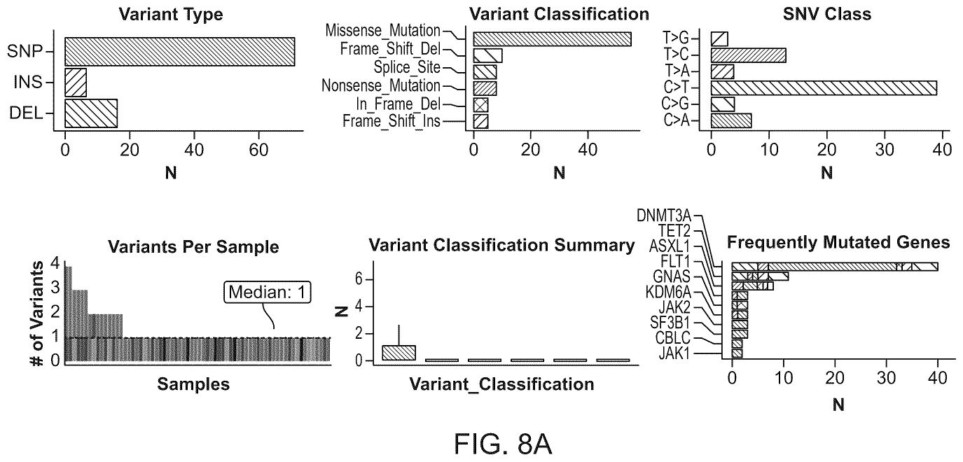

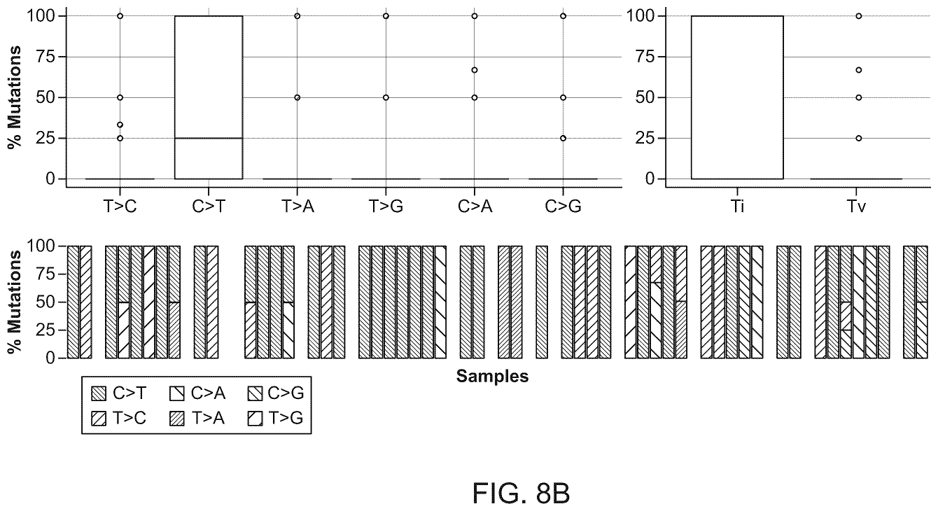

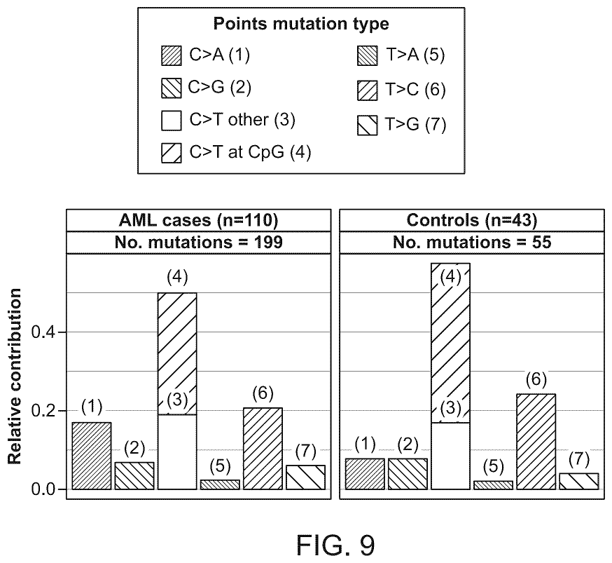

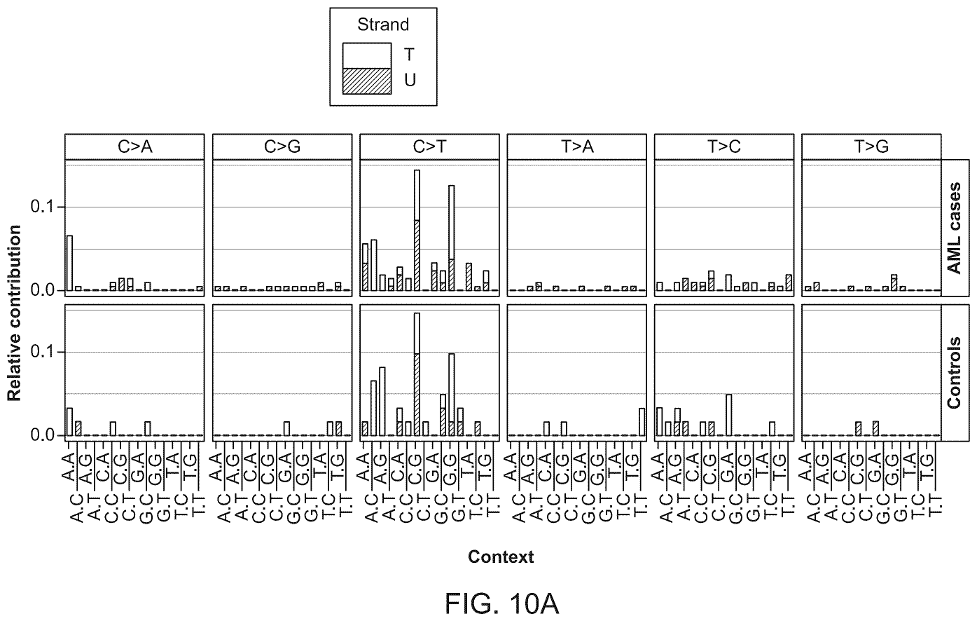

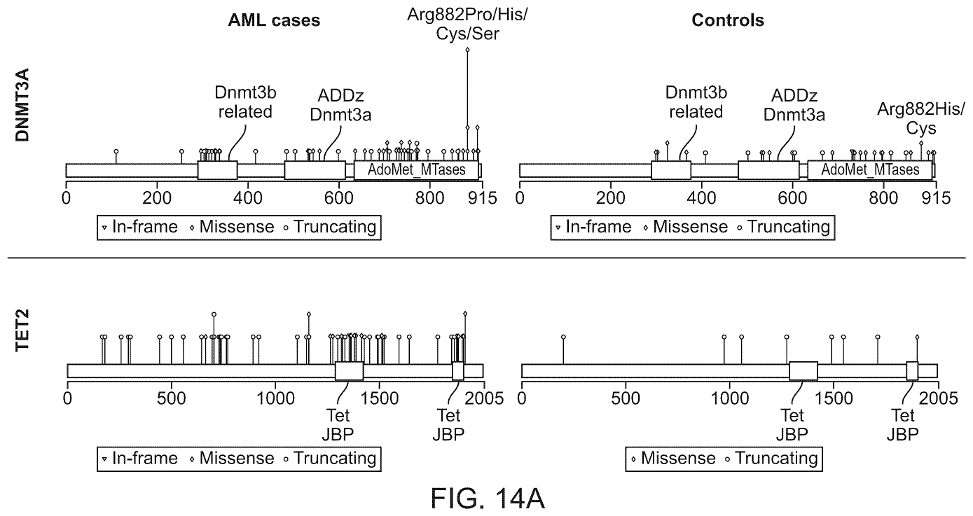

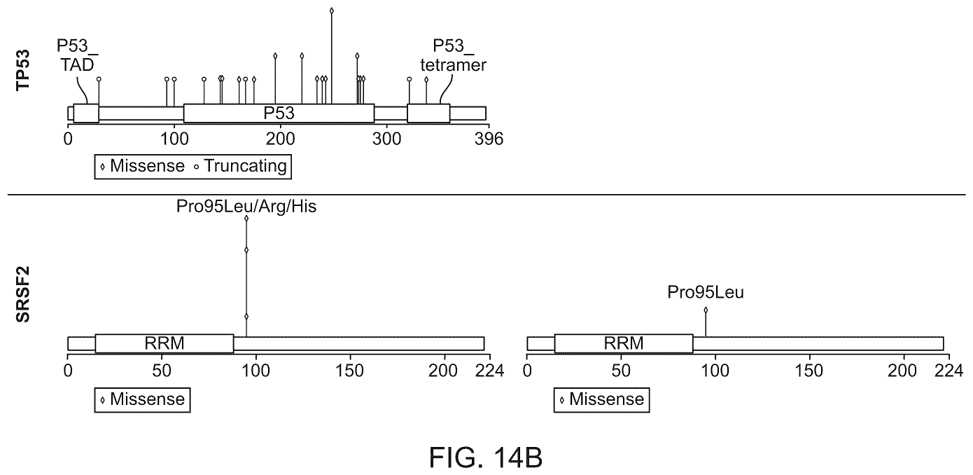

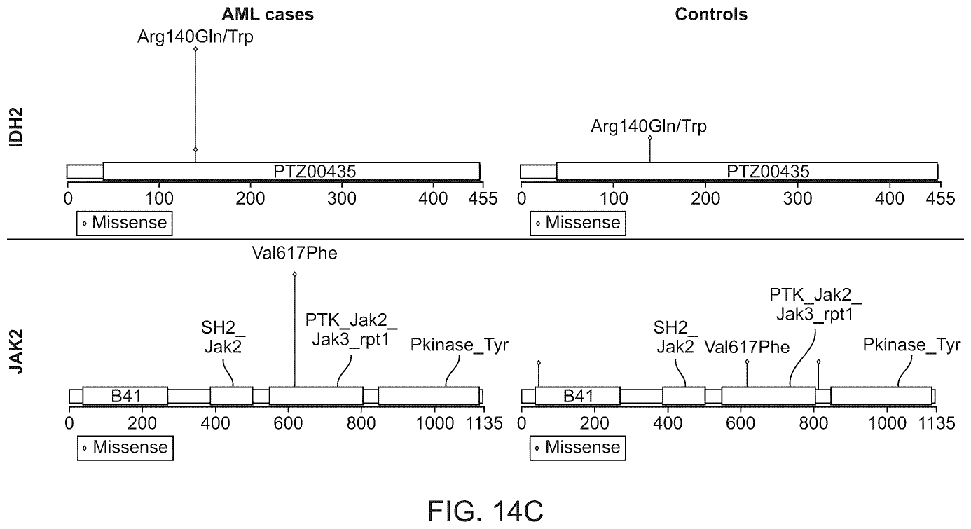

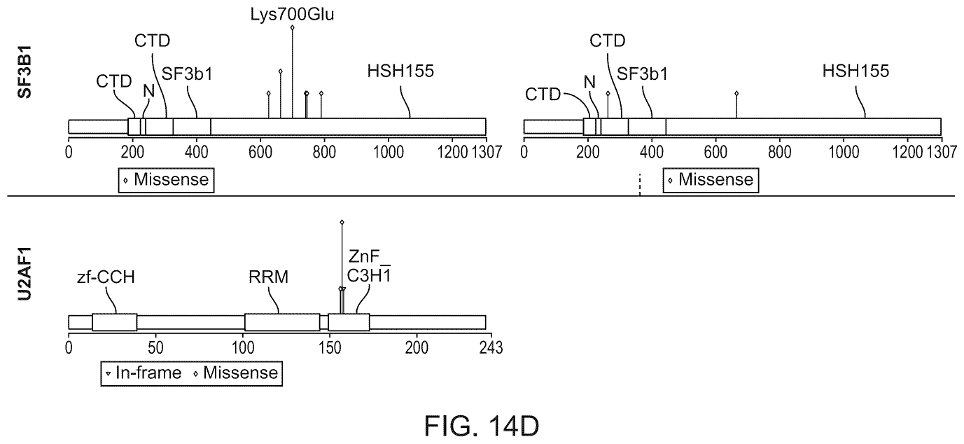

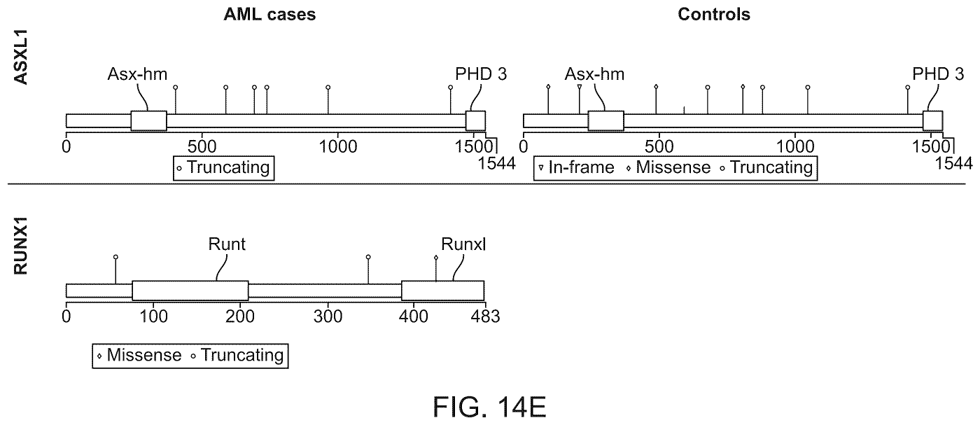

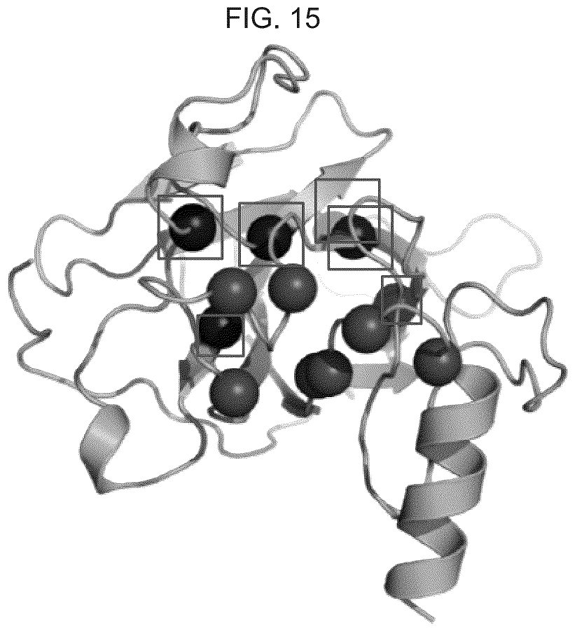

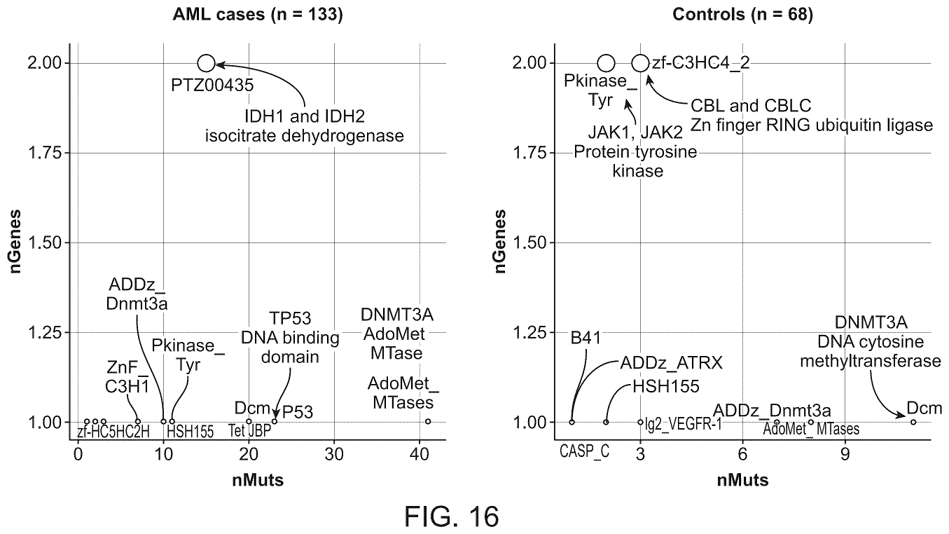

This analysis found that TP53 (OR 54.2, 95% C.I. 2.9-1017.7), IDH (including IDH1 and IDH2) (OR 8.4, 95% C.I. 1.4-51.9), spliceosome genes (including SF3B1, SRSF2 and U2AF1) (OR 5.6, 95% C.I. 1.5-20.6), TET2 (OR 5.4, 95% C.I. 2.5-11.9) and DNMT3A (OR 2.7, 95% C.I. 1.6-4.6) were associated with significantly increased odds of developing AML relative to controls and are thus referred to as “high risk genes” hereafter. The odds ratio was elevated for JAK2 (OR 3.8, 95% C.I. 0.8-18.7), but did not reach statistical significance. However, the specific JAK2 mutation JAK2 p. Val617Phe was significantly associated with AML development (OR 6.1, 95% C.I. 1.2-61.1, P=0.03). Mutations in IDH1 and IDH2 were exclusively found in the known recurring hotspots in arginine-132 or arginine-140. Similarly, SRSF2 mutations were confined to the well-known proline-95 hotspot. TP53 mutations were primarily in the DNA binding, transactivation, and oligomerization domains. The distribution of mutations within functional protein domains, as well as the type of mutations found at each location, is shown in FIGS. 7A-7B, 8A-8B, 9, and 10A-10B, and 14A-14E, and 15-16.

### Example 4: Mutations are Associated with an Accelerated Time to AML Presentation

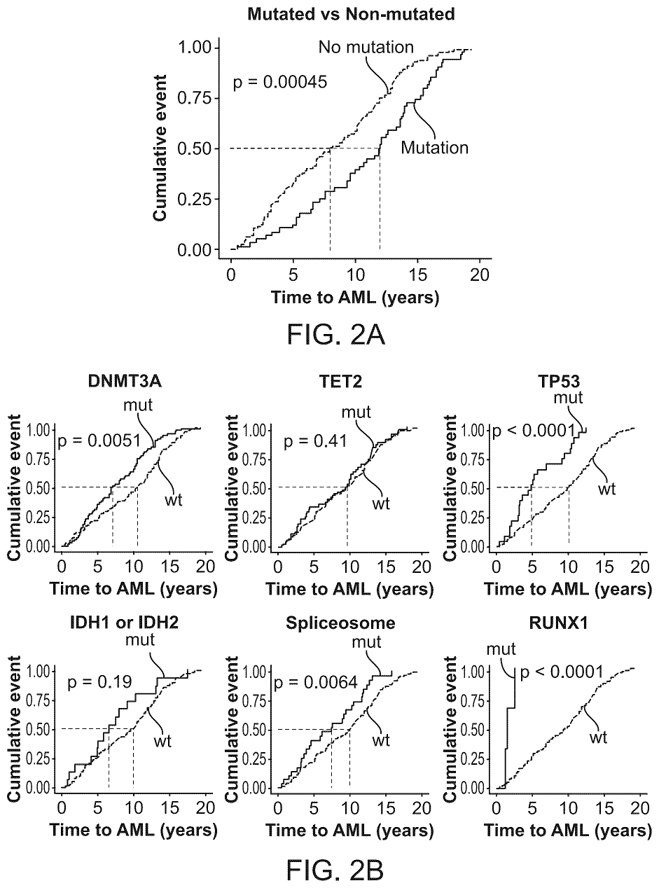

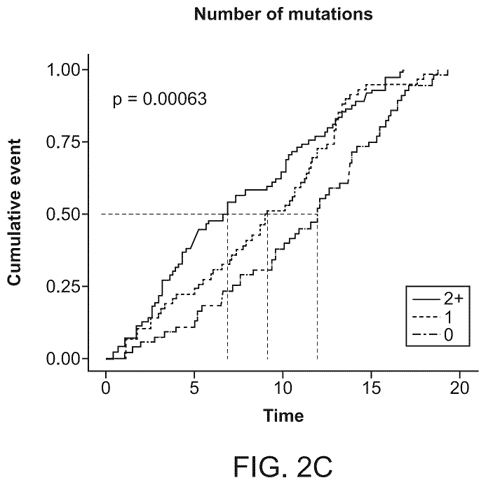

AML cases with mutations at baseline experienced significantly shorter latency of disease than cases without baseline mutations. The presence of any mutation shifted the median time to AML diagnosis from 11.9 years (no mutations) to a median of 8 years after baseline assessment (P<0.001, log rank test; FIG. 2A). Univariable analysis of mutated genes demonstrated that median time to AML varied according to mutated gene (FIG. 2B): DNMT3A (7.1 vs. 10.6 years), TP53 (4.9 vs. 10.2 years), spliceosome genes (7.4 vs. 10.0 years) and RUNX1 (1.5 vs. 9.6 years). The presence of RUNX1 mutations seemed to be associated with especially rapid development of AML within 2 years, but the number of participants in this subgroup was small (N=3). Clonal complexity also affected the latency of disease as patients with 1 mutation in the high risk genes developed AML in 9.1 years, but those with 2 or more mutations in the high risk genes developed AML in only 6.9 years (FIG. 2C).

Multivariable analyses produced similar findings. Table 1C shows a Forest plot indicating odds of developing AML within 5 years from baseline, depicted as odds ratios for the specific mutations. Mutations in TP53 and DNMT3A were significantly associated with rapid development of AML. IDH category includes IDH1 and IDH2. The spliceosome category includes SRSF2, SF3B1, and U2AF1.

TABLE 1C
Odds ratio: AML diagnosis < 5 years from baseline

| Gene | N | Odds Ratio (95% CI) | p value |
|---|---|---|---|
| TP53 | 23 | 3.63 (1.41, 9.32) | 0.006 |
| DNMT3A | 71 | 2.00 (1, 4.01) | 0.046 |
| Spliceosome | 27 | 1.61 (0.63, 4.15) | 0.306 |
| IDH | 15 | 1.44 (0.43, 4.83) | 0.539 |
| TET2 | 48 | 1.33 (0.6, 2.98) | 0.480 |
| JAK2 | 11 | 0.61 (0.13, 2.99) | 0.520 |
| Age | 189 | 0.98 (0.93, 1.04) | 0.483 |

Abbreviations: CI, confidence interval; N, number affected. P-values are shown for penalized likelihood multivariable logistic regression.

Mutations in TP53 were independently associated with AML occurring within 5 years of baseline (OR 3.6, 95% C.I. 1.4-9.1, P=0.006) as well as an elevated annual rate of AML (HR 2.8, 95% C.I. 1.7-4.7, P<0.001) when adjusted for confounding co-mutations and age. Similarly, DNMT3A mutations presented a smaller, but still significant odds of developing AML within 5 years (OR 2.9, 95% C.I. 1.0-4.1, P=0.04), as well as an elevated annual rate (HR 1.6, 95% C.I. 1.2-2.2, P=0.002). For TP53 and DNMT3A mutations, there was no appreciable difference in time to development of AML by mutation subtype, e.g., arginine-882 in DNMT3A. Finally, IDH mutations had an elevated annual rate of similar magnitude as DNMT3A (HR 1.6, 95% C.I. 0.9-2.8, P=0.09), but this did not achieve statistical significance. However, while 15/16 participants with IDH mutations were eventually diagnosed with AML, there was no elevated risk of AML earlier than 5 years after baseline assessment, suggesting the possibility that further downstream mutational events are required for AML development.

Complete Summary of Mutations

| Gene | Variant_Classification | Protein_Change |
|---|---|---|
| DNMT3A | In_Frame_Del | p.Leu731del |
| DNMT3A | Missense_Mutation | p.Gly543Cys |
| DNMT3A | Missense_Mutation | p.Phe752Ser |
| DNMT3A | Missense_Mutation | p.Gly543Cys |
| DNMT3A | Missense_Mutation | p.Phe752Ser |
| DNMT3A | Missense_Mutation | p.Arg635Gly |
| DNMT3A | Missense_Mutation | p.Arg882Cys |
| DNMT3A | Missense_Mutation | p.Arg635Gly |
| DNMT3A | Missense_Mutation | p.Arg882Cys |
| DNMT3A | Missense_Mutation | p.Trp306Cys |
| IDH2 | Missense_Mutation | p.Arg140Gln |
| DNMT3A | Frame_Shift_Ins | p.Gln110AlafsTer14 |
| TET2 | Nonsense_Mutation | p.Gln644Ter |
| JAK2 | Missense_Mutationn | p.Val617Phe |
| TET2 | Nonsense_Mutation | p.Gln644Ter |
| JAK2 | Missense_Mutationn | p.Val617Phe |
| ASXL1 | Frame_Shift_Del | p.Tyr700IlefsTer3 |
| JAK2 | Missense_Mutation | p.Val617Phe |
| JAK2 | Missense_Mutation | p.Val617Phe |
| NRAS | Missense_Mutation | p.Pro185Ser |
| DNMT3A | Missense_Mutation | p.Arg882Cys |
| DNMT3A | Missense_Mutation | p.Gly726Val |
| FLT1 | Missense_Mutation | p.Val395Ile |
| SF3B1 | Missense_Mutation | p.Arg625Leu |
| SRSF2 | Missense_Mutation | p.Pro95His |
| DNMT3A | Missense_Mutation | p.Phe731Leu |
| GNAS | In_Frame_Del | p.SerThrSerArgSerTerLeuArgGlyIleArgSerArgSerArgLeuArgGlySerProCysArgProSerArgSerArgLeuArgGlyGlyProTer416delextTer? |
| DNMT3A | Missense_Mutation | p.Arg882Cys |
| ASXL2 | Missense_Mutation | p.Ser685Leu |
| ASXL1 | Nonsense_Mutation | p.Gln692Ter |
| ASXL1 | Nonsense_Mutation | p.Gly679Ter |
| SRSF2 | Missense_Mutation | p.Pro95Leu |
| IDH2 | Missense_Mutation | p.Arg140Gln |
| TET2 | Nonsense_Mutation | p.Gln1510Ter |
| IDH2 | Missense_Mutation | p.Arg140Gln |
| TET2 | Nonsense_Mutation | p.Gln1510Ter |
| SRSF2 | Missense Mutation | p.Pro95Leu |
| DNMT3A | Missense_Mutation | p.Leu905Pro |
| DNMT3A | Missense_Mutation | p.Leu905Pro |
| FLT3 | Missense_Mutation | p.Leu198Phe |
| DNMT3A | Missense_Mutation | p.Arg736His |
| IDH2 | Missense_Mutation | p.Arg140Gln |
| DNMT3A | Missense_Mutation | p.Gly308Arg |
| DNMT3A | Splice_Site | NA |
| DNMT3A | Splice_Site | NA |
| DNMT3A | Missense_Mutation | p.Gly308Arg |
| DNMT3A | Missense_Mutation | p.Pro904Leu |
| CEBPA | Missense_Mutation | p.Asp279Glu |
| TET2 | Frame_Shift_Ins | p.Ser167PhefsTer4 |
| TP53 | Frame_Shift_Del | p.Leu93ArgfsTer30 |
| TP53 | Missense_Mutation | p.Arg337Cys |
| TP53 | Nonsense_Mutation | p.Gln167Ter |
| TP53 | Nonsense_Mutation | p.Gln167Ter |
| TP53 | Frame_Shift_Del | p.Leu93ArgfsTer30 |
| TP53 | Missense_Mutation | p.Arg337Cys |
| DNMT3A | Missense_Mutation | p.Arg882His |
| DNMT3A | Missense_Mutation | p.Arg882His |
| SRSF2 | Missense_Mutation | p.Pro95His |
| SRSF2 | Missense_Mutation | p.Pro95His |
| DNMT3A | Missense_Mutation | p.Ser337Leu |
| TET2 | Missense_Mutation | p.His1912Tyr |
| TET2 | Nonsense_Mutation | p.Gln180Ter |
| TET2 | Nonsense_Mutation | p.Gln180Ter |
| TET2 | Missense_Mutation | p.His1912Tyr |
| DNMT3A | Missense_Mutation | p.Ser337Leu |
| DNMT3A | Frame_Shift_Del | p.Lys766ArgfsTer13 |
| TET2 | Nonsense_Mutation | p.Gln1523Ter |
| SRSF2 | Missense_Mutation | p.Pro95His |
| TET2 | In_Frame_Del | p.Pro1356_Glu1357del |
| SF3B1 | Missense_Mutation | p.Lys790Glu |
| SF3B1 | Missense_Mutation | p.Gly742Asp |
| SRSF2 | Missense_Mutation | p.Pro95His |
| TET2 | Missense_Mutation | p.Glu1318Gly |
| DNMT3A | Nonsense_Mutation | p.Arg320Ter |
| TET2 | Missense_Mutation | p.Cys1263Arg |
| PHF6 | Nonsense_Mutation | p.Tyr301Ter |
| CBL | Frame_Shift_Ins | p.Val581CysfsTer4 |
| RUNX1 | Frame_Shift_Del | p.Met347IlefsTer251 |
| EZH2 | Missense_Mutation | p.Cys539Arg |
| TET2 | Missense_Mutation | p.Glu1874Gln |
| DNMT3A | Missense_Mutation | p.Pro777Leu |
| TET2 | Missense_Mutation | p.Asn140Ser |
| TET2 | Frame_Shift_Ins | p.Asn140ThrfsTer8 |
| TET2 | Nonsense_Mutation | p.Gln892Ter |
| TET2 | Frame_Shift_Ins | p.Ile1105TyrfsTer25 |
| TET2 | Frame_Shift_Del | p.Leu1780SerfsTer38 |
| TET2 | Frame_Shift_Del | p.Val291GlyfsTer2 |
| TET2 | Frame_Shift_Ins | p.Ile1105TyrfsTer25 |
| TET2 | Frame_Shift_Del | p.Leu1780SerfsTer38 |
| TET2 | Nonsense_Mutation | p.Gln892Ter |
| IDH2 | Missense_Mutation | p.Arg140Gln |
| IDH2 | Missense_Mutation | p.Arg140Gln |
| DNMT3A | Missense_Mutation | p.Tyr533Cys |
| DNMT3A | Missense_Mutation | p.Arg326Cys |
| DNMT3A | Missense_Mutation | p.Phe755Ser |
| IDH1 | Missense_Mutation | p.Arg132Cys |
| SRSF2 | Missense_Mutation | p.Pro95Leu |
| U2AF1 | Missense_Mutation | p.Arg156His |
| TET2 | Frame_Shift_Del | p.Asp302ValfsTer6 |
| TET2 | Frame_Shift_Del | p.Asp302ValfsTer6 |
| U2AF1 | Missense_Mutation | p.Arg156His |
| DNMT3A | Missense_Mutation | p.Arg882His |
| BRAF | Missense_Mutation | p.Lys601Asn |
| IDH2 | Missense_Mutation | p.Arg140Gln |
| SRSF2 | Missense_Mutation | p.Pro95Arg |
| TET1 | Missense_Mutation | p.Gln176Lys |
| TET1 | Missense_Mutation | p.Gln176Lys |
| TET2 | Nonsense_Mutation | p.Gln706Ter |
| DNMT3A | Missense_Mutation | p.Arg882His |
| IDH2 | Missense_Mutation | p.Arg140Gln |
| BRAF | Missense_Mutation | p.Lys601Asn |
| TP53 | Missense_Mutation | p.Leu145Pro |
| ASXL1 | Frame_Shift_Del | p.Ser964PhefsTer20 |
| TET2 | Frame_Shift_Del | p.Val291TrpfsTer2 |
| TET2 | Splice_Site | NA |
| NRAS | Missense_Mutation | p.Gly13Asp |
| TET2 | Frame_Shift_Del | p.Val291TrpfsTer2 |
| TET2 | Splice_Site | NA |
| DNMT3A | Missense_Mutation | p.Arg882Ser |
| DNMT3A | Missense_Mutation | p.Arg882Ser |
| TET2 | Splice_Site | NA |
| SRSF2 | Missense_Mutation | p.Pro95Thr |
| TET2 | Frame_Shift_Del | p.Glu320AsnfsTer27 |
| TET2 | Frame_Shift_Del | p.His786LeufsTer27 |
| ASXL1 | Nonsense_Mutation | p.Gln588Ter |
| TET2 | Frame_Shift_Del | p.Leu1515AlafsTer62 |
| ASXL1 | Nonsense_Mutation | p.Gln588Ter |
| TET2 | Frame_Shift_Del | p.His786LeufsTer27 |
| TET2 | Frame_Shift_Del | p.Leu1515AlafsTer62 |
| ASXL1 | Nonsense_Mutation | p.Gln588Ter |
| TET2 | Frame_Shift_Del | p.Leu1515AlafsTer62 |
| DNMT3A | Missense_Mutation | p.Arg882Cys |
| DNMT3A | Missense_Mutation | p.Val657Met |
| TET2 | Frame_Shift_Del | p.Asn1489MetfsTer82 |
| CUX1 | Splice_Site | NA |
| TET2 | Frame_Shift_Del | p.Arg1451GlyfsTer7 |
| TET2 | Frame_Shift_Del | p.Met695CysfsTer5 |
| TP53 | Nonsense_Mutation | p.Lys321Ter |
| SRSF2 | Missense_Mutation | p.Pro95His |
| TP53 | Nonsense_Mutation | p.Lys321Ter |
| TET2 | Frame_Shift_Del | p.Met695CysfsTer5 |
| WT1 | Missense_Mutation | p.Pro197Ser |
| TET2 | Frame_Shift_Del | p.Asn1489MetfsTer82 |
| TET2 | Frame_Shift_Del | p.Arg1451GlyfsTer7 |
| DNMT3A | Nonsense_Mutation | p.Trp313Ter |
| DNMT3A | Missense_Mutation | p.Arg326His |
| DNMT3A | Nonsense_Mutation | p.Trp313Ter |
| DNMT3A | Missense_Mutation | p.Arg326His |
| DNMT3A | Nonsense_Mutation | p.Tyr533Ter |
| TET2 | Missense_Mutation | p.Leu1151Pro |
| JAK2 | Missense_Mutation | p.Val617Phe |
| NOTCH1 | Frame_Shift_Del | p.Ser2530ArgfsTer75 |
| NRAS | Missense_Mutation | p.Gly12Asp |
| FLT3 | Missense_Mutation | p.Asp835His |
| TET2 | Frame_Shift_Del | p.Val647TrpfsTer53 |
| TET2 | Nonsense_Mutation | p.Tyr1337Ter |
| TET2 | Missense_Mutation | p.Cys1378Tyr |
| SF3B1 | Missense_Mutation | p.Lys700Glu |
| TET2 | Frame_Shift_Del | p.Ala727HisfsTer23 |
| DNMT3A | Frame_Shift_Del | p.Phe336SerfsTer9 |
| TET2 | Frame_Shift_Del | p.Glu1320ArgfsTer43 |
| PAX5 | Missense_Mutation | p.Pro32Ser |
| PAX5 | Missense Mutation | p.Pro32Ser |
| DNMT3A | Frame_Shift_Del | p.Phe336SerfsTer9 |
| TET2 | Frame_Shift_Del | p.Ala727HisfsTer23 |
| TET2 | Frame_Shift_Del | p.Glu1320ArgfsTer43 |
| DNMT3A | Missense_Mutation | p.Arg882Pro |
| DNMT3A | Missense_Mutation | p.Arg882Pro |
| TP53 | Nonsense_Mutation | p.Gln100Ter |
| DNMT3A | Missense_Mutation | p.Val328Phe |
| TP53 | Nonsense_Mutation | p.Gln100Ter |
| DNMT3A | Missense_Mutation | p.Val328Phe |
| SF3B1 | Missense Mutation | p.Lys700Glu |
| TP53 | Missense_Mutation | p.Arg248Trp |
| TP53 | Missense Mutation | p.Arg273His |
| DNMT3A | Missense_Mutation | p.Phe755Ser |
| DNMT3A | Missense_Mutation | p.Phe755Ser |
| TP53 | Missense_Mutation | p.Arg273His |
| DNMT3A | Splice_Site | NA |
| TET2 | Frame_Shift_Ins | p.Trp954LeufsTer18 |
| IDH2 | Missense_Mutation | p.Arg140His |
| DNMT3A | Nonsense_Mutation | p.Arg598Ter |
| DNMT3A | Missense_Mutation | p.Ser770Leu |
| DNMT3A | Missense_Mutation | p.Ser770Leu |
| DNMT3A | Missense_Mutation | p.Thr862Ile |
| DNMT3A | Nonsense_Mutation | p.Arg598Ter |
| DNMT3A | Missense_Mutation | p.His873Pro |
| ASXL1 | Nonsense_Mutation | p.Gln780Ter |
| IDH2 | Missense_Mutation | p.Arg140Trp |
| U2AF1 | Missense_Mutation | p.Gln157Arg |
| ASXL1 | Nonsense_Mutation | p.Arg693Ter |
| IDH2 | Missense_Mutation | p.Arg140Trp |
| U2AF1 | Missense_Mutation | p.Gln157Arg |
| GNAS | Missense_Mutation | p.Gly9Ser |
| TET2 | Frame_Shift_Ins | p.Tyr1148LeufsTer9 |
| DNMT3A | Missense_Mutation | p.Cys557Gly |
| DNMT3A | Missense_Mutation | p.Cys557Gly |
| TET2 | Frame_Shift_Ins | p.Tyr1148LeufsTer9 |
| TP53 | Missense_Mutation | p.Ala161Thr |
| TP53 | Missense_Mutation | p.Arg175Gly |
| U2AF1 | In_Frame_Ins | p.Tyr158dup |
| U2AF1 | In_Frame_Ins | p.Tyr158dup |
| TP53 | Missense_Mutation | p.Ala161Thr |
| PHF6 | Missense_Mutation | p.Leu324Pro |
| IKZF1 | Missense_Mutation | p.Asn159Ser |
| RUNX1 | Frame_Shift_Del | p.Pro57ArgfsTer15 |
| BCOR | Nonsense_Mutation | p.Gln416Ter |
| DNMT3A | Missense_Mutation | p.Arg882His |
| DNMT3A | Missense_Mutation | p.Arg688His |
| DNMT3A | Missense_Mutation | p.Arg882His |
| SRSF2 | Missense_Mutation | p.Pro95His |
| TP53 | Missense_Mutation | p.Tyr163His |
| TET2 | Missense_Mutation | p.His1881Tyr |
| TET2 | Missense Mutation | p.Val1900Gly |
| CBL | Missense_Mutation | p.Phe434Ser |
| TET2 | Frame_Shift_Del | p.Leu1511TrpfsTer60 |
| CBL | Missense_Mutation | p.Phe434Ser |
| TET2 | Missense_Mutation | p.Val1900Gly |
| SRSF2 | Missense_Mutation | p.Pro95His |
| TET2 | Frame_Shift_Del | p.Leu1511TrpfsTer60 |
| TET2 | Nonsense_Mutation | p.Glu537Ter |
| TET2 | Splice_Site | NA |
| TET2 | Splice_Site | NA |
| TET2 | Splice_Site | NA |
| TET2 | Splice_Site | NA |
| JAK2 | Missense_Mutation | p.Val617Phe |
| DNMT3A | Frame_Shift_Del | p.Gly413SerfsTer238 |
| TET2 | Nonsense_Mutation | p.Gln764Ter |
| TET2 | Frame_Shift_Ins | p.Asp1427ValfsTer22 |
| PHF6 | Missense_Mutation | p.Phe263Val |
| TET2 | Frame_Shift_Ins | p.Leu920SerfsTer2 |
| TET2 | Splice_Site | NA |
| DNMT3A | Missense_Mutation | p.Arg882His |
| IDH2 | Missense_Mutation | p.Arg140Gln |
| KRAS | Missense_Mutation | p.Gly12Arg |
| SRSF2 | Missense_Mutation | p.Pro95Arg |
| IDH2 | Missense_Mutation | p.Arg140Gln |
| TP53 | Missense_Mutation | p.Tyr236His |
| DNMT3A | Frame_Shift_Del | p.Val759TrpfsTer20 |
| DNMT3A | Frame_Shift_Del | p.Phe303SerfsTer13 |
| DNMT3A | Frame_Shift_Del | p.Phe303SerfsTer13 |
| DNMT3A | Frame_Shift_Del | p.Val759TrpfsTer20 |
| DNMT3A | Nonsense_Mutation | p.Arg771Ter |
| BCOR | Frame_Shift_Del | p.Thr1557ProfsTer11 |
| DNMT3A | Nonsense_Mutation | p.Arg771Ter |
| DNMT3A | Missense_Mutation | p.Arg882His |
| TET2 | Nonsense_Mutation | p.Gln740Ter |
| DNMT3A | Missense_Mutation | p.Glu774Asp |
| DNMT3A | Missense_Mutation | p.Glu774Asp |
| CREBBP | In_Frame_Ins | p.Asn494_Gln495insProAspAlaAlaAlaAla |
| DNMT3A | Frame_Shift_Del | p.Pro416LeufsTer235 |
| DNMT3A | Frame_Shift_Del | p.Pro416LeufsTer235 |
| CREBBP | In_Frame_Ins | p.Asn494_Gln495insProAspAlaAlaAlaAla |
| TP53 | Missense_Mutation | p.Cys275Tyr |
| SF3B1 | Missense_Mutation | p.Lys700Glu |
| SF3B1 | Missense_Mutation | p.Lys700Glu |
| TET2 | Missense_Mutation | p.His1912Tyr |
| TET2 | Missense_Mutation | p.His1912Tyr |
| TET2 | Frame_Shift_Ins | p.Pro1594GlnfsTer37 |
| TET2 | Missense_Mutation | p.Ile1873Asn |
| TP53 | Missense Mutation | p.Arg248Trp |
| JAK2 | Missense_Mutation | p.Val617Phe |
| JAK2 | Missense_Mutation | p.Val617Phe |
| TP53 | Missense Mutation | p.Arg248Trp |
| DNMT3A | Frame_Shift_Del | p.Cys710AlafsTer69 |
| TET2 | Missense_Mutation | p.Gly1361Asp |
| TET2 | Nonsense_Mutation | p.Leu500Ter |
| DNMT3A | Frame_Shift_Del | p.Cys710AlafsTer69 |
| TET2 | Nonsense_Mutation | p.Leu500Ter |
| TET2 | Missense_Mutation | p.Gly1361Asp |
| SRSF2 | Missense_Mutation | p.Pro95Leu |
| IDH2 | Missense_Mutation | p.Arg140Gln |
| TET2 | Nonsense_Mutation | p.Gly773Ter |
| KDM6A | Splice_Site | NA |
| TET2 | Nonsense_Mutation | p.Gly773Ter |
| SRSF2 | Missense_Mutation | p.Pro95Leu |
| IDH2 | Missense_Mutation | p.Arg140Gln |
| KDM6A | Splice_Site | NA |
| DNMT3A | Frame_Shift_Del | p.Phe751SerfsTer28 |
| DNMT3A | Frame_Shift_Del | p.Phe751SerfsTer28 |
| DNMT3A | Missense_Mutation | p.Arg882His |
| GATA1 | Missense_Mutation | p.Pro14Leu |
| DNMT3A | Missense_Mutation | p.Gly293Arg |
| TET2 | Nonsense_Mutation | p.Gln321Ter |
| TET2 | Nonsense_Mutation | p.Gln745Ter |
| SRSF2 | Missense_Mutation | p.Pro95Leu |
| GNAS | Missense_Mutation | p.Arg844His |
| SETBP1 | Missense_Mutation | p.Gly870Ser |
| IDH2 | Missense_Mutation | p.Arg140Gln |
| SF3B1 | Missense_Mutation | p.Ala263Val |
| CBL | Nonsense_Mutation | p.Arg206Ter |
| SF3B1 | Missense_Mutation | p.Lys666Asn |
| TET2 | Frame_Shift_Del | p.Asp1858SerfsTer10 |
| DNMT3A | Missense_Mutation | p.Arg736His |
| SRSF2 | Missense_Mutation | p.Pro95Leu |
| IDH2 | Missense_Mutation | p.Arg140Gln |
| TP53 | Missense_Mutation | p.Arg248Trp |
| SRSF2 | Missense_Mutation | p.Pro95Leu |
| IDH2 | Missense_Mutation | p.Arg140Gln |
| CBL | Nonsense_Mutation | p.Trp303Ter |
| IDH2 | Missense_Mutation | p.Arg140Gln |
| DNMT3A | Missense_Mutation | p.Gly728Asp |
| DNMT3A | Nonsense_Mutation | p.Trp330Ter |
| DNMT3A | Missense_Mutation | p.Pro743His |
| U2AF1 | Missense_Mutation | p.Gln157Arg |
| DNMT3A | Missense_Mutation | p.Pro743His |
| CUX1 | Nonsense_Mutation | p.IleArgArgGluLeuPheIleGluGlu1310TrpSerProTyrHisTerLysLysLys |
| TET2 | Missense_Mutation | p.Ala1158Val |
| TP53 | Missense_Mutation | p.Ile195Ser |
| DNMT3A | Nonsense_Mutation | p.Trp330Ter |
| TET2 | Nonsense_Mutation | p.Ser1494Ter |
| TET2 | Missense_Mutation | p.Cys1875Gly |
| TET2 | Nonsense_Mutation | p.Leu719Ter |
| TP53 | Frame_Shift_Del | p.Pro128LeufsTer42 |
| TET2 | Missense_Mutation | p.Ala1876Val |
| TP53 | Missense_Mutation | p.Tyr220Cys |
| DNMT3A | Missense_Mutation | p.Arg882His |
| DNMT3A | Missense_Mutation | p.Arg882His |
| TET2 | Missense_Mutation | p.Cys1875Gly |
| TP53 | Frame_Shift_Del | p.Pro128LeufsTer42 |
| TET2 | Missense_Mutation | p.Ala1876Val |
| JAK2 | Missense_Mutation | p.Val617Phe |
| JAK2 | Missense_Mutation | p.Val617Phe |
| TET2 | Nonsense_Mutation | p.Gln705Ter |
| TET2 | Missense_Mutation | p.Ser1870Leu |
| ETV6 | Nonsense_Mutation | p.Trp380Ter |
| DNMT3A | Missense_Mutation | p.Arg882His |
| U2AF1 | Missense_Mutation | p.Gln157Arg |
| ETV6 | Nonsense_Mutation | p.Tyr344Ter |
| RUNX1 | Missense_Mutation | p.Pro425Leu |
| TET2 | Splice_Site | NA |
| SRSF2 | Missense_Mutation | p.Pro95His |
| DNMT3A | Nonsense_Mutation | p.Trp860Ter |
| DNMT3A | Missense_Mutation | p.Leu737Arg |
| TET2 | Missense_Mutation | p.His1386Asp |
| U2AF1 | Missense_Mutation | p.Gln157Arg |
| TET2 | Missense_Mutation | p.Ala1158Val |
| TET2 | Missense_Mutation | p.Gln1414His |
| TP53 | Missense_Mutation | p.Ile195Ser |
| TP53 | Missense_Mutation | p.Val272Met |
| TET2 | Missense_Mutation | p.Ala1158Val |
| U2AF1 | Missense_Mutation | p.Gln157Arg |
| TP53 | Missense_Mutation | p.Val272Met |
| TET2 | Missense_Mutation | p.Gln1414His |
| TP53 | Missense_Mutation | p.Ile195Ser |
| JAK2 | Missense_Mutation | p.Val617Phe |
| TP53 | Missense_Mutation | p.Cys242Tyr |
| SF3B1 | Missense_Mutation | p.Ala744Val |
| TET2 | Frame_Shift_Ins | p.Asn442LysfsTer19 |
| TP53 | Missense_Mutation | p.Cys242Tyr |
| TET2 | Frame_Shift_Ins | p.Asn442LysfsTer19 |
| SF3B1 | Missense_Mutation | p.Ala744Val |
| JAK2 | Missense_Mutation | p.Val617Phe |
| TP53 | Missense_Mutation | p.Val272Met |
| TP53 | Missense_Mutation | p.Val272Met |
| TET2 | Missense_Mutation | p.Lys664Glu |
| ASXL1 | Nonsense_Mutation | p.Arg404Ter |
| ASXL1 | Nonsense_Mutation | p.Arg404Ter |
| TET2 | Missense_Mutation | p.Lys664Glu |
| DNMT3A | Splice_Site | NA |
| TP53 | Missense_Mutation | p.Tyr220Cys |
| DNMT3A | Missense_Mutation | p.Arg882His |
| TET2 | Nonsense_Mutation | p.Gln706Ter |
| TET2 | Nonsense_Mutation | p.Arg1452Ter |
| KRAS | Missense_Mutation | p.Gly12Asp |
| KRAS | Missense_Mutation | p.Gly12Asp |
| IDH2 | Missense_Mutation | p.Arg140Gln |
| NPM1 | Frame_Shift_Ins | p.Trp288CysfsTer12 |
| IDH2 | Missense_Mutation | p.Arg140Gln |
| JAK1 | Missense_Mutation | p.Gly1097Ala |
| DNMT3A | Missense_Mutation | p.Arg882His |
| SF3B1 | Missense_Mutation | p.Lys700Glu |
| SF3B1 | Missense_Mutation | p.Lys700Glu |
| DNMT3A | Frame_Shift_Del | p.Ala254HisfsTer62 |
| DNMT3A | Frame_Shift_Del | p.Ala254HisfsTer62 |
| CALR | Frame_Shift_Del | p.Lys368ThrfsTer51 |
| CALR | Frame_Shift_Del | p.Lys368ThrfsTer51 |
| DNMT3A | Frame_Shift_Del | p.Val830Ter |
| CARD1 | Missense_Mutation | p.Ala817Thr |
| DNMT3A | Frame_Shift_Del | p.Val830Ter |
| TP53 | Frame_Shift_Del | p.Asn29ThrfsTer15 |
| TP53 | Frame_Shift_Del | p.Arg333ValfsTer12 |
| TP53 | Frame_Shift_Del | p.Asn29ThrfsTer15 |
| DNMT3A | Splice_Site | NA |
| DNMT3A | Missense_Mutation | p.Gly706Glu |
| SRSF2 | Missense_Mutation | p.Pro95His |
| DNMT3A | Missense Mutation | p.Gly706Glu |
| SRSF2 | Missense_Mutation | p.Pro95His |
| TET2 | Missense_Mutation | p.Ser1898Pro |
| DNMT3A | Missense_Mutation | p.Arg882Cys |
| DNMT3A | Frame_Shift_Del | p.Gln485ArgfsTer166 |
| DNMT3A | Missense_Mutation | p.Arg882Cys |
| DNMT3A | Frame_Shift_Del | p.Gln485ArgfsTer166 |
| TET2 | Missense_Mutation | p.Ser1898Pro |
| DNMT3A | Missense_Mutation | p.Arg882His |
| DNMT3A | Missense_Mutation | p.Pro804Leu |
| DNMT3A | Missense_Mutation | p.Ala368Asp |
| DNMT3A | Missense_Mutation | p.Tyr528Asn |
| U2AF1 | Missense_Mutation | p.Gln157Pro |
| IDH1 | Missense_Mutation | p.Arg132His |
| JAK2 | Missense_Mutation | p.Val617Phe |
| TET2 | Missense_Mutation | p.Cys1378Arg |
| JAK2 | Missense_Mutation | p.Val617Phe |
| IDH1 | Missense_Mutation | p.Arg132His |
| ASXL1 | Nonsense_Mutation | p.Arg693Ter |
| TP53 | Missense_Mutation | p.Met246Ile |
| DNMT3A | Missense_Mutation | p.Phe732Ser |
| DNMT3A | Missense_Mutation | p.Arg736Cys |
| DNMT3A | Nonsense_Mutation | p.Tyr536Ter |
| DNMT3A | Nonsense_Mutation | p.Tyr536Ter |
| DNMT3A | Missense_Mutation | p.Val296Met |
| DNMT3A | Nonsense_Mutation | p.Trp795Ter |
| DNMT3A | Missense_Mutation | p.Val296Met |
| DNMT3A | Missense_Mutation | p.Arg882His |
| DNMT3A | Missense_Mutation | p.Trp698Gly |
| DNMT3A | Missense_Mutation | p.Asp531Asn |
| TET2 | Nonsense_Mutation | p.Gln734Ter |
| CARD1 | Missense_Mutation | p.Arg866Gln |
| ASXL1 | Frame_Shift_Del | p.Leu739ProfsTer5 |
| TET2 | Missense_Mutation | p.His1904Leu |
| CBL | Missense_Mutation | p.Val896Phe |
| TET2 | Nonsense_Mutation | p.Gln734Ter |
| CARD1 | Missense_Mutation | p.Arg866Gln |
| CBL | Missense_Mutation | p.Val896Phe |
| TET2 | Missense_Mutation | p.His1904Leu |
| ASXL1 | Frame_Shift_Del | p.Leu739ProfsTer5 |
| TET2 | Frame_Shift_Ins | p.Thr556AsnfsTer11 |
| DNMT3A | Frame_Shift_Ins | p.Thr503AsnfsTer43 |
| DNMT3A | Frame_Shift_Ins | p.Thr503AsnfsTer43 |
| TET2 | Frame_Shift_Ins | p.Thr556AsnfsTer11 |
| FLT1 | Missense Mutation | p.Ile536Val |
| DNMT3A | Missense_Mutation | p.Pro904Leu |
| IDH1 | Missense_Mutation | p.Arg132Gly |
| CARD1 | Missense_Mutation | p.Arg866Gln |
| DNMT3A | Missense_Mutation | p.Pro904Leu |
| IDH1 | Missense_Mutation | p.Arg132Gly |
| JAK2 | Missense_Mutation | p.Val617Phe |
| IDH2 | Missense_Mutation | p.Arg140Gln |
| SRSF2 | Missense_Mutation | p.Pro95His |
| JAK2 | Missense_Mutation | p.Val617Phe |
| TET2 | Missense_Mutation | p.Cys1263Tyr |
| DNMT3A | Missense_Mutation | p.Pro904Leu |
| TET2 | Missense_Mutation | p.Cys1263Tyr |
| DNMT3A | Missense_Mutation | p.Pro904Leu |
| TET2 | Frame_Shift_Del | p.Pro1644HisfsTer51 |
| TET2 | Frame_Shift_Ins | p.Gly641ArgfsTer40 |
| TET2 | Frame_Shift_Del | p.Pro1644HisfsTer51 |
| PHF6 | Missense Mutation | p.Ala238Thr |
| TET2 | Missense_Mutation | p.Glu1879Val |
| TET2 | Missense_Mutation | p.His1904Arg |
| TET2 | Missense_Mutation | p.Ala1512Val |
| SF3B1 | Missense_Mutation | p.His662Asp |
| SF3B1 | Missense_Mutation | p.His662Asp |
| CBLB | Splice_Site | NA |
| EZH2 | Nonsense_Mutation | p.Trp15Ter |
| EZH2 | Nonsense_Mutation | p.Trp15Ter |
| TET2 | Missense_Mutation | p.His1904Gln |
| TP53 | Missense_Mutation | p.Pro278His |
| TP53 | Missense_Mutation | p.Arg248Trp |
| TET2 | Frame_Shift_Ins | p.Met1333TyrfsTer6 |
| TP53 | Missense_Mutation | p.Tyr220Cys |
| TP53 | Missense_Mutation | p.Tyr220Cys |
| TET2 | Frame_Shift_Ins | p.Met1333TyrfsTer6 |
| IDH2 | Missense_Mutation | p.Arg140Gln |
| SRSF2 | Missense_Mutation | p.Pro95Leu |
| CUX1 | Missense_Mutation | p.Arg583His |
| NOTCH1 | Missense Mutation | p.Arg912Trp |
| CBL | Missense_Mutation | p.Arg575Gly |
| SRSF2 | Missense_Mutation | p.Pro95Leu |
| IDH2 | Missense_Mutation | p.Arg140Gln |
| RAD21 | Frame_Shift_Ins | p.Glu331ArgfsTer4 |
| DNMT3A | Splice_Site | NA |
| CEBPA | Frame_Shift_Ins | p.His24ArgfsTer137 |
| CEBPA | Missense_Mutation | p.Arg289Pro |
| ATRX | Splice_Site | NA |
| GATA2 | Missense_Mutation | p.Tyr322Asn |
| PHF6 | Frame_Shift_Del | p.Arg335MetfsTer15 |
| TP53 | Missense_Mutation | p.Asn239Ser |
| TP53 | Missense_Mutation | p.Val143Met |
| DNMT3A | Missense_Mutation | p.Pro904Gln |
| TET2 | Frame_Shift_Ins | p.Ile1160TyrfsTer2 |
| TET2 | Frame_Shift_Ins | p.Ile1160TyrfsTer2 |
| DNMT3A | Missense_Mutation | p.Pro904Gln |
| TET2 | Missense_Mutation | p.Arg1359Ser |
| TET2 | Frame_Shift_Del | p.Asn258MetfsTer35 |
| TET2 | Frame_Shift_Ins | p.Lys1299Ter |
| GNAS | In_Frame_Del | p.SerThrSerArgSerTerLeuArgGlyIleArgSerArgSerArgLeuArgGlySerProCysArgProSerArgSerArgr416delextTer? |
| JAK2 | Missense_Mutation | p.Val617Phe |
| DNMT3A | Missense Mutation | p.Tyr735Cys |
| SF3B1 | Missense_Mutation | p.Arg625Cys |
| TET2 | Frame_Shift_Ins | p.Ala1174LysfsTer53 |
| DNMT3A | Missense_Mutation | p.Phe848Ser |
| JAK2 | Missense_Mutation | p.Val617Phe |
| DNMT3A | Missense_Mutation | p.Tyr735Cys |
| SF3B1 | Missense_Mutation | p.Arg625Cys |
| DNMT3A | Missense_Mutation | p.Phe848Ser |
| FLT1 | Missense_Mutation | p.Asp1052Asn |
| CALR | Missense_Mutation | p.Asp244Gly |
| STAG1 | Missense_Mutation | p.Ala116Thr |
| STAT6 | Frame_Shift_Ins | p.Gln281ProfsTer92 |
| FLT1 | Missense Mutation | p.Arg312His |
| TET1 | Missense_Mutation | p.Leu1107Phe |
| FLT3 | Frame_Shift_Del | p.Pro986AlafsTer27 |
| NOTCH1 | Missense_Mutation | p.Pro668Ser |
| KRAS | Missense_Mutation | p.Gly13Asp |
| TET2 | Frame_Shift_Del | p.Asn442ThrfsTer5 |
| DNMT3A | Missense_Mutation | p.Arg882His |
| KDM6A | Frame_Shift_Del | p.Gly19ValfsTer23 |
| DNMT3A | Missense_Mutation | p.Arg882His |
| TET2 | Frame_Shift_Del | p.Asn442ThrfsTer5 |
| TP53 | Missense_Mutation | p.Arg175His |
| TET2 | Nonsense_Mutation | p.Arg1516Ter |
| TP53 | Missense_Mutation | p.Arg175His |
| TET2 | Nonsense_Mutation | p.Arg1516Ter |
| CUX1 | Nonsense_Mutation | p.Trp12Ter |
| U2AF1 | Missense_Mutation | p.Gln157Pro |
| CUX1 | Frame_Shift_Ins | p.Leu967SerfsTer37 |
| SETBP1 | Missense_Mutation | p.Asp868Tyr |
| CBL | Missense_Mutation | p.Arg420Gln |
| CUX1 | Frame_Shift_Ins | p.Leu967SerfsTer37 |
| ASXL1 | Nonsense_Mutation | p.Arg1415Ter |
| CBL | Missense_Mutation | p.Arg420Gln |
| SRSF2 | Missense_Mutation | p.Pro95Arg |
| U2AF1 | Missense_Mutation | p.Gln157Pro |
| TET2 | Missense_Mutation | p.Gly1275Arg |
| FAM46 | Missense_Mutation | p.Tyr371Cys |
| TET2 | Missense_Mutation | p.Ile1873Thr |
| TET2 | Missense_Mutation | p.Gly1275Arg |
| FAM46 | Missense_Mutation | p.Tyr371Cys |
| TET2 | Missense_Mutation | p.Ile1873Thr |
| IKZF1 | Missense_Mutation | p.Asn159Ser |
| RUNX1 | Frame_Shift_Ins | p.Leu112CysfsTer13 |
| RUNX1 | Missense_Mutation | p.Ala142Val |
| SF3B1 | Missense_Mutation | p.His662Asp |
| DNMT3A | Frame_Shift_Ins | p.Phe794LeufsTer4 |
| SF3B1 | Missense_Mutation | p.His662Asp |
| DNMT3A | Frame_Shift_Ins | p.Phe794LeufsTer4 |
| DNMT3A | Missense_Mutation | p.Lys906Glu |
| DNMT3A | Nonsense_Mutation | p.Arg771Ter |
| DNMT3A | Missense_Mutation | p.Lys906Glu |
| DNMT3A | Nonsense_Mutation | p.Arg771Ter |
| DNMT3A | Frame_Shift_Ins | p.Ile670HisfsTer43 |
| TP53 | Missense_Mutation | p.Thr155Asn |
| DNMT3A | Frame_Shift_Ins | p.Ile670HisfsTer43 |
| TET2 | Nonsense_Mutation | p.Trp1847Ter |
| CBL | Missense_Mutation | p.Arg420Pro |
| CBL | Missense_Mutation | p.Arg420Pro |
| EZH2 | Missense_Mutation | p.Phe670Leu |
| DNMT3A | Missense_Mutation | p.Pro904Leu |
| DNMT3A | Splice_Site | NA |
| DNMT3A | Missense_Mutation | p.Ile705Thr |
| TP53 | Missense_Mutation | p.Tyr234Cys |
| JAK2 | Missense Mutation | p.Val617Phe |
| TP53 | Frame_Shift_Del | p.Phe109SerfsTer14 |
| DNMT3A | Missense_Mutation | p.Arg635Trp |
| DNMT3A | Missense_Mutation | p.Ile705Thr |
| DNMT3A | Missense Mutation | p.Ile705Thr |
| DNMT3A | Missense_Mutation | p.Arg635Trp |
| JAK2 | Missense_Mutation | p.Val617Phe |
| DNMT3A | Splice_Site | NA |
| DNMT3A | Missense_Mutation | p.Val895Met |
| SF3B1 | Missense_Mutation | p.Lys700Glu |
| TP53 | Splice_Site | NA |
| TP53 | Missense_Mutation | p.Cys275Phe |
| DNMT3A | Nonsense_Mutation | p.Arg771Ter |
| GNAS | Missense_Mutation | p.Gly9Ser |
| TET2 | Frame_Shift_Ins | p.Thr229AsnfsTer25 |
| CBLC | Missense_Mutation | p.Val333Met |
| DNMT3A | Nonsense_Mutation | p.Trp409Ter |
| DNMT3A | Nonsense_Mutation | p.Trp409Ter |
| ASXL1 | Frame_Shift_Ins | p.Lys1045GlnfsTer5 |
| ASXL1 | Frame_Shift_Ins | p.Lys1045GlnfsTer5 |
| ATRX | Missense_Mutation | p.Gly124Asp |
| JAK2 | Missense_Mutation | p.Gly48Glu |
| GNAS | Missense_Mutation | p.Arg1016Cys |
| PHF6 | In_Frame_Del | p.Thr255del |
| DNMT3A | Frame_Shift_Ins | p.Leu605AspfsTer7 |
| DNMT3A | Frame_Shift_Ins | p.Leu605AspfsTer7 |
| TET2 | Nonsense_Mutation | p.Tyr1294Ter |
| DNMT3A | Missense_Mutation | p.Arg882Cys |
| KDM6A | Missense_Mutation | p.Val139Ile |
| KDM6A | Missense_Mutation | p.Val139Ile |
| TET2 | Nonsense_Mutation | p.Arg1712Ter |
| DNMT3A | Nonsense_Mutation | p.Glu725Ter |
| DNMT3A | Frame_Shift_Del | p.Ser393ValfsTer14 |
| ASXL1 | Nonsense_Mutation | p.Gly679Ter |
| IDH2 | Missense_Mutation | p.Arg140Gln |
| KDM6A | Missense_Mutation | p.Ala1033Asp |
| SRSF2 | Missense_Mutation | p.Pro95Leu |
| DNMT3A | Frame_Shift_Del | p.Gly796ValfsTer6 |
| DNMT3A | Frame_Shift_Del | p.Gly796ValfsTer6 |
| TET2 | Frame_Shift_Del | p.Asn1489MetfsTer82 |
| TET2 | Frame_Shift_Del | p.Asn1489MetfsTer82 |
| FLT1 | Missense_Mutation | p.Val395Ile |
| FLT1 | Missense_Mutation | p.Val395Ile |
| DNMT3A | Missense_Mutation | p.Cys537Arg |
| DNMT3A | Missense_Mutation | p.Arg882His |
| SF3B1 | Missense_Mutation | p.Ala263Val |
| ASXL1 | Frame_Shift_Del | p.Asp879LeufsTer6 |
| SF3B1 | Missense_Mutation | p.Lys666Asn |
| DNMT3A | Missense_Mutation | p.Phe414Val |
| NRAS | Missense_Mutation | p.Pro185Ser |
| DNMT3A | Frame_Shift_Ins | p.Gln816AlafsTer42 |
| DNMT3A | Frame_Shift_Ins | p.Gln816AlafsTer42 |
| DNMT3A | Missense_Mutation | p.Ala368Thr |
| DNMT3A | Missense_Mutation | p.Ala368Thr |
| NOTCH1 | Missense_Mutation | p.Arg1350Leu |
| DNMT3A | Nonsense_Mutation | p.Trp314Ter |
| DNMT3A | Splice_Site | NA |
| HRAS | Missense_Mutation | p.Leu133Arg |
| HRAS | Missense_Mutation | p.Leu133Arg |
| TET2 | Splice_Site | NA |
| DNMT3A | Missense_Mutation | p.Gly550Arg |
| DNMT3A | Missense_Mutation | p.Gly550Arg |
| TET2 | Splice_Site | NA |
| GNAS | In_Frame_Del | p.Thr407_Ala436del |
| DNMT3A | Nonsense_Mutation | p.Glu561Ter |
| FLT3 | Missense_Mutation | p.Leu198Phe |
| NOTCH1 | Missense_Mutation | p.Pro2525Leu |
| DNMT3A | Missense_Mutation | p.Glu733Gly |
| TET2 | Missense_Mutation | p.Tyr1902Cys |
| DNMT3A | Missense_Mutation | p.Tyr908Cys |
| DNMT3A | Missense_Mutation | p.Tyr908Cys |
| TET2 | Missense_Mutation | p.Tyr1902Cys |
| DNMT3A | Frame_Shift_Del | p.Pro849LeufsTer4 |
| DNMT3A | Frame_Shift_Del | p.Pro849LeufsTer4 |
| DNMT3A | Missense_Mutation | p.Ala910Pro |
| DNMT3A | Splice_Site | NA |
| DNMT3A | Missense_Mutation | p.Trp860Arg |
| TET2 | Frame_Shift_Ins | p.Leu200ThrfsTer2 |
| TET2 | Frame_Shift_Ins | p.Leu200ThrfsTer2 |
| DNMT3A | Missense_Mutation | p.Arg326Cys |
| DNMT3A | Missense_Mutation | p.Arg326Cys |
| DNMT3A | Frame_Shift_Del | p.Cys666TrpfsTer39 |
| DNMT3A | Frame_Shift_Del | p.Cys666TrpfsTer39 |
| BCOR | Missense_Mutation | p.Arg540Trp |
| FLT1 | Missense_Mutation | p.Val395Ile |
| TET2 | Missense_Mutation | p.Gly1282Cys |
| DNMT3A | Frame_Shift_Del | p.Arg458GlyfsTer193 |
| TET2 | Missense_Mutation | p.Asp1376Gly |
| ASXL1 | Missense_Mutation | p.Arg92His |
| DNMT3A | Missense_Mutation | p.Arg882His |
| DNMT3A | Missense_Mutation | p.Trp305Gly |
| ASXL1 | Nonsense_Mutation | p.Gln592Ter |
| DNMT3A | Frame_Shift_Del | p.Asn797ThrfsTer5 |
| DNMT3A | Frame_Shift_Del | p.Asn797ThrfsTer5 |
| DNMT3A | Splice_Site | NA |
| FLT1 | Nonsense_Mutation | p.Lys371Ter |
| FLT1 | Nonsense_Mutation | p.Lys371Ter |
| DNMT3A | Splice_Site | NA |
| NOTCH1 | Missense_Mutation | p.Arg912Trp |
| CBL | Missense_Mutation | p.Arg575Gly |
| DNMT3A | Missense_Mutation | p.Arg882Cys |
| DNMT3A | Missense_Mutation | p.Arg882Cys |
| DNMT3A | Missense_Mutation | p.Arg688His |
| DNMT3A | Missense_Mutation | p.Arg688His |
| DNMT3A | Missense_Mutation | p.Ala368Val |
| DNMT3A | Missense_Mutation | p.Ala368Val |
| DNMT3A | Missense_Mutation | p.Arg326Cys |
| DNMT3A | Missense_Mutation | p.Arg882Cys |
| DNMT3A | In_Frame_Del | p.Leu731del |
| DNMT3A | Missense_Mutation | p.Arg326Cys |
| DNMT3A | In_Frame_Del | p.Leu731del |
| DNMT3A | Missense_Mutation | p.Arg882Cys |
| JAK2 | Missense_Mutation | p.Glu814Gly |
| JAK2 | Missense_Mutation | p.Val617Phe |
| KDM6A | Nonsense_Mutation | p.Glu657Ter |
| JAK2 | Missense_Mutation | p.Val617Phe |
| JAK2 | Missense_Mutation | p.Glu814Gly |
| KIT | Missense_Mutation | p.Ser821Phe |
| TET2 | Frame_Shift_Del | p.Val1056LeufsTer10 |
| DNMT3A | Frame_Shift_Del | p.Val502AspfsTer43 |
| ASXL1 | Missense_Mutation | p.Arg92His |
| CSF3R | Missense Mutation | p.Gly5Ala |
| CUX1 | Missense_Mutation | p.Arg609His |
| CREBBP | Nonsense_Mutation | p.Gln203Ter |
| CSF3R | Missense_Mutation | p.Gly5Ala |
| CUX1 | Missense_Mutation | p.Arg609His |
| CREBBP | Nonsense_Mutation | p.Gln203Ter |
| SETBP1 | Missense_Mutation | p.Ile402Val |
| TET2 | Splice_Site | NA |
| TET2 | Splice_Site | NA |
| DNMT3A | Missense_Mutation | p.Ile780Thr |
| DNMT3A | Missense_Mutation | p.Ile780Thr |
| DNMT3A | Frame_Shift_Del | p.Met852IlefsTer29 |
| DNMT3A | Missense_Mutation | p.Pro849Ser |
| DNMT3A | Nonsense_Mutation | p.Trp314Ter |
| DNMT3A | Nonsense_Mutation | p.Trp601Ter |
| DNMT3A | Nonsense_Mutation | p.Trp601Ter |
| DNMT3A | Missense_Mutation | p.Met761Val |
| DNMT3A | Missense_Mutation | p.Met761Val |
| CARD1 | Missense_Mutation | p.Ala817Thr |
| ASXL1 | Missense_Mutation | p.Arg489Gln |
| TET2 | Frame_Shift_Ins | p.Cys973LeufsTer3 |
| CBLC | Missense_Mutation | p.Val333Met |
| DNMT3A | Missense_Mutation | p.Tyr533Cys |
| DNMT3A | Missense_Mutation | p.Tyr533Cys |
| DNMT3A | Missense_Mutation | p.Asn797Lys |
| DNMT3A | Missense_Mutation | p.Asn797Lys |
| GNAS | In_Frame_Del | p.Thr407_Ala436del |
| DNMT3A | Splice_Site | NA |
| DNMT3A | Missense_Mutation | p.Arg736His |
| TET2 | Splice_Site | NA |
| CBL | Missense_Mutation | p.Ile383Met |
| JAK1 | Missense_Mutation | p.Lys696Ile |
| DNMT3A | Splice_Site | NA |
| DNMT3A | Missense_Mutation | p.Arg899Cys |
| DNMT3A | Missense_Mutation | p.Tyr735Cys |
| GNAS | Missense_Mutation | p.Arg1016Cys |
| JAK2 | Missense_Mutation | p.Gly48Glu |
| DNMT3A | Missense_Mutation | p.Arg882Cys |
| SF3B1 | Missense_Mutation | p.Lys666Arg |
| DNMT3A | Splice_Site | NA |
| DNMT3A | Splice_Site | NA |
| GNAS | In_Frame_Del | p.Thr407_Ala436del |
| DNMT3A | Missense_Mutation | p.Arg301Trp |
| ASXL1 | In_Frame_Del | p.Gln207del |
| DNMT3A | Missense_Mutation | p.Arg301Trp |
| JAK1 | Missense_Mutation | p.Gly1097Val |
| DNMT3A | Missense_Mutation | p.Arg749Gly |
| TET2 | Frame_Shift_Del | p.Gln1547LeufsTer19 |
| TET2 | Frame_Shift_Del | p.Gln1547LeufsTer19 |
| TET2 | Frame_Shift_Del | p.Leu1276TrpfsTer87 |
| ASXL1 | Missense_Mutation | p.Pro808Ser |

Fs = frameshift,
Ter = STOP codon

### Example 5: Mutations Occurring at any Variant Allele Fraction (VAF) in High-Risk Genes are Associated with Increased Odds of AML

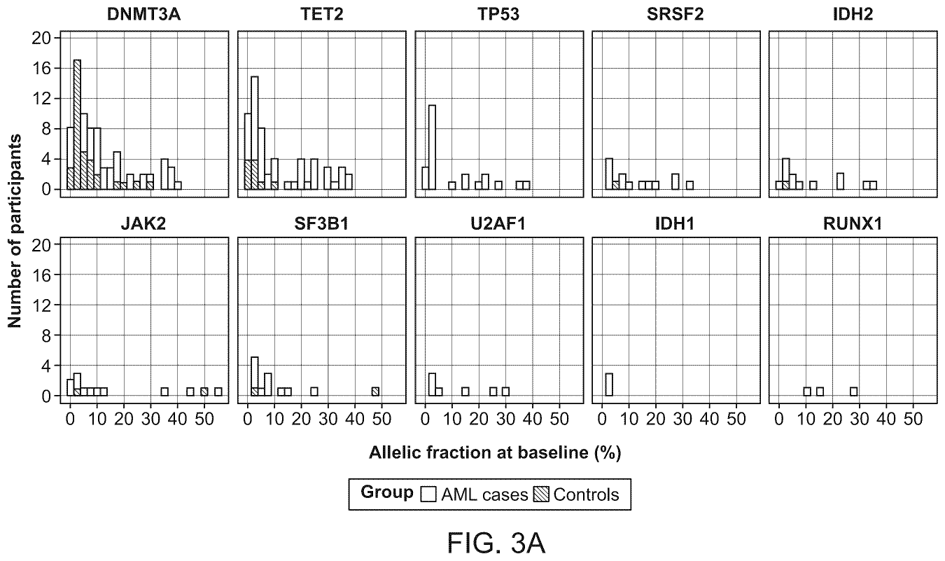

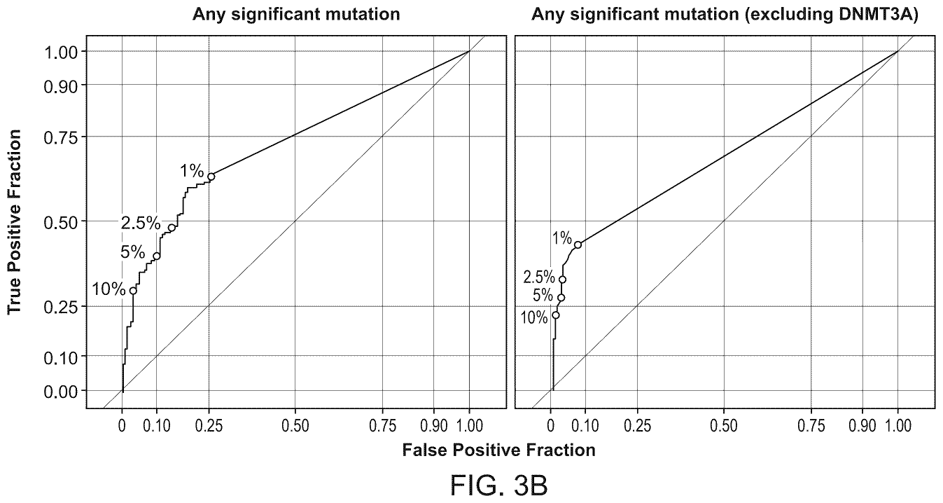

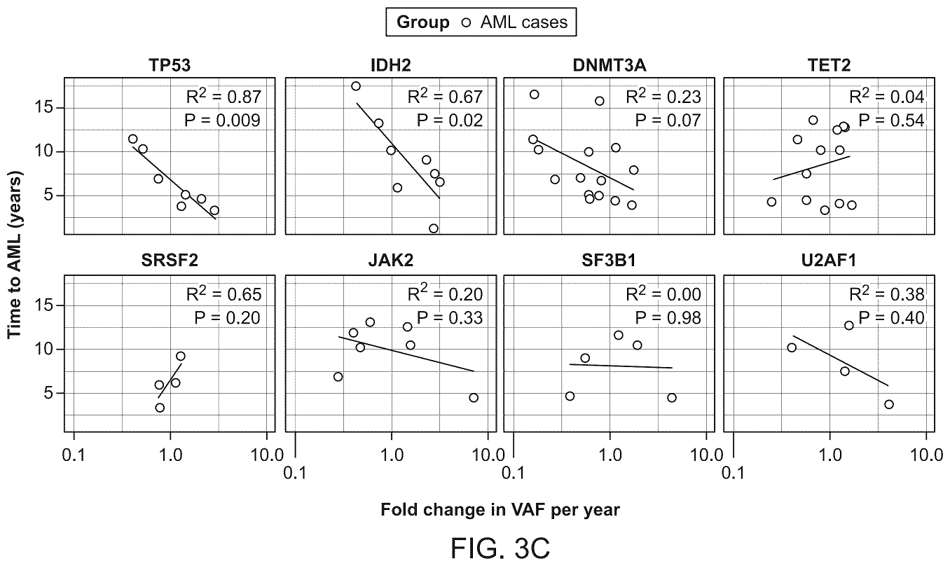

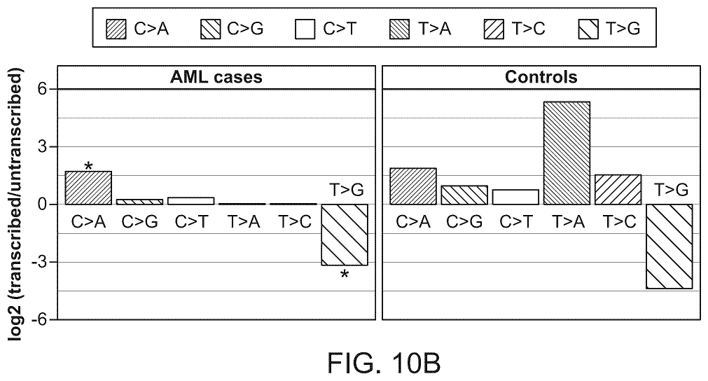

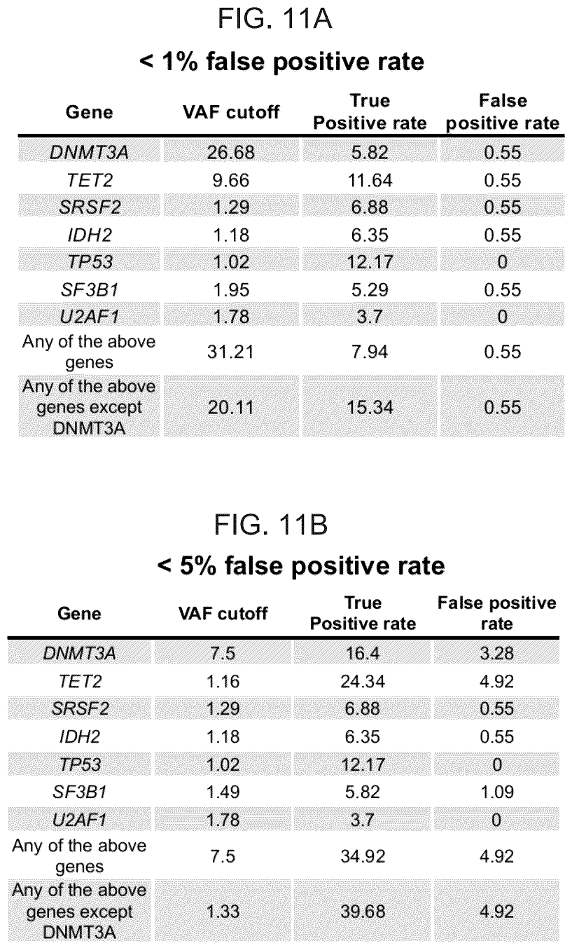

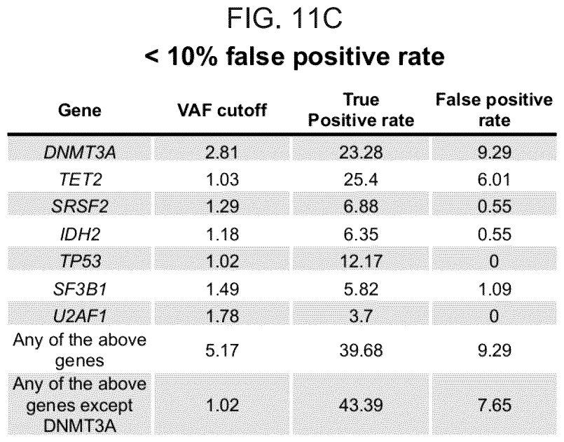

Of participants harboring any mutation evaluated at VAF >10% at baseline, 83% eventually developed AML (OR 6.5, 95% C.I. 3.4-13.0, P<0.001). When only mutations in genes associated with development of AML shown in FIG. 3A were considered, mutations present at VAF >10% at baseline led to AML in 90% of cases (OR 11.6, 95% C.I. 5.1-31.1, P<0.001). As demonstrated in the histograms, mutations in DNMT3A and TET2 at lower VAFs were notably more distributed among cases and controls. In contrast, mutations present at higher VAFs in DNMT3A and TET2 were almost exclusively seen in AML cases, suggesting that mutations in DNMT3A and TET2 are less specific for AML cases at lower VAFs. Thus, when excluding DNMT3A and TET2, 92% of patients harboring mutations at any VAF above the 1% cutoff (range 1.0%-54.9% VAF) in the remaining genes eventually developed AML (OR 16.5, 95% C.I. 6.4-54.0, P<0.001). Next, the specificity of mutations in genes significantly associated with AML cases (Table 1B) were further examined by determining their frequency in the controls. The true positive and false positive rate of mutations at varying VAF cutoffs was visualized using receiver operating characteristic (ROC) analysis (FIG. 3B). At a VAF cutoff of 1.3%, the presence of a mutation in any gene (excluding DNMT3A) resulted in a 4.9% rate of false positive detection (controls misclassified as cases). Individually, mutations in TP53, SRSF2, U2AF1, SF3B1, and IDH2 produced a ˜1% rate of false positive detection at VAF cutoffs ranging from 1-2%. Tabulated results are available in FIGS. 11A-11C.

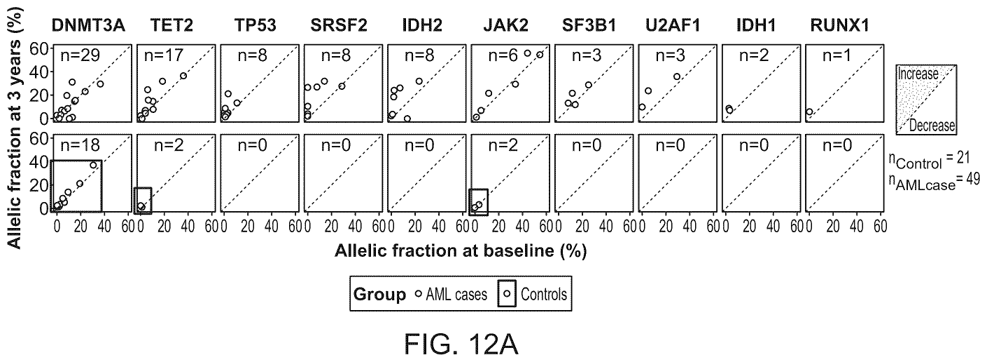

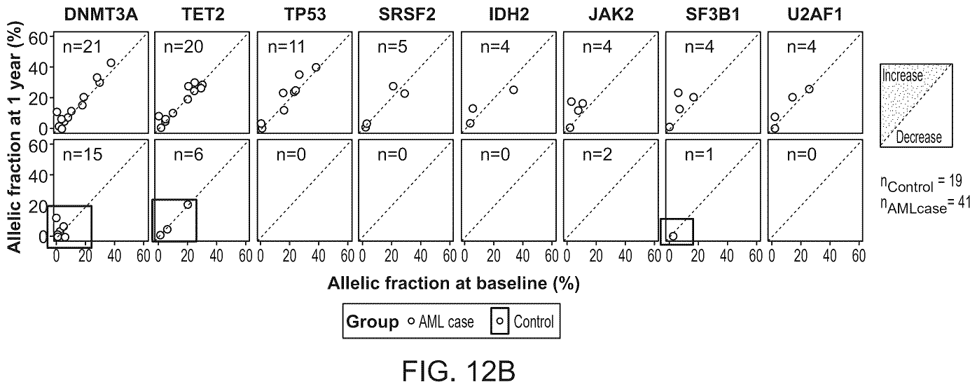

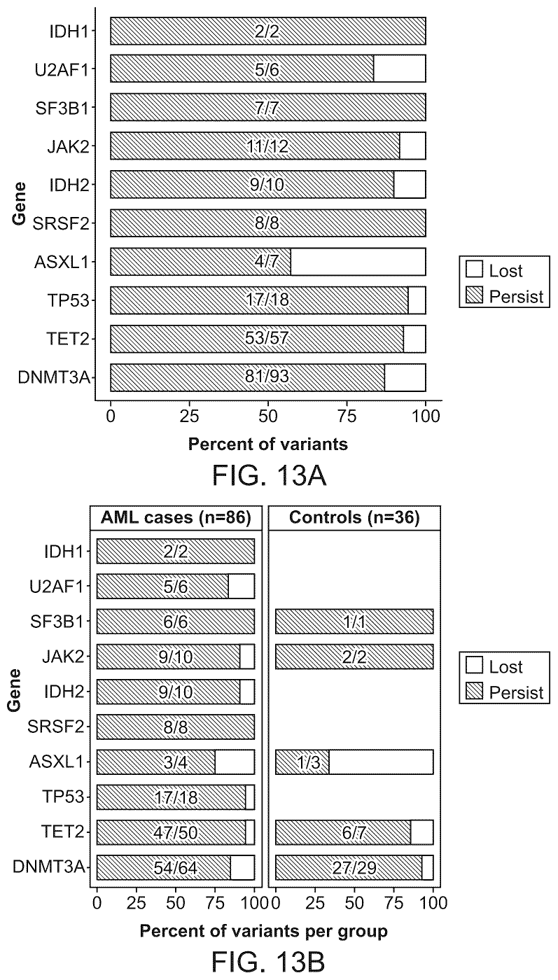

For the subset of participants for whom serial samples were available, changes in VAF were assessed from baseline to year 1 or year 3. We evaluated whether the time to AML development was influenced by the fold increase in VAF in participants who had demonstrated a statistically significant rise in VAF upon serial testing (FIG. 3C). The rate of increase in VAF for IDH2 mutations (R2=0.87, slope-3.3, 95% C.I.-21.5-3.7, P=0.02) and TP53 mutations (R2=0.67, slope-3.0, 95% C.I.-14.0-5.5, P=0.009) was significantly associated with a shorter time to AML in a linear regression analysis. While this relationship was similar for mutations in DNMT3A, it did not achieve statistical significance. There were fewer changes from baseline in VAF across all genes in year 1, compared to year 3 (FIGS. 12A-12B). Finally, for participants with serial samples available, 89% of mutations detected in the study persisted from baseline to years 1 or 3, irrespective of whether the VAF significantly changed (FIGS. 13A-13B).

### Example 6: Association Between Non-AML Cancer History and Mutations

The mutational patterns of the 22 cases and 21 controls that had a history of malignancy at baseline, including breast, lung, bladder, endometrial, ovarian, colon cancer and lymphoma were examined. Treatment history for the cancer was not known. Forty-five percent of AML cases (N=10/22) and 33% of controls (N=7/21) with a prior history of cancer at baseline were found to have mutations (not significant). While the absolute number of cases and controls with prior cancer history was few, 3/10 cases with prior history of cancer harbored IDH2 mutations at baseline evaluation and all 3 of these cases had prior breast cancer. None of the cases or controls with a prior history of cancer had TP53 gene mutations.

### Example 7: Progression to AML from Baseline Mutations

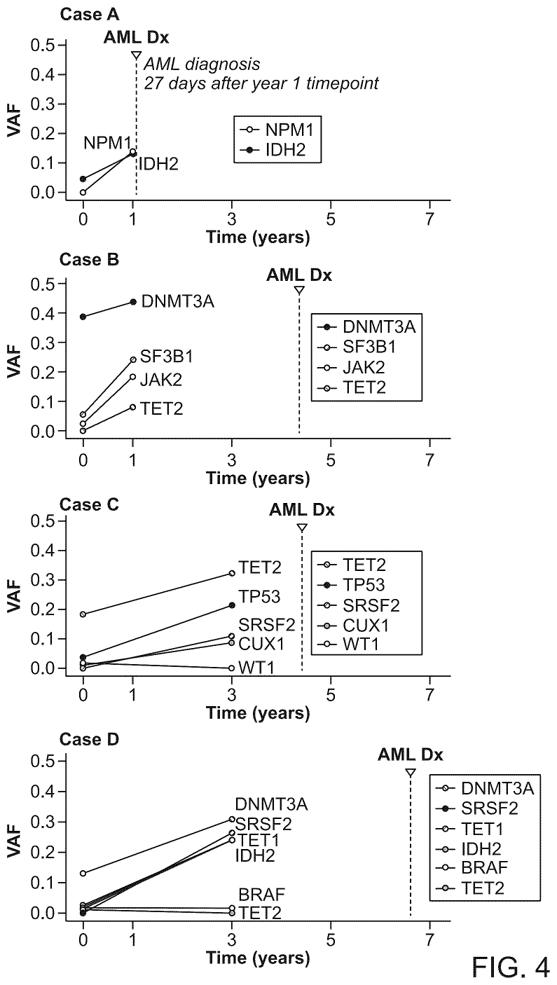

Despite having selected for participants who eventually developed AML, NPM1 and FLT3 mutations, which are among the two most frequently recurring driver mutations in AML (Mardis, E. R. et al., N Engl J Med 361, 1058-1066 (2009); Palmisano, M. et al., Haematologica 92, 1268-1269 (2007)), were absent. This finding was consistent with other reports of their absence in clonal hematopoiesis, and suggests that they may play a cooperative role in AML pathogenesis. A single case was identified where follow up at year 1 preceded AML diagnosis by <30 days (FIG. 4, Case A) and mutations at baseline and year 1 (<30 days prior to AML diagnosis) were compared. The depth of sequencing coverage at NPM1 insertion sites was similar at both time points (˜450×). The participant had an IDH2 mutation (8% VAF) at baseline and after 1 year acquired a new NPM1 type A mutation (14% VAF), along with an increased VAF of the IDH2 mutation (13%). AML was diagnosed less than 30 days later. The rapid development of AML after the acquisition of an NPM1 mutation suggests cooperation with the pre-existing IDH2 clone. Other patterns of clonal evolution and expansion are demonstrated in representative cases B, C, and D (FIG. 4). Mutations in genes typically associated with clonal hematopoiesis, such as DNMT3A and TET2, were shown to generally have stable or minimally increased VAFs in follow-up. Progression to AML was often preceded by the acquisition of new mutations, or by the expansion of mutations in other genes, such as RUNX1 or TP53 (FIG. 4).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. (canceled)

2. (canceled)

3. (canceled)

4. (canceled)

5. (canceled)

6. (canceled)

7. A method for enhancing the efficacy of predicting the onset of AML symptoms or AML in a subject prior to the onset of AML symptoms comprising detecting the presence of one or more mutations in two or more of TP53, IDH1, IDH2, SRSF2, SF3B1, U2AF1, TET2, JAK2, and DNMT3A in a biological sample obtained from the subject, wherein the subject has not been diagnosed as having AML.

8. (canceled)

9. (canceled)

10. The method of claim 7, wherein the one or more mutations in two or more of TP53, IDH1, IDH2, SRSF2, SF3B1, U2AF1, TET2, JAK2, and DNMT3A are detected using PCR, Northern blots, Southern blots, microarray, dot or slot blots, in situ hybridization, electrophoresis, chromatography, mass spectroscopy, sedimentation, next-generation sequencing, Sanger sequencing, whole exome sequencing, targeted exome sequencing, error-corrected sequencing, augmented exome sequencing, whole genome sequencing, mRNA-seq or whole transcriptome RNA-seq.

11. The method of claim 10, wherein PCR comprises Real-time quantitative PCR (RQ-PCR), digital PCR, or reverse transcriptase PCR (RT-PCR).

12. The method of claim 7, further comprising detecting 2-hydroxyglutarate levels in the biological sample.

13. The method of claim 7, wherein the biological sample is a blood sample, a plasma sample, or a serum sample.

14. The method of claim 7, wherein the biological sample comprises one or more of genomic DNA, RNA, cDNA, cell-free DNA (cfDNA), cell-free RNA (cfRNA), and an exosome-associated nucleic acid.

15. The method of claim 7, wherein the mutation in SRSF2, DNMT3A, TET2, IDH2, IDH1, TP53, SF3B1, U2AF1, or JAK2 is a frameshift mutation, a missense mutation, a nonsense mutation, a splice site mutation, a duplication, an insertion mutation, and a deletion mutation.

16. The method of claim 7, further comprising detecting the presence of a mutation in one or more genes selected from the group consisting of ASXL1, ASXL2, BRAF, CALR, CARD11, CBL, CBLB, CBLC, CEBPA, CREBBP, CSF3R, CUX1, ETV6, EZH2, FLT1, FLT3, GATA1, GATA2, GNAS, HRAS, IKZF1, JAK1, KDM6A, KIT, KRAS, NOTCH1, NPM1, NRAS, PAX5, PHF6, RAD21, RUNX1, SETBP1, SF3B1,STAG1, STAT6, and TET1.

17. The method of claim 7, wherein the mutation in DNMT3A is selected from the group consisting of p.Leu731del, p.Gly543Cys, p.Phe752Ser, p.Arg635Gly, p.Arg882Cys, p.Trp306Cys, p.Gln110AlafsTer14, p.Gly726Val, p.Phe 731Lcu, p.Leu905Pro, p.Arg736His, p.Gly308Arg, p.Pro904Lcu, p.Arg882His, p.Ser337Leu, p.Lys766ArgfsTer13, p.Arg320Ter, p.Pro777Leu, p.Tyr533Cys, p.Arg326Cys, p.Phc755Ser, p.Arg882Ser, p.Val657Met, p.Trp313Ter, p.Arg326His, p.Tyr533Ter, p.Phe336SerfsTer9, p.Arg882Pro, p. Val328Phe, p.Arg598Ter, p.Ser770Leu, p.Thr862Ile, p.His873Pro, p.Cys557Gly, p.Arg688His, p.Gly413SerfsTer238, p. Val759TrpfsTer20, p.Phe303SerfsTer13, p.Arg771Ter, p.Glu774Asp, p.Pro416LeufsTer235, p.Cys710AlafsTer69, p.Phe 751 SerfsTer28, p.Gly293Arg, p.Gly728Asp, p.Trp330Ter, p.Pro743His, p. Trp860Ter, p.Leu737Arg, p.Ala254HisfsTer62, p. Val830Ter, p.Gly706Glu, p.Gln485ArgfsTer166, p.Pro804Lcu, p.Ala368Asp, p.Tyr528Asn, p.Phe732Ser, p.Arg736Cys, p.Tyr536Ter, p. Val296Met, p.Trp795Ter, p.Trp698Gly, p.Asp531 Asn, p.Thr503AsnfsTer43, p.Pro904Gln, p.Tyr735Cys, p.Phe848Ser, p.Phe794LcufsTer4, p.Lys906Glu, p.Ile670HisfsTer43, p.Ile705Thr, p.Arg635Trp, p. Val895Met, p.Trp409Ter, p.Leu605AspfsTer7, p.Glu725Ter, p.Ser393ValfsTer14, p.Gly796ValfsTer6, p.Cys537Arg, p.Phe414Val. p.Gln816AlafsTer42, p.Ala368Thr, p.Trp314Ter, p.Gly550Arg, p.Glu561Ter, p.Glu733Gly, p.Tyr908Cys, p.Pro849LeufsTer4, p.Ala910Pro, p.Trp860Arg, p.Cys666TrpfsTer39, p.Arg458GlyfsTer193, p.Trp305Gly, p.Asn797ThrfsTer5, p.Ala368Val, p. Val502AspfsTer43, p.Ile 780Thr, p.Met852IlefsTer29, p.Pro849Ser, p.Trp601Ter, p. Met761 Val, p.Asn797Lys, p.Arg899Cys, p.Arg301Trp, and p.Arg749Gly, or
wherein the mutation in IDH1 is p.Arg132Cys or p.Arg132Gly, or
wherein the mutation in IDH2 is p.Arg140Gln, p.Arg140His, or p.Arg 140Trp, or
wherein the mutation in JAK2 is p. Val617Phe, p.Gly48Glu, or p.Glu814Gly, or
wherein the mutation in SF3B1 is selected from the group consisting of p.Arg625Leu, p.Lys790Glu, p.Gly742Asp, p.Lys700Glu, p.Ala263 Val, p.Lys666Asn, p.Ala744Val, p.His662Asp, and p.Arg625Cys, or
wherein the mutation in SRSF2 is selected from the group consisting of p.Pro95His, p.Pro95Leu, p.Pro95Arg, and p.Pro95Thr, or
wherein the mutation in TET2 is selected from the group consisting of p.Gln644Ter, p.Gln 1510Ter, p.Ser167PhefsTer4, p.His 1912Tyr, p.Gln 180Ter, p.Gln 1523Ter, p.Pro1356_Glu1357del, p.Glu1318Gly, p.Cys1263Arg. p.Glu1874Gln, p.Asn140Ser, p.Asn 140ThrfsTer8, p.Gln892Ter, p.Ile 1 105TyrfsTer25, p. Leu 1 780SerfsTer38, p. Val291GlyfsTer2, p.Asp302ValfsTer6, p.Gln706Ter, p. Val291TrpfsTer2, p.Glu320AsnfsTer27, p.His786LeufsTer27, p.Leu1515AlafsTer62, p. Asn 1489MetfsTer82 p.Arg 1451GlyfsTer7, p.Met695CysfsTer5, p.Leu 1151Pro, p. Val647TrpfsTer53, p. Tyr 1337Ter, p.Cys 1378Tyr, p.Ala 727HisfsTer23, p. Glu 1320ArgfsTer43, p. Trp954LeufsTer18, p. Tyr1 148LeufsTer9, p. His 1881Tyr, p. Val 1900Gly, p. Leu 1511 TrpfsTer60, p.Glu537Ter, p.Gln764Ter, p.Asp 1427ValfsTer22, p.Leu920SerfsTer2, p.Gln740Ter, p.Pro1594GInfsTer37, p.Ile 1873Asn, p.Gly 1361Asp, p.Leu500Ter, p.Gly773Ter, p.Gln321Ter, p.Gln745Ter, p.Asp 1858SerfsTer 10, p.Ala 1 158Val, p.Ser1494Ter, p.Cys1875Gly, p.Leu719Ter, p.Ala 1876Val, p.Gln705Ter, p.Ser1870Leu, p.His 1386Asp, p.Gln 1414His, p.Asn442LysfsTer19, p.Lys664Glu, p.Arg 1452Ter, p.Ser1898Pro, p.Cys 1378Arg, p.Gln734Ter, p.His 1904Leu, p.Thr556AsnfsTer11, p.Cys1263Tyr, p.Pro1644HisfsTer51, p.Gly641 ArgfsTer40, p.Glu 1879Val, p.His 1904Arg, p.Ala 1512Val, p. His 1904Gln, p.Met 1333TyrfsTer6, p.Ile 1160TyrfsTer2, p.Arg1359Ser, p.Asn258MetfsTer35, p.Lys1299Ter, p.Alal 174LysfsTer53, p.Asn442ThrfsTer5, p.Arg1516Ter, p.Gly 1275Arg, p.Ile 1873Thr, p.Trp 1847Ter, p.Thr229AsnfsTer25, p.Tyr1294Ter, p.Arg1712Ter, p.Tyr1902Cys, p.Leu200ThrfsTer2, p.Gly 1282Cys, p.Asp1376Gly, p. Val1056LeufsTer10, p.Cys973LeufsTer3, p.Gln 1547LeufsTer19, and p. Leu 1276TrpfsTer87, or
wherein the mutation in TP53 is selected from the group consisting of p.Leu93ArgfsTer30, p.Arg337Cys, p.Gln167Ter, p.Leu145Pro, p.Lys321Ter, p.Gln100Ter, p.Arg248Trp, p.Arg273His, p.Ala161Thr, p.Arg175Gly, p. Tyr163His, p.Tyr236His, p.Cys275Tyr, p. Ile 195Ser, p.Pro 128LcufsTer42, p.Tyr220Cys, p. Val272Met, p.Cys242Tyr, p.Asn29ThrfsTer15, p.Arg333ValfsTer12, p.Mct246lle, p.Pro278His, p.Asn239Ser, p. Val143Met, p.Arg175His, p.Thr 155Asn, p. Tyr234Cys, p.Phe 109SerfsTer14, and p.Cys275Phe, or
wherein the mutation in U2AF1 is selected from the group consisting of p. Arg 156His, p.Gln 157Arg, p.Tyr158dup, and p.Gln 157Pro.

18. (canceled)

19. (canceled)

20. (canceled)

21. (canceled)

23. (canceled)

24. (canceled)

25. (canceled)

26. The method of claim 7, wherein the AML symptoms are selected from the group consisting of fever, fatigue, irregular heartbeat, dizziness, bone pain, frequent nosebleeds, bleeding and swollen gums, bruising on skin, loss of appetite, excessive sweating, shortness of breath, unexplained weight loss, headaches, diarrhea, menorrhagia, slurred speech, confusion, abdominal swelling, pale skin, seizures, vomiting, loss of balance, facial numbness, and blurred vision.

27. The method of claim 7, wherein the AML has a subtype selected from the group consisting of MO, M1, M2, M3, M4, M5, M6, M7 and M4Eo.

28. (canceled)

29. (canceled)

30. (canceled)

31. (canceled)

32. (canceled)

33. (canceled)

34. (canceled)

35. (canceled)

36. (canceled)

37. The method of claim 7, wherein the method comprises detecting the presence of one or more mutations in two or more of IDH1, IDH2, SRSF2, SF3B1, and U2AF1 in a biological sample obtained from the subject.
